(19) United States
(12) Patent Application Publication
(10) Pub. No.: US 2017/0226550 A1
(43) Pub. Date: Aug. 10, 2017

(54) INHIBITORY RNA FOR ENHANCED PROTEIN PRODUCTION IN RECOMBINANT MAMMALIAN CELLS

(71) Applicants: Jianxin Ye, East Brunswick, NJ (US); Krista Alvin, Basking Ridge, NJ (US); Siyan Zhang, Belle Mead, NJ (US)

(72) Inventors: Jianxin Ye, East Brunswick, NJ (US); Krista Alvin, Basking Ridge, NJ (US); Siyan Zhang, Belle Mead, NJ (US)

(73) Assignee: Merck Sharp & Dohme Corp., Rahway, NJ (US)

(21) Appl. No.: 15/501,503

(22) PCT Filed: Aug. 4, 2015
(86) PCT No.: PCT/US15/43523

§ 371 Date: Feb. 3, 2017

Related U.S. Application Data
(60) Provisional application No. 62/080,677, filed on Nov. 17, 2014.
(60) Provisional application No. 62/034,484, filed on Aug. 7, 2014.

(51) Int. Cl. C12P 21/00; C12N 15/113

(52) U.S. Cl. CPC C12P 21/00; C12N 15/113; C12N 2310/14; C12N 2310/531

(57) ABSTRACT

The invention provides methods and compositions for improving production of exogenous polypeptides in large scale culture of mammalian host cells. The methods and compositions utilize RNA interference to inhibit expression of one or more specific host cell proteins. The present invention is based on the identification of specific mammalian genes and sequences therein that are useful as RNAi targets to enhance the production of exogenous proteins in mammalian cell culture.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: One 118 KB ASCII (Text) file named “23806_SeqLIsting.Txt” created on Jul. 23, 2015.

### FIELD OF THE INVENTION

The present invention relates to the expression of exogenous polypeptides in mammalian host cells, and in particular to increasing the production of a desired exogenous polypeptide by inhibiting the host cell's expression of certain endogenous proteins.

### BACKGROUND OF THE INVENTION

Many biotherapeutic polypeptides, such as monoclonal antibodies and vaccines, are produced by culturing mammalian host cells transfected with an expression vector that drives constitutive and high level expression of the desired biotherapeutic polypeptide (see, e.g., Wurm, F. M., Nature Biotech. 22:1393-1398 (2004)). Various approaches have been explored to increase the productivity of recombinant mammalian cells and thereby lower production costs, including optimizing the activity of the expression vector, screening transfected host cells to select the highest producer and optimizing the cell culture medium and culture conditions. Increased productivity has also been accomplished by using RNA interference (RNAi) to selectivity inhibit expression of one or more endogenous host cell proteins that negatively influence production of the biotherapeutic polypeptide (see, e.g., WO2011/005786 and U.S. Pat. No. 8,273,722).

One challenge in utilizing RNAi to improve productivity of recombinant proteins in mammalian cells is identifying appropriate host cell genetic sequences to target. Protein production in mammalian cells is a very complicated process, involving many different aspects such as, transcription, translation, folding, post-translational modifications, secretion, etc., and many aspects of this process are still poorly understood. In addition, all of the genetic sequences (genes and other genetic elements) that control these various aspects have not been identified.

### SUMMARY OF THE INVENTION

The present invention is based on the identification of specific mammalian genes and sequences therein that are useful as RNAi targets to enhance the production of exogenous proteins in mammalian cell culture.

An RNAi target gene useful in the various aspects of the present invention is a gene that comprises any of the 343 RNAi Target Sequences in Table 1 below or an orthologous sequence present in any mammalian cell line that is suitable for producing an exogenous polypeptide in a large scale culture, e.g., Chinese hamster ovary (CHO) cells.

TABLE 1
RNAi Targets

| RNAi | SEQ ID | Mouse RNAi Target Gene |
|---|---|---|
| Target Sequence | NO. | (Accession No) |
| CCTGCAAGCTCCGCTTCTA | 1 | NM_024479 |
| CTTTGAAGACTTGAACAGT | 2 | NM_023697 |
| CCAAGATTGCCAAGCGCAT | 3 | NM_013523 |
| CATACCATTGGATGAGGTA | 4 | NM_029840 |
| CTCTGCATCTTCATCCGGA | 5 | NM_023168 |
| CCTACAAGCGCTTGTAGGA | 6 | NM_019980 |
| CTCAACTTCTGTGCCAATA | 7 | NM_013847 |
| GAAGCCCATTCCTCGAATT | 8 | NM_026871 |
| GAGCTAAACTCCACCAAGA | 9 | NM_133355 |
| CAGATACCACTTTCATCGA | 10 | NM_001003909 |
| CAAGTATGGCAGTATCATT | 11 | NM_025350 |
| CAGTATGTCTCCAACGACA | 12 | NM_025292 |
| GCGATATTATGAGCAACCT | 13 | NM_013415 |
| CTACACATCTTCTGGCAGA | 14 | NM_001081414 |
| GCATGGAGGCCTTCGGCAT | 15 | NM_133500 |
| GACATTGATTGCAATCCAT | 16 | NM_007958 |
| CTCATACCTTCTTTATTAT | 17 | NM_021509 |
| GAATGTATCTTCTGAAGGT | 18 | NM_177941 |
| CATTCAGGCACGGATGGAA | 19 | NM_080510 |
| GAACTAGAGCGGTTTGTAA | 20 | NM_001033156 |
| GTAATTGAGCTGTCAGTTT | 21 | NM_001081377 |
| CCATTTGTGCTGCAATAAT | 22 | NM_001014995 |
| GGACATATGTATTCGGGAA | 23 | NM_023543 |
| CATAGTAGAGTATGATGAT | 24 | NM_026009 |
| GAAACTTTACCCTTCTGAA | 25 | NM_133684 |
| CTCTAAGGGAGCTCTGTTT | 26 | NM_021491 |
| GTGATACCAGTGCAACTGA | 27 | NM_026984 |
| GACATGCGCTGGCTACGAA | 28 | NM_023131 |
| GGACTGGGTGCCTTACATC | 29 | NM_025650 |
| CACAGTTTGGAATTACAGA | 30 | NM_011217 |
| CTTGCCTGGTGACCGGGAA | 31 | NM_008048 |
| GCATTATGCGGACATTCAT | 32 | NM_025865 |
| CCTAGAAGATGAGCACCAA | 33 | NM_013855 |
| CCAGAAATTCCATAAGCCT | 34 | NM_025964 |
| CATTGACCAATGTTCGAGT | 35 | NM_001004761 |
| GAAACCAGGTCCACAGGAT | 36 | NM_019828 |
| GCAGTTACGACTTGGGCAA | 37 | NM_011521 |
| CACTGTATGACCTTCTGGT | 38 | NM_008737 |
| CATTCCATTGTTGTTGATT | 39 | NM_008770 |
| CATTCATCAGCAAGGTAAA | 40 | NM_001024385 |
| CAGAGTATGACGCCATGTT | 41 | NM_024267 |
| GGATCAACAGGGTGTTGTT | 42 | NM_008246 |
| CAATTACGTTTATAACACT | 43 | NM_024454 |
| CAGTTTAACTTCCAATCCA | 44 | NM_008626 |
| GTTTCGAAGTGCCTGTTTA | 45 | NM_007647 |
| GACTTATATCCAGGACAAA | 46 | NM_011169 |
| GCCAATAAATGACGTATTA | 47 | NM_025485 |
| CTCATGGTTCTCTTGAACT | 48 | NM_199314 |
| CTAACCACAGGTTCCGGAA | 49 | NM_008206 |
| CCTTATGGTGTCTTGCAAT | 50 | FJ042496 |
| CTTCCTTTAACAATCAGAT | 51 | NM_028052 |
| CTTGCAACCAGTTTGGCCA | 52 | NM_024198 |
| GCATGATTGTGGCCGAGGT | 53 | NM_027880 |
| CAGATAACCACCACGATGT | 54 | NM_028004 |
| GAAGGTACCTCCTGGTACA | 55 | NM_019688 |
| CACATTTCTGTTAAAGGTT | 56 | NM_028006 |
| GAGATGATCAGTTGGTACT | 57 | NM_027880 |
| CAGGAATCCTGGAGAGTAA | 58 | NM_133895 |
| GAATCTAGAGGATCTGGTT | 59 | NM_013563 |
| GAACAAGGTTGTCATGGAT | 60 | NM_013632 |
| CAGTATTGATGCAGTTTAT | 61 | NM_019724 |
| GCTACAAGACCCTGCCTTT | 62 | NM_013659 |
| CATACTCTATATCCATCTA | 63 | NM_177909 |
| CGGATAAGAACGAGAGGTT | 64 | NM_175638 |
| CGGATTTCAACTATTTCTT | 65 | NM_001081309 |
| CAACCAAGAAGAGGGCGAA | 66 | NM_011101 |
| CTTACAACTGGATTCAAGA | 67 | NM_029572 |
| GCAAGTATTTAGCATGAAT | 68 | NM_183294 |
| GCCGTAAGGTGTACGGCAT | 69 | NM_199029 |
| GTGCCTACAATAGCTGCTA | 70 | NM_023517 |
| GAAATCAATGCCTGAGGCA | 71 | NM_024236 |
| CTCATAAGAAGAGTTTCTT | 72 | NM_012048 |
| GCACTTTGACCTGAATCTA | 73 | NM_020603 |
| GGTTTATCCAGGAAGTATA | 74 | NM_008957 |
| GTTATCATGTGGGAAGTGT | 75 | NM_138952 |
| GGAGTTTATACCAAACAGA | 76 | NM_001001321 |
| CAAGTTCTGTGACGAGTGT | 77 | NM_010588 |
| GAAGTTTGAAGGCTGTTAT | 78 | NM_172844 |
| AGGATATTCACTTTCATAT | 79 | NM_028450 |
| GACATCACCTCTGTTCTTC | 80 | NM_030037 |
| GATGTCAACAGAGCCATCA | 81 | NM_028475 |
| CTGTTGCCTGCTTACATTT | 82 | NM_019773 |
| GCTACAAGCAGTCCTTGAA | 83 | NM_178076 |
| GGATGATGAAGGTGTTCTT | 84 | NM_028784 |
| CAATAAAGAAGATATAGTT | 85 | NM_018852 |
| CTGTGTTGCCAATAGGGAA | 86 | NM_007979 |
| GAATGTACCTGTCATGAAT | 87 | NM_001037755 |
| CCTCTATACCTCCATCTTT | 88 | NM_173388 |
| GATTGCATGGCTGTTCAAT | 89 | NM_133216 |
| GAATCTCGAAGCTTATATA | 90 | NM_029947 |
| GTCAAGACCTGGTATGAGT | 91 | NM_001195413 |
| CAGATCATTGTTCATGCTT | 92 | NM_001110327 |
| GAGATGTGGCCCTTCTTCA | 93 | NM_019791 |
| GCTCAAAGTGTGTAAGTTT | 94 | NM_028175 |
| CCAAATATGGCTTGTACTT | 95 | NM_028950 |
| CCTGGAATGTCTTCATTAT | 96 | NM_021426 |
| GACATTCAGAGCCGAAGTA | 97 | NM_033524 |
| GTGTTTGTGGAACTGCTTA | 98 | NM_013815 |
| CTCAACATGTCTCGGTGTT | 99 | NM_028105 |
| CTTGGTACTTGCTACGTGT | 100 | NM_001014976 |
| CAGCTAGCATCGGGACAGA | 101 | NM_053208 |
| CATGTTCTATTGGATGTAT | 102 | NM_026209 |
| GCACTGTTCCGCAAGGGCA | 103 | NM_001111066 |
| GGCAGTTCGACTATGCGTT | 104 | NM_183315 |
| CCAAGTTTCATCTTATGCA | 105 | NM_134109 |
| GAACTTTAAGGATATTACA | 106 | NM_019972 |
| GAAGCATTATCCAGATCTA | 107 | NM_030696 |
| CTGCTCTTCTGCAGGTGGA | 108 | NM_178682 |
| GAAATGAGTCCACAGATGA | 109 | NM_146164 |
| GGAGTATCATCTTGGGCTA | 110 | NM_010484 |
| CGATTTGGATAGCCAACAA | 111 | NM_010113 |
| CCACTTACTTCATGACCTA | 112 | NM_008720 |
| CACTAATTCAGTTCCAGTA | 113 | NM_029094 |
| GACAGTACCCAGCGACTTT | 114 | NM_029097 |
| CACAGTGCTACTTCGAGTA | 115 | NM_030558 |
| CAAGGAATTATCATTCAGA | 116 | NM_001039094 |
| GTGAGTGTGTGATCTGCCT | 117 | NM_133206 |
| GCAACAGTGGCAGTGGGAA | 118 | NM_010442 |
| GAATTACAAGGGCTACTTT | 119 | NM_001080926 |
| GCCTTCAAGGCGGAGATCA | 120 | NM_010402 |
| CTACCTAGCTGACGAGATT | 121 | NM_010602 |
| GGAATTCATGTCTAAGTCT | 122 | NM_016781 |
| GAAATGCCTTTCAATGTCA | 123 | NM_011454 |
| CTCTATGGGTTCCTGGTTA | 124 | NM_021607 |
| CAGATTACTCCTGGATGAA | 125 | NM_029077 |
| GCATTCCATTGTTGTTGAT | 126 | NM_008770 |
| GTTATGGTCCTCATTCTTA | 127 | NM_021339 |
| CCACAATTGTTGATGTGAA | 128 | NM_029582 |
| GATGAAGATTGAGGAGTAT | 129 | NM_027328 |
| GACAAATTGGATATTATTT | 130 | NM_010111 |
| GAATTGAGCCACAGAGTAA | 131 | NM_010754 |
| GAAACTGAAGGCTTTCTTA | 132 | NM_027838 |
| GAACTACACATCTAAGTTT | 133 | NM_028186 |
| CACAGTTCATCTTCGACCA | 134 | NM_010413 |
| CAAAGAAGTTTGCAGATTT | 135 | NM_026769 |
| CGAATGAAGGATTTCACCA | 136 | NM_027088 |
| CCTTTGAGCTGGGCGAGAT | 137 | NM_018810 |
| CTTTACCTGGAATATTACA | 138 | NM_013880 |
| GTTCTTGTGACTCATTAGT | 139 | NM_008133 |
| CACAGAACCTCTTGTTGGA | 140 | NM_019827 |
| GTTAGAAATGGCTTCCGGA | 141 | NM_009929 |
| GTCGTTATGGAACTCCTGA | 142 | NM_028803 |
| GGATCTTTGGCTGGGCAGA | 143 | NM_029880 |
| CTTTGACAACTGTTTACGA | 144 | NM_016792 |
| GGAGTTAATCTCTTCGACA | 145 | NM_010597 |
| CCATAGTCCTCTCCAAATT | 146 | NM_010865 |
| GAATGACCCTGTCACTTTA | 147 | NM_008860 |
| GGTACTTTCAGTTTCCAGA | 148 | XM_006502607 |
| CCGATTCTTGTATCATGGA | 149 | NM_026536 |
| GCAAGTTGGTCAGACAGGA | 150 | NM_145615 |
| GACGTATGGCTCAAATGGT | 151 | NM_013630 |
| CACTACTTCATCGAGGTCA | 152 | NM_008797 |
| CTAACATGCTGAATAGGTA | 153 | NM_008171 |
| CAGATCTGTTTGAACGGAA | 154 | NM_173185 |
| CCAAGTGTTCTTCAGGCAA | 155 | NM_008842 |
| GTCATTAGAGTTGCCCAGA | 156 | NM_134438 |
| TCTAGAGTGCCAATCATA | 157 | NM_080644 |
| CTCATTTAAGGTACCCTTT | 158 | NM_007513 |
| CACTTGGATTGGCCTAACT | 159 | NM_009714 |
| CACATCAAGTTCACCCAGA | 160 | NM_007835 |
| CATGGATTCTTCACTCGTT | 161 | NM_007659 |
| GTCATTGGGCTGCTCTATA | 162 | NM_145440 |
| GCAATGTGTTCTTATGGAA | 163 | NM_133728 |
| GAATTATGGGACGATTATT | 164 | NM_001113352 |
| GGAAGTATTTCACATGTGA | 165 | NM_007835 |
| GAGAGTCCTGCCGGTGGTA | 166 | NM_007878 |
| CAATGACTGCTCTTGTGCA | 167 | NM_007611 |
| CATGCTATGAAGTACTCCA | 168 | NM_008032 |
| CTATGATCCTGCCAAACGA | 169 | NM_007659 |
| CCCTTTATCATTGAGTGGA | 170 | NM_146170 |
| CACAGAAACCTTCCAGGTA | 171 | NM_011352 |
| GCTGTTATGGATTAATTGT | 172 | NM_008772 |
| GAGGAGTTTCCATCACGAA | 173 | NM_007554 |
| GAGAAACGTCCTTTCATGT | 174 | NM_144783 |
| CACACTTCTTCAAGGTGCT | 175 | NM_007931 |
| GCTAGAAGATGGGACGGAG | 176 | NM_008020 |
| CAAGAAGGATTTGACCGTA | 177 | NM_008792 |
| CAGATCATCTTTGCAGTGA | 178 | NM_134160 |
| CTCATTCATTTCCCAGTGT | 179 | NM_134066 |
| CATCCAAGTGGATTGCTTT | 180 | NM_001110204 |
| CTACACTGGCCGTTTCTTA | 181 | NM_008277 |
| CTATCCAGATGGTCGCCAA | 182 | NM_001110208 |
| CTGCTATTTCCATCCATGA | 183 | NM_008832 |
| CCTATACCTTCAATTGCCA | 184 | NM_030688 |
| CGATTTGCCACCAAAGCTT | 185 | NM_008426 |
| CATCGAGCCTGACATCAAA | 186 | NM_030677 |
| CCAACTATGGCATCGTCAC | 187 | NM_023057 |
| GAGATCTACCGATACACAT | 188 | NM_009993 |
| GTTCGAAGGCTGGTGGGAA | 189 | NM_172903 |
| GCATTGACCGCTGTGTCAA | 190 | NM_145542 |
| CCAACAAGGAGGAACTCAT | 191 | NM_028882 |
| CAGAATTACTTAATAGAGA | 192 | NM_026719 |
| CTATGTGTGTGATCTTGAA | 193 | NM_010924 |
| GAAAGTTCCTGCCAGCAGT | 194 | NM_134028 |
| CTACCAAACTTCGAGGATA | 195 | NM_054085 |
| GAGCTGACATGCTCATGGT | 196 | NM_008525 |
| CCTTCAAGAGGGCCACCTA | 197 | NM_173071 |
| GTATTGATAAGCAACGTGA | 198 | NM_029706 |
| CTTATGAGCTGGTGATCGA | 199 | NM_001098170 |
| GCAATTGTAACTTACCCTT | 200 | NM_178749 |
| GCCATTATTCAGTGATGTT | 201 | NM_145458 |
| CGATTCAACATTAACGATA | 202 | NM_001105196 |
| GTCGAATTGAACATTTATA | 203 | NM_146041 |
| GATACCTTCTGGGTTCAGA | 204 | NM_026482 |
| CTCGTTTGCCTTAATTCTT | 205 | NM_145608 |
| GAGAGAAACTTGATGGGAA | 206 | NM_144954 |
| CTAACAACCGGCTCCTCAA | 207 | NM_178907 |
| GTCTCTAACACCATCGGAA | 208 | NM_172152 |
| GAGATGATGATTGGATTCA | 209 | NM_148931 |
| CATTGAGGACCTTCGTGTA | 210 | NM_177185 |
| GAAGGATTGTGGTTATGGA | 211 | NM_175540 |
| GGAGTCTGGTTTAACCTCA | 212 | NM_008437 |
| GCTACATGACCGATGGACT | 213 | NM_026406 |
| GCAAGTTTGTGCCCTCAGA | 214 | NM_146177 |
| CTGACAAAGAGCTTCCACT | 215 | NM_144893 |
| CCCATAACGGAGAATCTGT | 216 | NM_029294 |
| CCAATGATGGCCATTCAGT | 217 | NM_178396 |
| CCTTCAACATTGACCGAAA | 218 | NM_145922 |
| CATTCGAAACCACAAATCT | 219 | NM_152804 |
| GAAATACTGTCTCCCTTTA | 220 | NM_212433 |
| GAAATACTACCATCTTACT | 221 | NM_001110497 |
| GGTGTAAATTAAGTAGCAA | 222 | NM_021516 |
| CTTACTCTTCTCACATGGA | 223 | NM_146096 |
| CTATTGGATTTACCATGTA | 224 | NM_008174 |
| GAGATGGTGTACTGCTACA | 225 | NM_011322 |
| GTACTTCCGAGAACTCTTT | 226 | NM_008846 |
| GCAGTATGTGGCCAGTTAT | 227 | NM_028850 |
| CAGCATTTGTCAAACGTCA | 228 | NM_173379 |
| GTATGAAACGGCTAGAATT | 229 | NM_133819 |
| CTCAGCAGTGGATGACCAA | 230 | NM_033573 |
| CCTTCTTCCTCTCCATGTA | 231 | NM_139142 |
| CTCTTGGACTACCTTATGA | 232 | NM_177357 |
| CTTTGGACCTCGACTCATT | 233 | NM_010957 |
| CTCAGAATTTGCTGACCCT | 234 | NM_009469 |
| GATGTCAAGTCATATTACA | 235 | NM_011201 |
| GATAGTTTATTGGGTGCTT | 236 | NM_178746 |
| CAAGAAGAAGCGTTTCTGA | 237 | NM_008518 |
| GCTTTCAACTGTATGGTCT | 238 | NM_029020 |
| GATACTGGGTGTTTAAGGA | 239 | NM_011846 |
| GGAATTGTACCTCGATCTT | 240 | NM_144792 |
| CTTGTAAGTGGGTCACCAA | 241 | NM_177725 |
| GAAATATATAGCTTTGGAA | 242 | NM_029005 |
| CACTGTACCTCTAATTACA | 243 | NM_198246 |
| CCTATTGGAATCTGGGCTA | 244 | NM_033269 |
| TGTTTATGACCCTGGCTTA | 245 | NM_019513 |
| CCAAAGATGGCATTGACCA | 246 | NM_148930 |
| GTTTGCATCCGGCACCTGA | 247 | NM_198214 |
| GTCTATGTCTTCATTCAGA | 248 | NM_001029842 |
| CATGGATTTGGATGTAGTA | 249 | NM_009157 |
| CCAATTGGACTTAACATGT | 250 | NM_146003 |
| CCTTCACTATGGTGCGCTA | 251 | NM_029536 |
| GAGAACTCATGGTTTAACT | 252 | NM_018736 |
| GGAGATTTCTCACGATGAA | 253 | NM_001081206 |
| CATTTGCTATGAACACGCA | 254 | NM_021360 |
| GCCTTATTATGATCTTGTA | 255 | NM_009370 |
| CTGTTCAAGAAAGAGGTGA | 256 | NM_013571 |
| GAATTCTACTTCCCAACAT | 257 | NM_001081306 |
| CAGGTTACATATCAGTTGA | 258 | NM_144902 |
| CCACCTATCCCTCTGGACA | 259 | NM_016689 |
| CCAAGGAGCAGCGACTCAA | 260 | NM_019719 |
| CACCTTTGAGCAATGGTCT | 261 | NM_019653 |
| GCCAATATAGATCTGCTTA | 262 | NM_016697 |
| GAGATTGGACTCTGCAGTA | 263 | NM_175025 |
| GGAACTTAAGGAAACCTTT | 264 | NM_016708 |
| GTATAATGCTAAAGACACA | 265 | NM_001081315 |
| GTGCATTTCGGGTAGGGAT | 266 | NM_013540 |
| CATTGACAGATATTGGGCA | 267 | NM_145700 |
| CTCAAAGGAGGTCATGTTT | 268 | NM_009097 |
| GGATATTTGCCACAGCATT | 269 | NM_145355 |
| CATTGAGTACTCTGGCATT | 270 | NM_029274 |
| GAATTGGTGGCAGAACAGA | 271 | NM_019802 |
| GCTCTTACCAGACAATCAA | 272 | NM_153792 |
| CTAATGAACAGGTGTTGAA | 273 | NM_010568 |
| CTACCTCGTGGGATCCCAT | 274 | NM_175087 |
| CGCTCTACTTCTGCTTCGT | 275 | NM_199251 |
| CTAAGAACATGATGTGGTA | 276 | NM_177646 |
| CAGTCAAAGACATATGAGA | 277 | NM_146086 |
| CTCAATGGAGGACTTGAAT | 278 | NM_146239 |
| GATTATACCTTAACTATGT | 279 | NM_001038701 |
| CCATGCTAGTGGTCCTCAT | 280 | NM_144834 |
| CTCATCAAATCCCGAGGGA | 281 | NM_145121 |
| GACACTATAGTCGAGCCAA | 282 | NM_019827 |
| CCTTAAGTCCAGCAACATA | 283 | NM_177395 |
| GAGGAAATTGCCAGGATCA | 284 | NM_175127 |
| GTCTCAAGGTACTTTACGA | 285 | NM_021390 |
| GGTTATACAGGTTGTTAAA | 286 | NM_138606 |
| CCTATTGGATCTCCCAGCT | 287 | NM_001109045 |
| GATCTTCGGAACTTAGTGA | 288 | NM_016744 |
| CCATTAACCAGAGAATAGT | 289 | NM_010154 |
| CTCGAAAGCAAAGAGATAA | 290 | NM_009595 |
| GGAACATTCTAGAATCTGT | 291 | NM_021485 |
| CACATTGAGAGAGTGTATA | 292 | NM_199446 |
| CCATCATTTGTATTCCGGA | 293 | NM_011083 |
| CAGTTCAAGGCTGCGCTCA | 294 | NM_001033254 |
| GGATTACAACCTGAGAGTT | 295 | NM_153135 |
| CTTGTTGCCTCACATCCGA | 296 | NM_011903 |
| GAAATTATTGGGCGTCATA | 297 | NM_001033209 |
| CCATCAAGCAGGGTTTCAA | 298 | NM_213733 |
| GGAAATGATGACTTTATGT | 299 | NM_023209 |
| GACAATTCAGAGAGTCACA | 300 | NM_207683 |
| CCAATAATGTCACCTCGAA | 301 | NM_010899 |
| GACCTAGATGTCATTGTTT | 302 | NM_011658 |
| GAAGTTTGACCGCTACAAA | 303 | NM_011716 |
| GTGTATGCCGCAACGGCTA | 304 | NM_010142 |
| CAGCTAATCATCAACCCAA | 305 | NM_010934 |
| CAATTTGAACCAAGAGTCT | 306 | NM_011815 |
| CAGATACCGGGCCCAACGA | 307 | NM_018810 |
| CAGTTCTCGATCACCGGTA | 308 | NM_133882 |
| GGATCAACCTTCCTATCCA | 309 | NM_010043 |
| CATATGATCTCCACCATGA | 310 | NM_001033328 |
| GTATGTGCGCTATTTCCGA | 311 | NM_177648 |
| CAATACAGTTCCGCTTGTA | 312 | NM_010434 |
| GTGAATCTCAACTACCGAA | 313 | NM_011706 |
| GGAATACCTTTCATCGATT | 314 | NM_028779 |
| GACCTATGATGCACCACAT | 315 | NM_175514 |
| GGCAACAGTTCTTCATCGA | 316 | NM_008381 |
| CCGGAAATACAGATTGATT | 317 | NM_021565 |
| CGCGAGGTGCACTTCATGA | 318 | NM_010205 |
| CCATGAACGCCGAGGACGA | 319 | NM_010608 |
| CATTCTAAAGGTCAGTGTA | 320 | NM_010728 |
| GATCATCTGCCTTCCGAAA | 321 | NM_133897 |
| CCTACTTTGGCCTGCTTGA | 322 | NM_025968 |
| CTTACTACAACAATCTGTA | 323 | NM_015826 |
| GGAATATCTTCTAATGGGA | 324 | NM_009011 |
| CGTGTTAGCCTTACACCAA | 325 | NM_025846 |
| GTAATTGAGTATAAGTCCA | 326 | NM_011218 |
| GTTTGACATTGAGATTGGA | 327 | NM_175638 |
| CAGACTTCTTTGAGTACTT | 328 | NM_010794 |
| CAGTGAATCAGGTGGCGAA | 329 | NM_177992 |
| GGGATAAGCCTCCTTTCTT | 330 | NM_175465 |
| CACAATGTGGTCCACATGA | 331 | NM_207210 |
| GTACCTCTCCTATAGACAT | 332 | NM_009809 |
| CGGTATTGATGATGTGCCT | 333 | NM_001008702 |
| CTTGAACTTCCACGAGGTA | 334 | NM_011846 |
| CATAAGATTGGCTGGAATT | 335 | NM_177353 |
| GCACCTACCTGGGTCACTT | 336 | NM_198108 |
| CTTAGACTGGAAATTAAGA | 337 | NM_011077 |
| GAGCTAAAGCTGTAACCCA | 338 | NM_021516 |
| GGTGATAGGTAAGAATATT | 339 | NM_199159 |
| GAGATGATGGACTCATGTA | 340 | NM_023383 |
| CACCTTATATCCCAAATGT | 341 | NM_001037758 |
| CCTACGTCTTCAACATCAT | 342 | NM_016975 |
| CATCAAGCAGGGTTTCAAA | 343 | NM_213733 |

In some embodiments, the RNAi target gene is a mammalian gene that comprises any of the 75 RNAi Target Sequences in Table 2 below, or an orthologous sequence present in any mammalian cell line that is suitable for producing an exogenous polypeptide in large scale culture, e.g., Chinese hamster ovary (CHO) cells.

TABLE 2
RNAi Targets; Subset 1

| | | Mouse RNAi | CHO RNAi |
|---|---|---|---|
| | SEQ | Target Gene | Target Gene |
| RNAi Target | ID | (Accession | (Accession |
| Sequence | NO | No.) | No.) |
| GAACTAGAGCGGTTTGTAA | 20 | NM_001033156 | XM_003511078 |
| CTTCCTTTAACAATCAGAT | 51 | NM_028052 | XM_003503668.1 |
| GACTTATATCCAGGACAAA | 46 | NM_011169 | XM_003504158 |
| GGATCAACAGGGTGTTGTT | 42 | NM_008246 | XM_003513214 |
| GGTTTATCCAGGAAGTATA | 74 | NM_008957 | XM_003514770 |
| CAGGAATCCTGGAGAGTAA | 58 | NM_133895 | XM_003505358 |
| GGAGTATCATCTTGGGCTA | 110 | NM_010484 | XM_003506082 |
| GTGAGTGTGTGATCTGCCT | 117 | NM_133206 | XM_003510170 |
| GGATGATGAAGGTGTTCTT | 84 | NM_028784 | XM_003497479 |
| CGATTTGGATAGCCAACAA | 111 | NM_010113 | XM_007653160 |
| CCAAATATGGCTTGTACTT | 95 | NM_028950 | XM_003503382 |
| CCACTTACTTCATGACCTA | 112 | NM_008720 | NM_001246687 |
| GATTGCATGGCTGTTCAAT | 89 | NM_133216 | XM_003511942 |
| CAGCTAGCATCGGGACAGA | 101 | NM_053208 | XM_003505261 |
| GAATTGAGCCACAGAGTAA | 131 | NM_010754 | XM_003501037, |
| | | | XM_003501038 |
| GAAACTGAAGGCTTTCTTA | 132 | NM_027838 | XR_135851 |
| GAACTACACATCTAAGTTT | 133 | NM_028186 | XM_003515516 |
| CACAGTTCATCTTCGACCA | 134 | NM_010413 | XM_003505101 |
| CAAAGAAGTTTGCAGATTT | 135 | NM_026769 | XM_003512003 |
| CAAGAAGGATTTGACCGTA | 177 | NM_008792 | XM_003508113 |
| CTATCCAGATGGTCGCCAA | 182 | NM_001110208 | XM_003505253 |
| CCCTTTATCATTGAGTGGA | 170 | NM_146170 | XM_003507962, |
| | | | XM_003507963 |
| CTGCTATTTCCATCCATGA | 183 | NM_008832 | XM_003512672, |
| | | | XM_003512673 |
| CCTATACCTTCAATTGCCA | 184 | NM_030688 | XM_003507545 |
| TCTAGAGTGCCAATCATA | 157 | NM_080644 | NW_006879584 |
| GAGATCTACCGATACACAT | 188 | NM_009993 | XM_003502400 |
| CCGATTCTTGTATCATGGA | 149 | NM_026536 | XM_003511591 |
| CTTATGAGCTGGTGATCGA | 199 | NM_001098170 | XM_003495474 |
| CTATGTGTGTGATCTTGAA | 193 | NM_010924 | XM_003509317 |
| GGAGTCTGGTTTAACCTCA | 212 | NM_008437 | XM_003510897 |
| CTACCAAACTTCGAGGATA | 195 | NM_054085 | XM_003511682 |
| CATTGAGGACCTTCGTGTA | 210 | NM_177185 | XM_003503221 |
| CCAATGATGGCCATTCAGT | 217 | NM_178396 | XM_003499339 |
| CTAACAACCGGCTCCTCAA | 207 | NM_178907 | XM_003500390 |
| CCTTCAACATTGACCGAAA | 218 | NM_145922 | XM_003514579 |
| GCCATTATTCAGTGATGTT | 201 | NM_145458 | XM_003502752 |
| GCAAGTTTGTGCCCTCAGA | 214 | NM_146177 | XM_003508241 |
| CTGACAAAGAGCTTCCACT | 215 | NM_144893 | NM_001243991 |
| CACTGTACCTCTAATTACA | 243 | NM_198246 | XM_003513347 |
| GCTTTCAACTGTATGGTCT | 238 | NM_029020 | XM_003501302 |
| CTCAGCAGTGGATGACCAA | 230 | NM_033573 | XM_003502900 |
| GCAGTATGTGGCCAGTTAT | 227 | NM_028850 | XM_003513492 |
| CCTTCTTCCTCTCCATGTA | 231 | NM_139142 | XM_003507723 |
| GATGTCAAGTCATATTACA | 235 | NM_011201 | XM_003502483 |
| CTTACTCTTCTCACATGGA | 223 | NM_146096 | XM_003511733 |
| CAGCATTTGTCAAACGTCA | 228 | NM_173379 | XM_007642431 |
| CTTGTAAGTGGGTCACCAA | 241 | NM_177725 | XM_003513970 |
| GGAGATTTCTCACGATGAA | 253 | NM_001081206 | XM_003500967 |
| GGATATTTGCCACAGCATT | 269 | NM_145355 | XM_003509299 |
| CCAAAGATGGCATTGACCA | 246 | NM_148930 | XM_003500410 |
| GTGCATTTCGGGTAGGGAT | 266 | NM_013540 | XM_003500211, |
| | | | XM_003500212 |
| GCCAATATAGATCTGCTTA | 262 | NM_016697 | XM_007643638 |
| CAGTCAAAGACATATGAGA | 277 | NM_146086 | XM_003506313 |
| CCATGCTAGTGGTCCTCAT | 280 | NM_144834 | XM_003509378 |
| GCTCTTACCAGACAATCAA | 272 | NM_153792 | XM_003501507, |
| | | | XM_003501508 |
| GGAACATTCTAGAATCTGT | 291 | NM_021485 | XM_003509935 |
| GGTTATACAGGTTGTTAAA | 286 | NM_138606 | XM_003505096 |
| CCATCATTTGTATTCCGGA | 293 | NM_011083 | XM_003504810 |
| CTCGAAAGCAAAGAGATAA | 290 | NM_009595 | XM_003507470, |
| | | | XM_003507471 |
| CTAAGAACATGATGTGGTA | 276 | NM_177646 | XM_003512693 |
| CCTATTGGATCTCCCAGCT | 287 | NM_001109045 | XM_003514321 |
| GACCTATGATGCACCACAT | 315 | NM_175514 | XM_003503395 |
| CATATGATCTCCACCATGA | 310 | NM_001033328 | XM_0076535101 |
| GATCATCTGCCTTCCGAAA | 321 | NM_133897 | XM_003506392 |
| CCTACTTTGGCCTGCTTGA | 322 | NM_025968 | XM_003510575 |
| GTATGTGCGCTATTTCCGA | 311 | NM_177648 | XM_003513968 |
| GTGTATGCCGCAACGGCTA | 304 | NM_010142 | XM_003503638 |
| CAATACAGTTCCGCTTGTA | 312 | NM_010434 | XM_003497435, |
| | | | XM_003497436 |
| CAGCTAATCATCAACCCAA | 305 | NM_010934 | XM_003510777 |
| GTGAATCTCAACTACCGAA | 313 | NM_011706 | XM_003496342, |
| | | | XM_003496343 |
| CTTACTACAACAATCTGTA | 323 | NM_015826 | XM_003514815 |
| CGGTATTGATGATGTGCCT | 333 | NM_001008702 | XM_003503098 |
| CCTACGTCTTCAACATCAT | 342 | NM_016975 | XM_003500430 |
| GGTGATAGGTAAGAATATT | 339 | NM_199159 | XM_003504375 |
| GTTTGACATTGAGATTGGA | 327 | NM_175638 | XM_003504666 |

In other embodiments, an RNAi target gene is a mammalian gene that comprises any of the 11 RNAi Target Sequences in Table 3 below or an orthologous sequence present in any mammalian cell line that is useful for producing exogenous polypeptides.

TABLE 3
RNAi Targets, Subset 2

| | | Mouse RNAi | CHO RNAi |
|---|---|---|---|
| | SEQ | Target Gene | Target Gene |
| RNAi Target | ID | (Accession | (Accession |
| Sequence | NO | No.) | No.) |
| GAGATCTACCGATACACAT | 188 | NM_009993 | XM_003502400 |
| CCGATTCTTGTATCATGGA | 149 | NM_026536 | XM_003511591 |
| CATTGAGGACCTTCGTGTA | 210 | NM_177185 | XM_003503221 |
| CAGCATTTGTCAAACGTCA | 228 | NM_173379 | XM_007642431 |
| GGAGATTTCTCACGATGAA | 253 | NM_001081206 | XM_003500967 |
| GGATATTTGCCACAGCATT | 269 | NM_145355 | XM_003509299 |
| GCCAATATAGATCTGCTTA | 262 | NM_016697 | XM_007643638 |
| GCTCTTACCAGACAATCAA | 272 | NM_153792 | XM_003501507, |
| | | | XM_003501508 |
| GGAACATTCTAGAATCTGT | 291 | NM_021485 | XM_003509935 |
| GGTGATAGGTAAGAATATT | 339 | NM_199159 | XM_003504375 |
| GTTTGACATTGAGATTGGA | 327 | NM_175638 | XM_003504666 |

Thus, in one aspect, the present invention provides a method of producing a polypeptide, which comprises providing a recombinant mammalian host cell capable of expressing the polypeptide, culturing the host cell under conditions suitable for effecting expression of the polypeptide and inhibiting expression of at least one RNAi target gene selected from the group of mammalian genes listed in Table 1, 2 or 3 or an ortholog thereof, and recovering the expressed polypeptide.

In some embodiments, expression of the RNAi target gene is inhibited by transfecting the host cell with a short interfering nucleic acid (siNA) molecule that is capable of inhibiting expression of the selected RNAi target gene(s). The siNA is preferably a short interfering RNA (siRNA) molecule selected from the group of siRNAs listed in Table 4 below. More preferably, the siNA molecule comprises the antisense and sense sequence pair shown in Table 4 for an RNAi Target Sequence shown in Table 3.

TABLE 4
Exemplary siRNA molecules of the Invention

| RNAi | | |
|---|---|---|
| Target | | |
| Se- | | |
| quence | | |
| (Table | siRNA | |
| 2) | ANTISENSE SEQUENCE | SENSE SEQUENCE |
| 20 | UUACAAACCGCUCUAGUUCTT | GAACUAGAGCGGUUUGUAATT |
| | (SEQ ID NO: 344) | (SEQ ID NO: 345) |
| 51 | AUCUGAUUGUUAAAGGAAGTT | CUUCCUUUAACAAUCAGAUTT |
| | (SEQ ID NO: 346) | (SEQ ID NO: 347) |
| 46 | UUUGUCCUGGAUAUAAGUCTT | GACUUAUAUCCAGGACAAATT |
| | (SEQ ID NO: 348) | (SEQ ID NO: 349) |
| 42 | AACAACACCCUGUUGAUCCTT | GGAUCAACAGGGUGUUGUUTT |
| | (SEQ ID NO: 350) | (SEQ ID NO: 351) |
| 74 | UAUACUUCCUGGAUAAACCTT | GGUUUAUCCAGGAAGUAUATT |
| | (SEQ ID NO: 352) | (SEQ ID NO: 353) |
| 58 | UUACUCUCCAGGAUUCCUGTT | CAGGAAUCCUGGAGAGUAATT |
| | (SEQ ID NO: 354) | (SEQ ID NO: 355) |
| 110 | UAGCCCAAGAUGAUACUCCTT | GGAGUAUCAUCUUGGGCUATT |
| | (SEQ ID NO: 356) | (SEQ ID NO: 357) |
| 117 | AGGCAGAUCACACACUCACTT | GUGAGUGUGUGAUCUGCCUTT |
| | (SEQ ID NO: 358) | (SEQ ID NO: 359) |
| 84 | AAGAACACCUUCAUCAUCCTT | GGAUGAUGAAGGUGUUCUUTT |
| | (SEQ ID NO: 360) | (SEQ ID NO: 361) |
| 111 | UUGUUGGCUAUCCAAAUCGTT | CGAUUUGGAUAGCCAACAATT |
| | (SEQ ID NO: 362) | (SEQ ID NO: 363) |
| 95 | AAGUACAAGCCAUAUUUGGTT | CCAAAUAUGGCUUGUACUUTT |
| | (SEQ ID NO: 364) | (SEQ ID NO: 365) |
| 112 | UAGGUCAUGAAGUAAGUGGTT | CCACUUACUUCAUGACCUATT |
| | (SEQ ID NO: 366) | (SEQ ID NO: 367) |
| 89 | AUUGAACAGCCAUGCAAUCTT | GAUUGCAUGGCUGUUCAAUTT |
| | (SEQ ID NO: 368) | (SEQ ID NO: 369) |
| 101 | UCUGUCCCGAUGCUAGCUGTT | CAGCUAGCAUCGGGACAGATT |
| | (SEQ ID NO: 370) | (SEQ ID NO: 371) |
| 131 | UUACUCUGUGGCUCAAUUCTT | GAAUUGAGCCACAGAGUAATT |
| | (SEQ ID NO: 372) | (SEQ ID NO: 373) |
| 132 | UAAGAAAGCCUUCAGUUUCTT | GAAACUGAAGGCUUUCUUATT |
| | (SEQ ID NO: 374) | (SEQ ID NO: 375) |
| 133 | AAACUUAGAUGUGUAGUUCTT | GAACUACACAUCUAAGUUUTT |
| | (SEQ ID NO: 376) | (SEQ ID NO: 377) |
| 134 | UGGUCGAAGAUGAACUGUGTT | CACAGUUCAUCUUCGACCATT |
| | (SEQ ID NO: 378) | (SEQ ID NO: 379) |
| 135 | AAAUCUGCAAACUUCUUUGTT | CAAAGAAGUUUGCAGAUUUTT |
| | (SEQ ID NO: 380) | (SEQ ID NO: 381) |
| 177 | UACGGUCAAAUCCUUCUUGTT | CAAGAAGGAUUUGACCGUATT |
| | (SEQ ID NO: 382) | (SEQ ID NO: 383) |
| 182 | UUGGCGACCAUCUGGAUAGTT | CUAUCCAGAUGGUCGCCAATT |
| | (SEQ ID NO: 384) | (SEQ ID NO: 385) |
| 170 | UCCACUCAAUGAUAAAGGGTT | CCCUUUAUCAUUGAGUGGATT |
| | (SEQ ID NO: 386) | (SEQ ID NO: 387) |
| 183 | UCAUGGAUGGAAAUAGCAGTT | CUGCUAUUUCCAUCCAUGATT |
| | (SEQ ID NO: 388) | (SEQ ID NO: 389) |
| 184 | UGGCAAUUGAAGGUAUAGGTT | CCUAUACCUUCAAUUGCCATT |
| | (SEQ ID NO: 390) | (SEQ ID NO: 391) |
| 157 | UAUGAUUGGCACUCUAGAGTT | CUCUAGAGUGCCAAUCAUATT |
| | (SEQ ID NO: 392) | (SEQ ID NO: 393) |
| 188 | AUGUGUAUCGGUAGAUCUCTT | GAGAUCUACCGAUACACAUTT |
| | (SEQ ID NO: 394) | (SEQ ID NO: 395) |
| 149 | UCCAUGAUACAAGAAUCGGTT | CCGAUUCUUGUAUCAUGGATT |
| | (SEQ ID NO: 396) | (SEQ ID NO: 397) |
| 199 | UCGAUCACCAGCUCAUAAGTT | CUUAUGAGCUGGUGAUCGATT |
| | (SEQ ID NO: 398) | (SEQ ID NO: 399) |
| 193 | UUCAAGAUCACACACAUAGTT | CUAUGUGUGUGAUCUUGAATT |
| | (SEQ ID NO: 400) | (SEQ ID NO: 401) |
| 212 | UGAGGUUAAACCAGACUCCTT | GGAGUCUGGUUUAACCUCATT |
| | (SEQ ID NO: 402) | (SEQ ID NO: 403) |
| 195 | UAUCCUCGAAGUUUGGUAGTT | CUACCAAACUUCGAGGAUATT |
| | (SEQ ID NO: 404) | (SEQ ID NO: 405) |
| 210 | UACACGAAGGUCCUCAAUGTT | CAUUGAGGACCUUCGUGUATT |
| | (SEQ ID NO: 406) | (SEQ ID NO: 407) |
| 217 | ACUGAAUGGCCAUCAUUGGTT | CCAAUGAUGGCCAUUCAGUTT |
| | (SEQ ID NO: 408) | (SEQ ID NO: 409) |
| 207 | UUGAGGAGCCGGUUGUUAGTT | CUAACAACCGGCUCCUCAATT |
| | (SEQ ID NO: 410) | (SEQ ID NO: 411) |
| 218 | UUUCGGUCAAUGUUGAAGGTT | CCUUCAACAUUGACCGAAATT |
| | (SEQ ID NO: 412) | (SEQ ID NO: 413) |
| 201 | AACAUCACUGAAUAAUGGCTT | GCCAUUAUUCAGUGAUGUUTT |
| | (SEQ ID NO: 414) | (SEQ ID NO: 415) |
| 214 | UCUGAGGGCACAAACUUGCTT | GCAAGUUUGUGCCCUCAGATT |
| | (SEQ ID NO: 416) | (SEQ ID NO: 417) |
| 215 | AGUGGAAGCUCUUUGUCAGTT | CUGACAAAGAGCUUCCACUTT |
| | (SEQ ID NO: 418) | (SEQ ID NO: 419) |
| 243 | UGUAAUUAGAGGUACAGUGTT | CACUGUACCUCUAAUUACATT |
| | (SEQ ID NO: 420) | (SEQ ID NO: 421) |
| 238 | AGACCAUACAGUUGAAAGCTT | GCUUUCAACUGUAUGGUCUTT |
| | (SEQ ID NO: 422) | (SEQ ID NO: 423) |
| 230 | UUGGUCAUCCACUGCUGAGTT | CUCAGCAGUGGAUGACCAATT |
| | (SEQ ID NO: 424) | (SEQ ID NO: 425) |
| 227 | AUAACUGGCCACAUACUGCTT | GCAGUAUGUGGCCAGUUAUTT |
| | (SEQ ID NO: 426) | (SEQ ID NO: 427) |
| 231 | UACAUGGAGAGGAAGAAGGTT | CCUUCUUCCUCUCCAUGUATT |
| | (SEQ ID NO: 428) | (SEQ ID NO: 429) |
| 235 | UGUAAUAUGACUUGACAUCTT | GAUGUCAAGUCAUAUUACATT |
| | (SEQ ID NO: 430) | (SEQ ID NO: 431) |
| 223 | UCCAUGUGAGAAGAGUAAGTT | CUUACUCUUCUCACAUGGATT |
| | (SEQ ID NO: 432) | (SEQ ID NO: 433) |
| 228 | UGACGUUUGACAAAUGCUGTT | CAGCAUUUGUCAAACGUCATT |
| | (SEQ ID NO: 434) | (SEQ ID NO: 435) |
| 241 | UUGGUGACCCACUUACAAGTT | CUUGUAAGUGGGUCACCAATT |
| | (SEQ ID NO: 436) | (SEQ ID NO: 437) |
| 253 | UUCAUCGUGAGAAAUCUCCTT | GGAGAUUUCUCACGAUGAATT |
| | (SEQ ID NO: 438) | (SEQ ID NO: 439) |
| 269 | AAUGCUGUGGCAAAUAUCCTT | GGAUAUUUGCCACAGCAUUTT |
| | (SEQ ID NO: 440) | (SEQ ID NO: 441) |
| 246 | UGGUCAAUGCCAUCUUUGGTT | CCAAAGAUGGCAUUGACCATT |
| | (SEQ ID NO: 442) | (SEQ ID NO: 443) |
| 266 | AUCCCUACCCGAAAUGCACTT | GUGCAUUUCGGGUAGGGAUTT |
| | (SEQ ID NO: 444) | (SEQ ID NO: 445) |
| 262 | UAAGCAGAUCUAUAUUGGCTT | GCCAAUAUAGAUCUGCUUATT |
| | (SEQ ID NO: 446) | (SEQ ID NO: 447) |
| 277 | UCUCAUAUGUCUUUGACUGTT | CAGUCAAAGACAUAUGAGATT |
| | (SEQ ID NO: 448) | (SEQ ID NO: 449) |
| 280 | AUGAGGACCACUAGCAUGGTT | CCAUGCUAGUGGUCCUCAUTT |
| | (SEQ ID NO: 450) | (SEQ ID NO: 451) |
| 272 | UUGAUUGUCUGGUAAGAGCTT | GCUCUUACCAGACAAUCAATT |
| | (SEQ ID NO: 452) | (SEQ ID NO: 453) |
| 291 | ACAGAUUCUAGAAUGUUCCTT | GGAACAUUCUAGAAUCUGUTT |
| | (SEQ ID NO: 454) | (SEQ ID NO: 455) |
| 286 | UUUAACAACCUGUAUAACCTT | GGUUAUACAGGUUGUUAAATT |
| | (SEQ ID NO: 456) | (SEQ ID NO: 457) |
| 293 | UCCGGAAUACAAAUGAUGGTT | CCAUCAUUUGUAUUCCGGATT |
| | (SEQ ID NO: 458) | (SEQ ID NO: 459) |
| 290 | UUAUCUCUUUGCUUUCGAGTT | CUCGAAAGCAAAGAGAUAATT |
| | (SEQ ID NO: 460) | (SEQ ID NO: 461) |
| 276 | UACCACAUCAUGUUCUUAGTT | CUAAGAACAUGAUGUGGUATT |
| | (SEQ ID NO: 462) | (SEQ ID NO: 463) |
| 287 | AGCUGGGAGAUCCAAUAGGTT | CCUAUUGGAUCUCCCAGCUTT |
| | (SEQ ID NO: 464) | (SEQ ID NO: 465) |
| 315 | AUGUGGUGCAUCAUAGGUCTT | GACCUAUGAUGCACCACAUTT |
| | (SEQ ID NO: 466) | (SEQ ID NO: 467) |
| 310 | UCAUGGUGGAGAUCAUAUGTT | CAUAUGAUCUCCACCAUGATT |
| | (SEQ ID NO: 468) | (SEQ ID NO: 469) |
| 321 | UUUCGGAAGGCAGAUGAUCTT | GAUCAUCUGCCUUCCGAAATT |
| | (SEQ ID NO: 470) | (SEQ ID NO: 471) |
| 322 | UCAAGCAGGCCAAAGUAGGTT | CCUACUUUGGCCUGCUUGATT |
| | (SEQ ID NO: 472) | (SEQ ID NO: 473) |
| 311 | UCGGAAAUAGCGCACAUACTT | GUAUGUGCGCUAUUUCCGATT |
| | (SEQ ID NO: 474) | (SEQ ID NO: 475) |
| 304 | UAGCCGUUGCGGCAUACACTT | GUGUAUGCCGCAACGGCUATT |
| | (SEQ ID NO: 476) | (SEQ ID NO: 477) |
| 312 | UACAAGCGGAACUGUAUUGTT | CAAUACAGUUCCGCUUGUATT |
| | (SEQ ID NO: 478) | (SEQ ID NO: 479) |
| 305 | UUGGGUUGAUGAUUAGCUGTT | CAGCUAAUCAUCAACCCAATT |
| | (SEQ ID NO: 480) | (SEQ ID NO: 481) |
| 313 | UUCGGUAGUUGAGAUUCACTT | GUGAAUCUCAACUACCGAATT |
| | (SEQ ID NO: 482) | (SEQ ID NO: 483) |
| 323 | UACAGAUUGUUGUAGUAAGTT | CUUACUACAACAAUCUGUATT |
| | (SEQ ID NO: 484) | (SEQ ID NO: 485) |
| 333 | AGGCACAUCAUCAAUACCGTT | CGGUAUUGAUGAUGUGCCUTT |
| | (SEQ ID NO: 486) | (SEQ ID NO: 487) |
| 342 | AUGAUGUUGAAGACGUAGGTT | CCUACGUCUUCAACAUCAUTT |
| | (SEQ ID NO: 488) | (SEQ ID NO: 489) |
| 339 | AAUAUUCUUACCUAUCACCTT | GGUGAUAGGUAAGAAUAUUTT |
| | (SEQ ID NO: 490) | (SEQ ID NO: 491) |
| 327 | UCCAAUCUCAAUGUCAAACTT | GUUUGACAUUGAGAUUGGATT |
| | (SEQ ID NO: 492) | (SEQ ID NO: 493) |

In another embodiment, inhibiting expression of the RNAi target gene(s) in any of Tables 1, 2 and 3 comprises transfecting the host cell with an expression vector that comprises an inducible or non-inducible promoter operably linked to a nucleotide sequence that encodes a short hairpin RNA (shRNA) molecule capable of inhibiting expression of the RNAi target gene. The shRNA preferably targets an RNAi Target Sequence in Table 3.

In another aspect, the invention provides an siNA molecule for use in inhibiting expression of an RNAi target gene listed in Table 1, 2 or 3 above or an ortholog thereof. In an embodiment, the siNA molecule is an siRNA which comprises a sense strand and an antisense strand. The antisense strand comprises a first nucleotide sequence of at least 15 nucleotides that is complementary to at least 15 contiguous nucleotides of an RNAi target sequence selected from the group of sequences consisting of SEQ ID NOs:1-343 and the sense strand comprises a second nucleotide sequence of at least 15 nucleotides that is complementary to the first nucleotide sequence. In some embodiments, the RNAi target sequence is selected from the group of sequences consisting of SEQ ID NO:188, SEQ ID NO:149, SEQ ID NO:210, SEQ ID NO:228, SEQ ID NO:253, SEQ ID NO:269, SEQ ID NO:262, SEQ ID NO:272, SEQ ID NO:291, SEQ ID NO:339 and SEQ ID NO:327. In other embodiments, the antisense and sense strands of the siRNA consist of a pair of antisense and sense sequences selected from the group of siRNA sequences shown in Table 4. Preferably, the pair of antisense and sense sequences in an siRNA is the pair shown in Table 4 for an RNAi target sequence selected from the group consisting of SEQ ID NO:188, SEQ ID NO:149, SEQ ID NO:210, SEQ ID NO:228, SEQ ID NO:253, SEQ ID NO:269, SEQ ID NO:262, SEQ ID NO:272, SEQ ID NO:291, SEQ ID NO:339 and SEQ ID NO:327.

In another aspect, the invention provides an expression vector which comprises at least one expression cassette that is capable of expressing an shRNA in a mammalian host cell to inhibit expression of an RNAi Target Gene listed in Table 1, 2 or 3 above. In one embodiment, the expression cassette comprises an inducible or non-inducible promoter operably linked to a nucleotide sequence that encodes the shRNA molecule.

In yet another aspect, the invention provides a recombinant mammalian cell which is stably transfected with an expression cassette that is capable of expressing an shRNA that inhibits expression of an RNAi Target Gene or Target Sequence listed in Table 1, 2 or 3 above. In an embodiment, the recombinant mammalian cell further comprises at least one expression cassette that encodes an exogenous polypeptide.

### BRIEF DESCRIPTION OF THE FIGURES

FIG. 1 illustrates the structure of an expression vector suitable for use in expressing an exogenous polypeptide in the presence of an siNA molecule, with FIG. 1A showing the arrangement of various functional elements and restriction enzyme sites in the vector and FIGS. 1B and 1C showing the complete nucleotide sequence of the vector (SEQ ID NO:494).

FIG. 2 illustrates the structure of another expression vector suitable for use in expressing an exogenous polypeptide in the presence of an siNA molecule, with FIG. 2A showing the arrangement of various functional elements and restriction enzyme sites in the vector and FIGS. 2B and 2C showing the complete nucleotide sequence of the vector (SEQ ID NO:495).

FIG. 3 illustrates the structure of another expression vector suitable for use in expressing an exogenous polypeptide in the presence of an siNA molecule, with FIG. 3A showing the arrangement of various functional elements and restriction enzyme sites in the vector and FIGS. 3B and 3C showing the complete nucleotide sequence of the vector (SEQ ID NO:496).

FIG. 4 illustrates the effect of transient transfection of an siRNA targeting Wnk4 into recombinant CHO cells that express an exogenous mAb, with FIGS. 4A, 4B and 4C showing the intracellular expression level of mRNA for Wnk4, mAb light chain and mAb heavy chain, respectively, after 3 days of culturing the siRNA-transfected cell line (right bar) and a nontransfected control (left bar), and FIG. 4D showing the level of mAb in the supernatant after 5 days of culture of the siRNA-transfected cell line (right bar) and control cell line (left bar).

FIG. 5 illustrates the features of an exemplary vector useful in expressing an shRNA targeting Wnk4 and an exogenous mAb in stably transfected CHO cells.

FIG. 6 illustrates Wnk4 mRNA expression levels after 3 days of culturing the top 15 mAb producing clones of a CHO cell line that was stably transfected with the expression vector shown in FIG. 5 (shRNA knockdown bars) or a control expression vector that lacked the nucleotide sequence encoding the Wnk4 shRNA.

FIG. 7 illustrates the volumetric productivity of ten clones determined after 3 days of batch (passage) culture of a CHO cell line that was stably transfected with: the expression vector shown in FIG. 5 (shRNA, right bar) or a control expression vector that lacked the nucleotide sequence encoding the Wnk4 shRNA (Control, left bar).

FIG. 8 illustrates the volumetric productivity of ten clones determined at various time points during a 14 day fed-batch culture of a CHO cell line that was stably transfected with: the expression vector shown in FIG. 5 (shRNA, right bar) or a control expression vector that lacked the nucleotide sequence encoding the Wnk4 shRNA (Control, left bar).

FIG. 9 illustrates the specific productivity of ten clones determined after 14 days of fed batch culture of a CHO cell line that was stably transfected with: the expression vector shown in FIG. 5 (shRNA, right bar) or a control expression vector that lacked the nucleotide sequence encoding the Wnk4 shRNA (Control, left bar).

### DETAILED DESCRIPTION OF THE INVENTION

### I. General

The present invention is not to be limited in scope by the specific embodiments described herein. Indeed, various modifications of the invention in addition those described herein will become apparent to those skilled in the art from the foregoing description. Such modifications are intended to fall within the scope of the appended claims.

Patents, published patent applications, publications, product descriptions, and protocols are cited throughout this application, and the disclosure of such documents are incorporated herein by reference in their entirety for all purposes, and to the same extent as if each individual document was specifically and individually indicated to be incorporated by reference.

### II. Molecular Biology and Definitions

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

So that the invention may be more readily understood, certain technical and scientific terms are specifically defined below. Unless specifically defined elsewhere in this specification, all other technical and scientific terms use herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

Any numerical range of a parameter (e.g., concentration range, percentage range, nucleotide sequence length) is intended to include the endpoints and the value of any integer between the endpoints, and when appropriate, fractions thereof (such as one tenth and one hundredth of an integer), unless otherwise stated or otherwise evident from the context.

“Abasic” as used herein refers to its meaning as is generally accepted in the art. The term generally refers to sugar moieties lacking a nucleobase or having a hydrogen atom (H) or other non-nucleobase chemical groups in place of a nucleobase at the Γ position of the sugar moiety, see for example Adamic et al., U.S. Pat. No. 5,998,203. In one embodiment, an abasic moiety of the invention is a ribose, deoxyribose, or dideoxyribose sugar.

“About”, when used to modify a numerically defined parameter, e.g., the length of a siNA described herein, means that the parameter may vary by as much as 10% below or above the stated numerical value for that parameter, unless otherwise stated or evident from the context (e.g., where such number would exceed 100% of a possible value). For example, a siRNA comprising about 20 base pairs may comprise between 18 and 22 base pairs.

“Accession number” refers to an identification number for a transcript that is catalogued by the National Center for Biotechnology Information (NCBI), with more information about the transcript and the gene expressing the transcript available at www.ncbi.nlm.nih.gov.

“Acyclic nucleotide” refers to any nucleotide having an acyclic ribose sugar, for example where any of the ribose carbon/carbon or carbon/oxygen bonds are independently or in combination absent from the nucleotide.

“Alkyl” generally refers to saturated or unsaturated hydrocarbons, including straight-chain, branched-chain, alkenyl, alkynyl groups and cyclic groups, but excludes aromatic groups. Notwithstanding the foregoing, alkyl also refers to non-aromatic heterocyclic groups. Preferably, the alkyl group has 1 to 12 carbons. More preferably, it is a lower alkyl of from I to 7 carbons, more preferably 1 to 4 carbons. The alkyl group can be substituted or unsubstituted. When substituted, the substituted group(s) is preferably, hydroxyl, halogen, cyano, C1-C4 alkoxy, =0, ═S, N0₂, SH, NH₂, or NR₁R₂, where R₁ and R₂ independently are H or C1-C4 alkyl.

“Antisense region” and “Antisense strand” as used in reference to an siNA molecule means a nucleotide sequence of the siNA molecule having at least 80%, 85%, 90% or 95% complementarity to a target nucleic acid sequence. The antisense region of an siNA molecule may be referred to as the guide strand.

“Asymmetric hairpin” refers to a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

“Blunt end” as used in reference to a double-stranded siNA molecule means an end of the molecule with no overhanging nucleotides. For example, the two strands of a double-stranded siNA molecule having blunt ends align with each other with matched base-pairs without overhanging nucleotides at the termini. A double-stranded siNA molecule can comprise blunt ends at one or both of the termini located at the 5′-end of the antisense strand and the 5′-end of the sense strand.

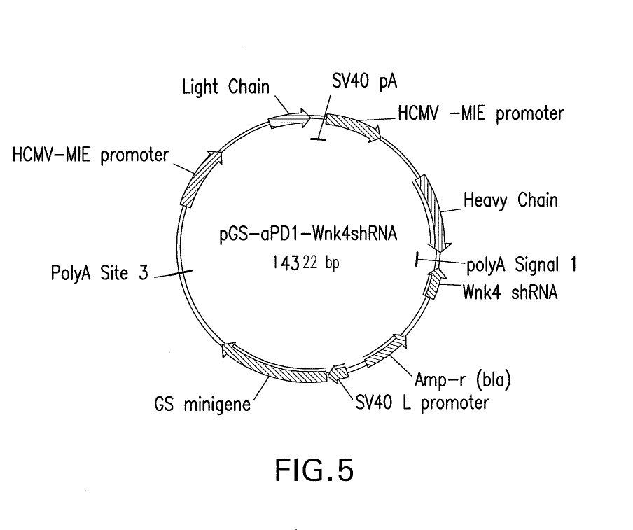

“Cap” or “Terminal cap” refers to a moiety, which can be a chemically modified nucleotide or non-nucleotide that can be incorporated at one or more termini of an siNA molecule. These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini of any nucleic acid molecule of the invention. A cap can be present at the 5′-end, 3-end and/or 5′ and 3′-ends of the sense strand of a nucleic acid molecule of the invention. Additionally, a cap can optionally be present at the 3′-end of the antisense strand of an siNA. In non-limiting examples, the 5′-cap includes, but is not limited to, LNA; glyceryl; inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; t zero-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3-phosphorothioate; phosphorodithioate; or bridging or non-bridging methylphosphonate moiety. Non-limiting examples of the 3′-cap include, but are not limited to, LNA; glyceryl; inverted deoxy abasic residue (moiety); 4′,5′-methylene nucleotide; 1-(beta-D-erythrofuranosyl) nucleotide; 4′-thio nucleotide; carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate; phosphorothioate and/or phosphorodithioate; bridging or non-bridging methylphosphonate; and 5′-mercapto moieties (for more details see Beaucage and Iyer, 1993, Tetrahedron 49, 1925; incorporated by reference herein). FIG. 5 shows some non-limiting examples of various caps.

“Coding sequence” is a nucleotide sequence that encodes a biological product of interest (e.g., an RNA, polypeptide, protein, or enzyme) and when expressed, results in production of the product. A coding sequence is “under the control of”, “functionally associated with” or “operably linked to” or “operably associated with” transcriptional or translational control sequences in a cell when the sequences direct RNA polymerase mediated transcription of the coding sequence into RNA, e.g., mRNA, which then may be trans-RNA spliced (if it contains introns) and, optionally, translated into a protein encoded by the coding sequence.

“Complementary” and “Complementarity” as applied to two nucleotide sequences refers to the ability of the two nucleotide sequences to form a duplex structure by hydrogen bonded base pairs (e.g., a duplex formed by an RNAi target sequence and a nucleotide sequence in an siNA or in a duplex region of an siRNA molecule). Perfect complementarity means that all the contiguous residues of one of the nucleotide sequences in the duplex will hydrogen bond with the same number of contiguous residues in the other nucleotide sequence. Partial complementarity can include 1, 2, 3, 4, 5 or more mismatches, non-base paired nucleotides, or non-nucleotide linkers, which can result in bulges, loops and/or overhangs, and can be represented by a percent (%) complementarity that is determined by the number of non-base paired nucleotides, e.g., 50%, 60%, 70%, 80%, 90%, etc., depending on the total number of nucleotides involved. For example, for a duplex formed between two sequences of 19 nucleotides, one mismatch has 94.7% complementarity, and four mismatches have 78.9% complementarity. In preferred embodiments of the various methods and compositions described herein, the complementarity in a duplex formed by two specified nucleotide sequences is at least about 80%, 85%, 90%, 95% or 100%.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified method or composition.

“Express” and “expression” mean allowing or causing the information in a gene or coding sequence, e.g., an RNA or DNA, to become manifest; for example, producing a protein by activating the cellular functions involved in transcription and translation of a corresponding gene. A DNA sequence can be expressed in or by a cell to form an “expression product” such as an RNA (e.g., mRNA) or a protein. The expression product itself may also be said to be “expressed” by the cell.

“Expression vector” or “expression construct” means a vehicle (e.g., a plasmid) by which a polynucleotide comprising regulatory sequences operably linked to a coding sequence can be introduced into a host cell where the coding sequence is expressed using the transcription and translation machinery of the host cell.

“Host cell” includes any cell of any organism that is manipulated by a human for the purpose of producing an expression product encoded by an expression vector introduced into the host cell. A “recombinant mammalian host cell” refers to a mammalian cell that comprises a heterologous expression vector, which may or may not be integrated into a host cell chromosome.

“Hybridization conditions” means the combination of temperature and composition of the hybridization solution that are used in a hybridization reaction between at least two oligonucleotides (see e.g., Sambrook et al, Molecular Cloning: A Laboratory Manual, 2nd Ed., Cold Spring Harbor Laboratory Press, Cold Spring Harbor, N.Y. (1989)). Preferred stringent hybridization conditions include overnight incubation at 42° C. in a solution comprising: 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

“Isolated” refers to the purification status of a biological molecule such as RNA, DNA, oligonucleotide, polynucleotide or protein, and in such context means the molecule is substantially free of other biological molecules such as nucleic acids, proteins, lipids, carbohydrates, or other material such as cellular debris and growth media. Generally, the term “isolated” is not intended to require a complete absence of other biological molecules or material or an absence of water, buffers, or salts, unless they are present in amounts that substantially interfere with the methods of the present invention.

“Nucleic acid” refers to a single- or double-stranded polymer of bases attached to a sugar phosphate backbone, and includes DNA and RNA molecules.

“Oligonucleotide” refers to a nucleic acid that is usually between 5 and 100 contiguous nucleotides in length, and most frequently between 10-50, 10-40, 10-30, 10-25, 10-20, 15-50, 15-40, 15-30, 15-25, 15-20, 20-50, 20-40, 20-30 or 20-25 contiguous nucleotides in length.

“Ortholog” or “Orthologous” means, with respect to a specific target RNAi sequence disclosed in Table 2, a sequence that is present in a different mammalian species than the Table 2 target sequence and is capable of hybridizing under high stringency conditions to the complement of the Table 2 target sequence.

“Polynucleotide” refers to a nucleic acid that is 13 or more contiguous nucleotides in length.

“Promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. Within the promoter sequence may be found a transcription initiation site (conveniently defined, for example, by mapping with nuclease S1), as well as protein binding domains (consensus sequences) responsible for the binding of RNA polymerase as well an enhancer element.

“Promoter activity” refers to a physical measurement of the strength of the promoter.

“RNA” as used herein refers to a molecule comprising at least one ribofuranoside moiety. The term can include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

“RNA interference” or “RNAi” refer to the biological process of inhibiting or down regulating gene expression in a cell, as is generally known in the art, and which is mediated by short interfering nucleic acid molecules, see for example Zamore and Haley, 2005, Science, 309, 1519-1524; Vaughn and Martienssen, 2005, Science, 309, 1525-1526; Zamore et al, 2000, Cell, 101, 25-33; Bass, 2001, Nature, 411, 428-429; Elbashir et al, 2001, Nature, 411, 494-498; and Kreutzer et al, PCT Publication No. WO 00/44895; Zernicka-Goetz et al, PCT Publication No. WO 01/36646; Fire, PCT Publication No. WO 99/32619; Plaetinck et al, PCT Publication No. WO00/01846; Mello and Fire, PCT Publication No. WO 01/29058; Deschamps-Depaillette, PCT Publication No. WO 99/07409; and Li et al, PCT Publication No. WO 00/44914; Allshire, 2002, Science, 297, 1818-1819; Volpe et al, 2002, Science, 297, 1833-1837; Jenuwein, 2002, Science, 297, 2215-2218; and Hall et al, 2002, Science, 297, 2232-2237; Hutvagner and Zamore, 2002, Science, 297, 2056-60; McManus et al, 2002, RNA, 8, 842-850; Reinhart et al, 2002, Gene & Dev., 16, 1616-1626; and Reinhart & Bartel, 2002, Science, 297, 1831). Additionally, the term RNAi is meant to be equivalent to other terms used to describe sequence specific RNA interference, such as post transcriptional gene silencing, translational inhibition, transcriptional inhibition, or epigenetics. For example, expression of an RNAi target gene or sequence described herein may be inhibited at either the post-transcriptional level or the pre-transcriptional level. In a non-limiting example, epigenetic modulation of gene expression by an siNA molecule can result from siNA mediated modification of chromatin structure or methylation patterns to alter gene expression (see, for example, Verdel et al, 2004, Science, 303, 672-676; Pal-Bhadra et al, 2004, Science, 303, 669-672; Allshire, 2002, Science, 297, 1818-1819; Volpe et al, 2002, Science, 297, 1833-1837; Jenuwein, 2002, Science, 297, 2215-2218; and Hall et al, 2002, Science, 297, 2232-2237). In another non-limiting example, modulation of gene expression by an siNA molecule can result from siNA mediated cleavage of RNA (either coding or non-coding RNA) via RISC, or via translational inhibition, as is known in the art, or modulation can result from transcriptional inhibition (see for example Janowski et al, 2005, Nature Chemical Biology, 1, 216-222).

“Sense region” and “Sense strand” as used in reference to a target gene means the region or strand of the gene that comprises a coding sequence, and as used in reference to a siNA means a region or strand that has sequence homology or identity with a target sequence. In one embodiment, the sense region or strand of a siNA molecule is also referred to as the passenger strand.

“Short interfering nucleic acid molecule” or “siNA molecule” refers to a single-stranded or double-stranded nucleic acid molecule that is capable of inhibiting the expression of an RNAi target gene or sequence disclosed herein when transfected into or expressed within a host mammalian cell. The inhibiting activity of a siNA molecule is achieved by mediating RNAi or gene silencing in a sequence-specific manner, including but not limited to Argonaute-mediated post-transcriptional cleavage of mRNA transcripts of the target gene. The siNA molecule comprises a nucleotide sequence of about 15 to about 30 nucleotides that is substantially complementary to a sequence in the target gene, which may be present in one or more of the coding region, the promoter region, the 3′ untranslated region and the 5′ untranslated region. siNA molecules useful in inhibiting the RNAi targets described herein include, but are not limited to, siRNA, short hairpin RNA (shRNA), double-stranded RNA (dsRNA), micro-RNA (miRNA), and circular RNA molecules. A single stranded siNA molecule may have one or more double-stranded regions and a double-stranded siNA molecule may have one or more single-stranded regions. The siNA can be a double-stranded nucleic acid molecule comprising self-complementary sense and antisense strands, wherein the antisense strand comprises a nucleotide sequence that is complementary to a nucleotide sequence in a target nucleic acid molecule or a portion thereof and the sense strand comprises a nucleotide sequence corresponding to the target nucleic acid sequence or a portion thereof. The siNA can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region comprises a nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to a nucleotide sequence in a target nucleic acid molecule or a portion thereof and the sense region comprises a nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single-stranded polynucleotide having a nucleotide sequence complementary to nucleotide sequence in a target nucleic acid molecule or a portion thereof (for example, where such siNA molecule does not require the presence within the siNA molecule of a nucleotide sequence corresponding to the target nucleic acid sequence or a portion thereof), wherein the single-stranded polynucleotide can further comprise a terminal phosphate group, such as a 5′-phosphate (see for example, Martinez et al, 2002, Cell, 110, 563-574 and Schwarz et al, 2002, Molecular Cell, 10, 537-568), or 5′,3′-diphosphate.

“Selectable marker” is a protein which allows the specific selection of cells which express this protein by the addition of a corresponding selecting agent to the culture medium.

“siRNA” as used herein means a short (15 to 30 nucleotides) dsRNA with a duplex region and 2 overhanging nucleotides at the phosphorylated 5′ ends and hydroxylated 3′ ends. Preferably the duplex region is 17 to 25 base pairs, 19 to 23 base pairs, or 19 base pairs.

“Transfecting”, “transfection” or “transfected” refers to introducing a siNA into a cell, which may be achieved by passive delivery, or by use of chemical or mechanical means that enhance uptake of the siNA by the cell. Transfection means include, but are not limited to, electroporation, particle bombardment, calcium phosphate delivery, DEAE-dextran delivery, lipid delivery, polymer delivery, molecular conjugate delivery (e.g., polylysine-DNA or -RNA conjugates, antibody-polypeptide conjugates, antibody-polymer conjugates, or peptide conjugates), microinjection, laser- or light-assisted microinjection, optoporation or photoporation with visible and/or nonvisible wavelengths of electromagnetic radiation. In one embodiment, passive delivery includes conjugating the siNA to a moiety that facilitates delivery to the cell, such as, e.g., cholesterol or a cholesterol derivative as described in U.S. Pat. No. 8,273,722.

“Universal base” as used herein generally refers to nucleotide base analogs that form base pairs with each of the natural DNA/RNA bases with little or no discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see, for example, Loakes, 2001, Nucleic Acids Research, 29, 2437-2447).

### III. Preferred Embodiments of the Invention

The present invention is directed to culturing a recombinant mammalian host cell under conditions in which expression of at least one endogenous gene is inhibited and thereby results in improved production of an exogenous polypeptide. The endogenous gene is any of the specific target genes listed in Table 1, 2 or 3 or an ortholog thereof. Expression of two or more of these RNAi targets may be inhibited at the same time or at different times during the cell culture.

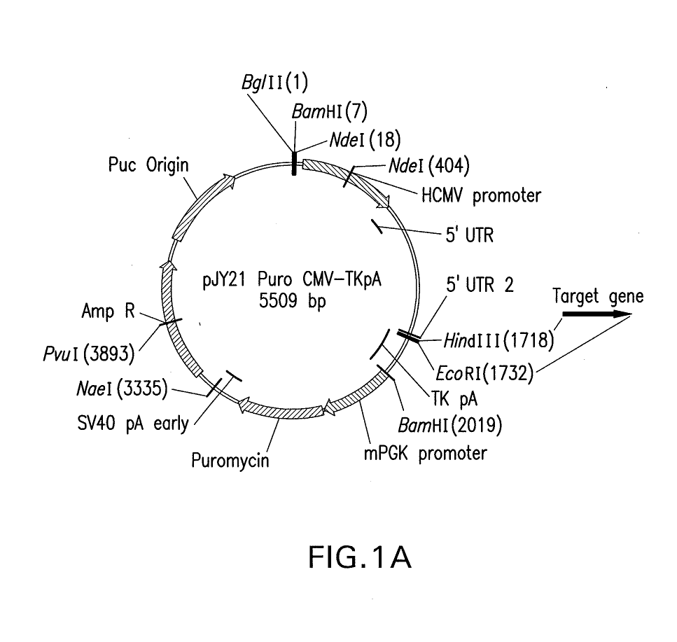

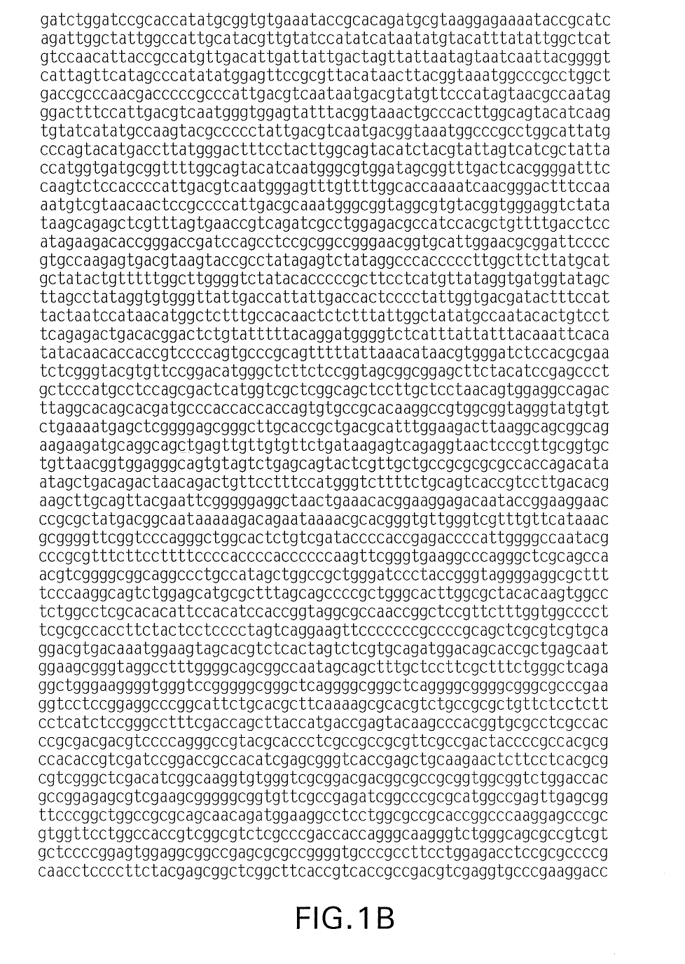

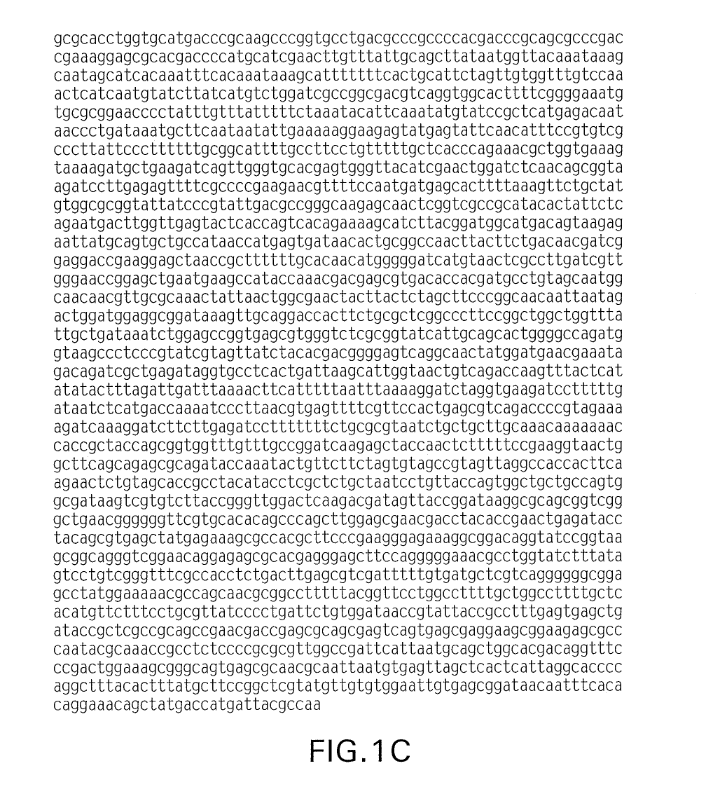

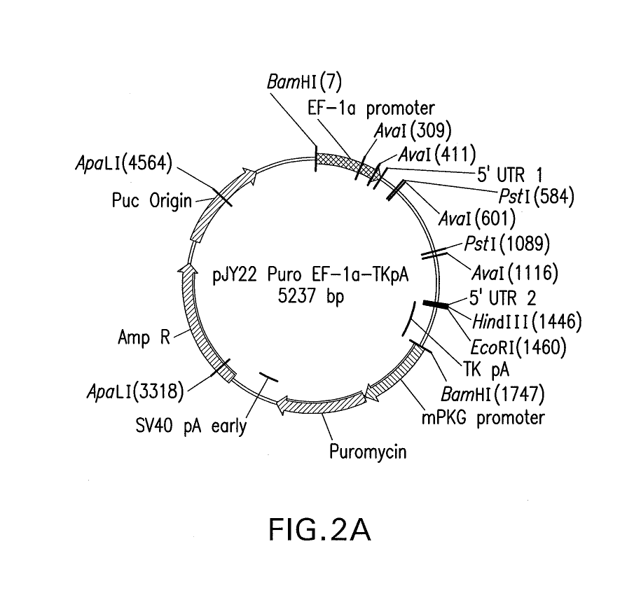

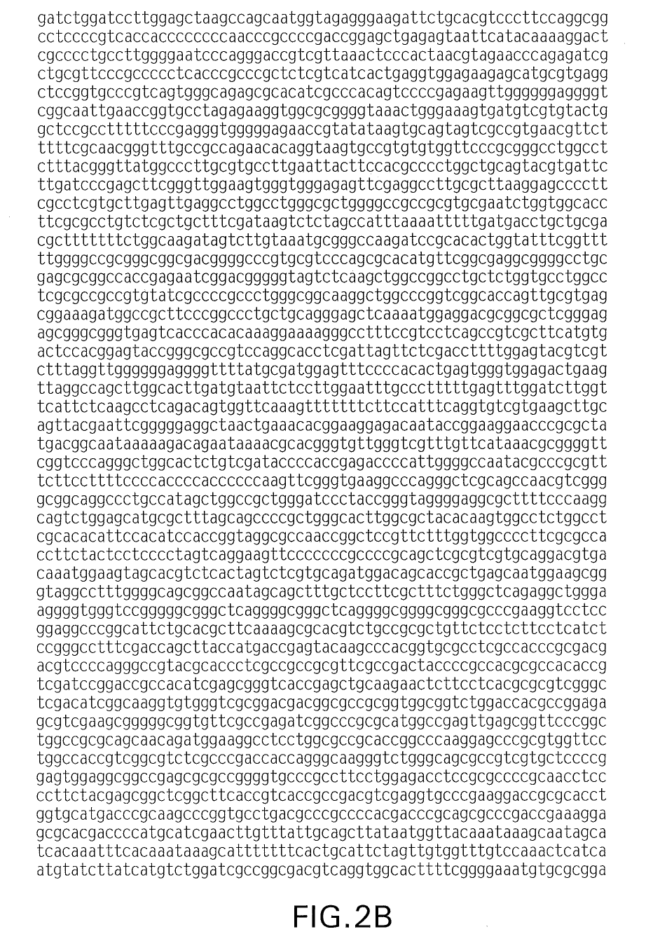

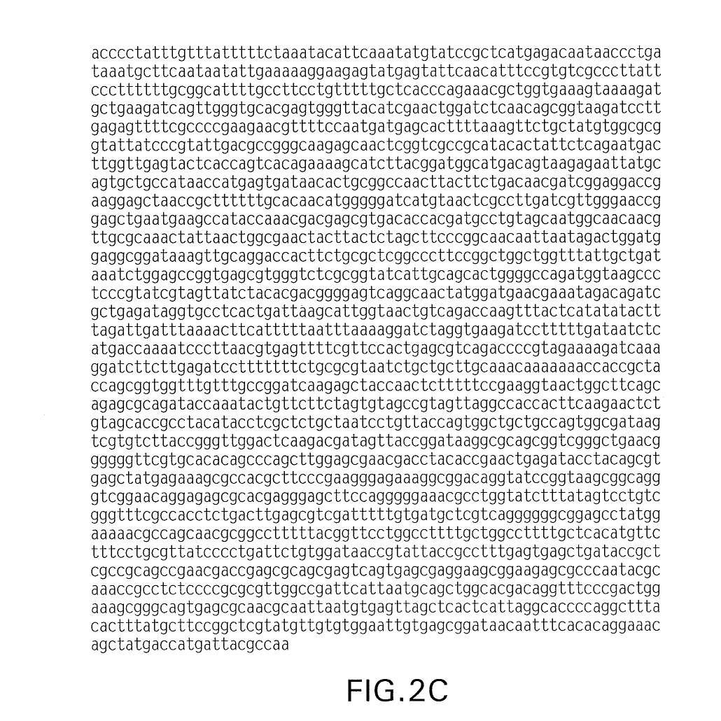

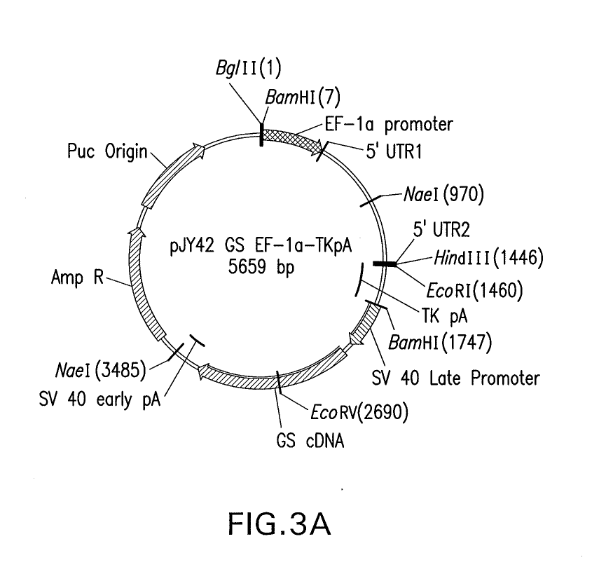

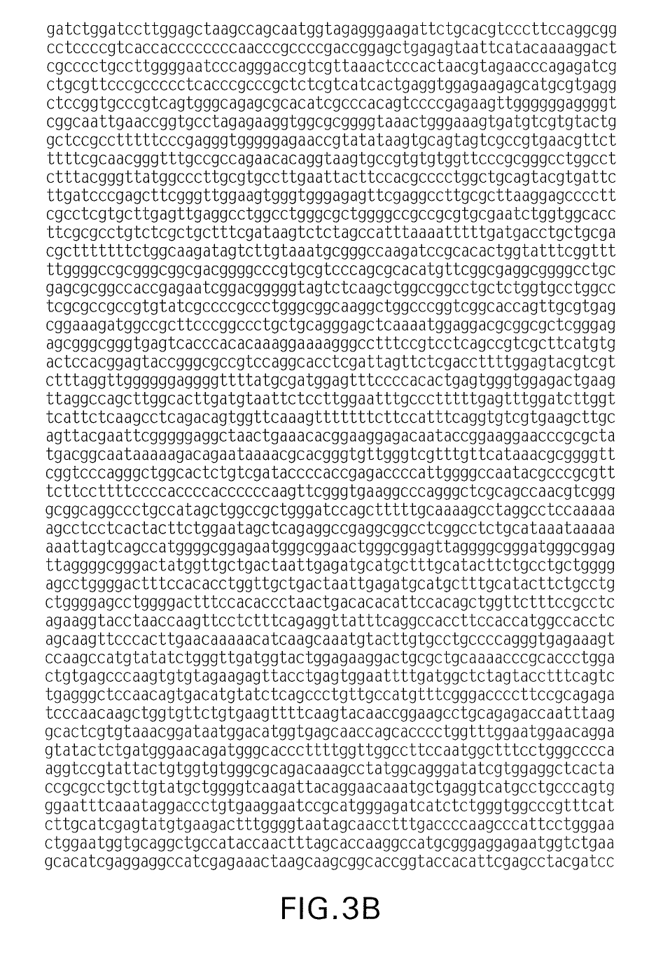

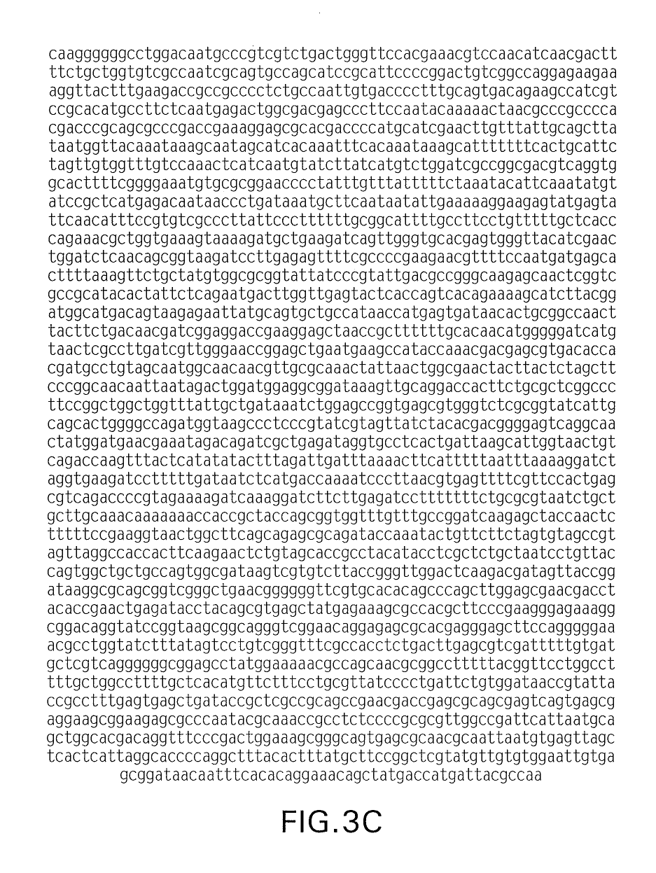

Exogenous polypeptides that may be produced using the methods of the invention include, but are not limited to, therapeutic polypeptides such as adhesion molecules, antibody light and/or heavy chains, cytokines, enzymes, lymphokines, and receptors. The exogenous polypeptide may be expressed in a mammalian host cell from an expression vector that has been transiently or stably transfected into the host cell. Examples of expression vectors suitable for exogenous polypeptide expression are illustrated in FIGS. 1-4 herein and further described in WO2014/100073.

Mammalian cells useful as host cells in the cell culture methods of the present invention include human cells, non-human primate cells and rodent cells. Suitable mammalian host cells include hamster cells such as BHK21, BHK TK⁻, CHO, CHO-K1, CHO-DUKX, CHO-DUKX B1 and CHO-DG44 cells or derivatives/descendants of these cell lines. Preferred host cells are CHO-DG44, CHO-DBX11, CHO-DUKX, CHO-K1 and BHK21 cells. Also suitable are myeloma cells from the mouse, preferably NS0 and Sp2/0-AG14 cells and human cell lines such asHEK293 or PER.C6, as well as derivatives/descendants of these mouse and human cell lines.

Cell culture conditions that inhibit expression of the target gene(s) include the presence within the host cell of a siNA molecule for each target gene. The siNA molecule(s) may be transfected into the host cell before the start of the culture or during the growth or production phase of the culture. In one embodiment, the host cell is transfected with the siNA molecule prior to culturing the host cell in a bioreactor. In another embodiment, the method comprises culturing the host cell for a first time period in a bioreactor, transfecting at least a portion of the cells in the bioreactor with the siNA, and culturing the transfected cells for a second time period in the bioreactor. Alternatively, the siNA molecule(s) may be expressed by the host cell from expression construct(s) stably integrated into the host cell genome.

Any amount of inhibition of target gene expression that results in at least a 25% increase in the level of the exogenous polypeptide produced in the presence of the siNA molecule(s) as compared to in the absence of the siNA molecule(s) under otherwise identical cell culture conditions is contemplated as being within the scope of the present invention. Thus, complete inhibition of target gene expression by the methods and siNA molecules of the invention may not be required. For example, depending on the function of the protein encoded by a target gene, or the degree of the negative impact of the endogenous protein on production of the exogenous polypeptide, inhibiting target gene expression by at least 10%, 20%, 30%, 40%, 60%, 70%, 80% or 90% may result in a 25% increase in yield of the exogenous polypeptide. In some specific embodiments, inhibition of one or more of the RNAi target genes produces an increased yield of the exogenous polypeptide of at least 30%, 40%, 50% or more as compared to the yield produced by the same host cell under the same cell culture conditions, but with no RNAi target inhibition.

The amount of target gene inhibition can be measured by a variety of methods, which can include measurement of transcript levels by Northern Blot Analysis, B-DNA techniques, quantitative PCR analysis, transcription-sensitive reporter constructs, expression profiling (e.g., DNA chips), and related technologies and assays. Alternatively, target gene inhibition can be measured by assessing the level of the protein encoded by target gene. This can be accomplished by performing a number of studies including Western Analysis, ELISA, measuring the levels of expression of a reporter protein, such as colorimetric or fluorescent properties (e.g., GFP), enzymatic activity (e.g., alkaline phosphatases), or other known analytical procedures.

The skilled artisan may readily design and manufacture siNA molecules suitable for inhibiting expression of the target genes described herein using techniques and processes well-known in the art, e.g., as described in WO2012/170284, WO2011/005793, U.S. Pat. No. 8,273,722, WO2005/097992, WO2008/036825, US2004/0266707, WO2004/090105, U.S. Pat. No. 5,889,136, Vermeulen A, et. al.; RNA 11:674-682 (2005). Particular aspects of various embodiments of the cell culture methods and siNA molecules of the invention are described below.

The siNA molecules can be provided in several forms. For example, an siNA molecule can be prepared from one or more chemically synthesized synthetic oligonucleotides, or it may take the form of a transcriptional cassette in a nucleic acid plasmid, i.e., expression vector. Two or more siNAs can be used to inhibit expression of a single RNAi target, or the expression of multiple RNAi targets may be inhibited by using a combination of one or more siNAs for each RNAi target.

The siNA can be single-stranded or double-stranded. Preferred embodiments of double-stranded siNA molecules comprise a sense and an antisense strand, where the antisense strand is complementary to at least a part of an mRNA formed in the expression of an RNAi target gene listed in any of Table 1, 2 or 3 above and the sense strand comprises a region that is complementary to the antisense strand. In specific embodiments, the antisense strand comprises at least 15 nucleotides of an antisense sequence selected from Table 4 and at least 15 nucleotides of a sense strand selected from Table 4. In some embodiments, the “at least 15 nucleotides” is 15 contiguous nucleotides.

A double stranded siNA molecule can be a double stranded RNA molecule, which can comprise two distinct and separate strands that can be symmetric or asymmetric and are complementary, i.e., two single-stranded RNA molecules, or can comprise one single-stranded molecule in which two complementary portions, e.g., a sense region and an antisense region, are base-paired, and are covalently linked by one or more single-stranded “hairpin” areas (i.e., loops) resulting in, for example, a single-stranded short-hairpin polynucleotide or a circular single-stranded polynucleotide. The linker can be a polynucleotide linker or a non-nucleotide linker. In some embodiments, the linker is a non-nucleotide linker. In some embodiments, a hairpin or circular siNA molecule of the invention contains one or more loop motifs, wherein at least one of the loop portions of the siNA molecule is biodegradable. For example, a single-stranded hairpin siNA molecule of the invention is designed such that degradation of the loop portion of the siNA molecule in vivo (i.e., in the host cell) can generate a double-stranded siNA molecule with 3′-terminal overhangs, such as 3′-terminal nucleotide overhangs comprising 1, 2, 3 or 4 nucleotides. Or alternatively, a circular siNA molecule of the invention is designed such that in vivo degradation of the loop portions of the siNA molecule can generate a double-stranded siNA molecule with 3′-terminal overhangs, such as 3′-terminal nucleotide overhangs comprising about 2 nucleotides.

In symmetric siNA molecules, each strand, the sense (passenger) strand and antisense (guide) strand, are independently about 15 to about 30 (e.g., about 15, 16, 17, 18, 19, 20, 21, 22, 23, 24, 25, 26, 27, 28, 29, or 30) nucleotides in length. Generally, each strand of the symmetric siNA molecules are about 19-24 (e.g., about 19, 20, 21, 22, 23 or 24) nucleotides in length.

In asymmetric siNA molecules, the antisense region or strand of the molecule is about 15 to about 30 (e.g., about 15, 16, 17, 18, 19, 20, 21, 22, 23, 24, 25, 26, 27, 28, 29, or 30) nucleotides in length, wherein the sense region is about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides in length. Generally, each strand of the asymmetric siNA molecules is about 19-24 (e.g., about 19, 20, 21, 22, 23 or 24) nucleotides in length.

In yet other embodiments, siNA molecules comprise single stranded hairpin siNA molecules, wherein the siNA molecules are about 25 to about 70 (e.g., about 25, 26, 27, 28, 29, 30, 31, 32, 33, 34, 35, 36, 40, 45, 50, 55, 60, 65, or 70) nucleotides in length.

In still other embodiments, siNA molecules comprise single-stranded circular siNA molecules, wherein the siNA molecules are about 38 to about 70 (e.g., about 38, 40, 45, 50, 55, 60, 65, or 70) nucleotides in length.

In still other embodiments, siNA molecules comprise single-stranded non-circular siNA molecules, wherein the siNA molecules are independently about 15 to about 30 (e.g., about 15, 16, 17, 18, 19, 20, 21, 22, 23, 24, 25, 26, 27, 28, 29, or 30) nucleotides in length.

In various symmetric embodiments, the siNA duplexes independently comprise about 15 to about 30 (e.g., about 15, 16, 17, 18, 19, 20, 21, 22, 23, 24, 25, 26, 27, 28, 29, or 30) base pairs. Generally, the duplex structure of the siNAs contains between 15 and 30, more generally between 18 and 25, yet more generally between 19 and 24, and most generally between 19 and 21 base pairs.

In yet other embodiments, where the duplex siNA molecules are asymmetric, the siNA molecules comprise about 3 to 25 (e.g., about 3, 4, 5, 6, 7, 8, 9, 10, 11, 12, 13, 14, 15, 16, 17, 18, 19, 20, 21, 22, 23, 24, or 25) base pairs. Generally, the duplex structure of the siNA contains between 15 and 25, more generally between 18 and 25, yet more generally between 19 and 24, and most generally between 19 and 21 base pairs.

In still other embodiments, where the siNA molecules are hairpin or circular structures, the siNA molecules comprise about 15 to about 30 (e.g., about 15, 16, 17, 18, 19, 20, 21, 22, 23, 24, 25, 26, 27, 28, 29, or 30) base pairs.

The sense strand and antisense strand, or the sense region and antisense region, of the siNA molecules can be complementary. Also, the antisense strand or antisense region can be complementary to a nucleotide sequence within an RNAi target gene represented by the Accession No. shown in any of Table 1, 2 or 3. The sense strand or sense region of the siNA can comprise a nucleotide sequence within an RNAi target gene represented by the Accession No. shown in any of Tables 1, 2 or 3. In certain embodiments, the antisense antisense strand or antisense region of an siNA molecule is complementary to the RNAi target sequence disclosed in any of Tables 1, 2 or 3.

In some embodiments, siNA molecules have perfect (i.e., 100%) complementarity between the sense strand or sense region and the antisense strand or antisense region of the siNA molecule. In other or the same embodiments, the antisense strand of the siNA molecules of the invention is perfectly complementary to an RNAi target sequence listed in any of Tables 1, 2 or 3.

In yet other embodiments, the siNA molecules have partial complementarity (i.e., less than 100% complementarity) between the sense strand or sense region and the antisense strand or antisense region of the siNA molecule or between the antisense strand or antisense region of the siNA molecule and the RNAi target. Thus, in some embodiments, the double-stranded siNA molecules have between about 15 to about 30 (e.g., about 15, 16, 17, 18, 19, 20, 21, 22, 23, 24, 25, 26, 27, 28, 29, or 30) nucleotides in one strand that are complementary to the nucleotides of the other strand. In other embodiments, the siNA molecules have between about 15 to about 30 (e.g., about 15, 16, 17, 18, 19, 20, 21, 22, 23, 24, 25, 26, 27, 28, 29, or 30) nucleotides in the sense region that are complementary to the nucleotides of the antisense region of the double-stranded nucleic acid molecule. In certain embodiments, the double-stranded siNA molecules of have between about 15 to about 30 (e.g., about 15, 16, 17, 18, 19, 20, 21, 22, 23, 24, 25, 26, 27, 28, 29, or 30) nucleotides in the antisense strand that are complementary to a nucleotide sequence within an RNAi target gene selected from any of Tables 1, 2 or 3.

In other embodiments, the siNA molecule can contain one or more nucleotide deletions, substitutions, mismatches and/or additions; provided, however, that the siNA molecule maintains its activity, for example, to mediate RNAi. In a non-limiting example, the deletion, substitution, mismatch and/or addition can result in a loop or bulge, or alternately a wobble or other alternative (non Watson-Crick) base pair. Thus, in some embodiments, for example, the double-stranded siNA molecules have 1 or more (e.g., 1, 2, 3, 4, 5, or 6) nucleotides, in one strand or region that are mismatches or non-base-paired with the other strand or region. In other embodiments, the double-stranded siNA molecules have 1 or more (e.g., 1, 2, 3, 4, 5, or 6) nucleotides in each strand or region that are mismatches or non-base-paired with the other strand or region. In one specific embodiment, the double-stranded siNA contains no more than 3 mismatches. If the antisense strand of the siNA contains mismatches to a target sequence, it is preferable that the area of mismatch not be located in the center of the region of complementarity.

In other specific embodiments, the siNA molecule can comprise at least one sequence selected from SEQ ID NOs: 1-343 (shown in Table 1) having one or more nucleotide deletions, substitutions, mismatches and/or additions to the selected sequence(s) provided, however, that the siNA molecule maintains its activity, for example, to mediate RNAi. In a non-limiting example, the deletion, substitution, mismatch and/or addition can result in a loop or bulge, or alternately a wobble or other alternative (non Watson-Crick) base pair.

The invention also includes double-stranded siNA molecules as otherwise described hereinabove in which the first strand and second strand are complementary to each other and wherein at least one strand is hybridisable to a polynucleotide sequence selected from SEQ ID NOs: 1-343 (shown in Table 1) under conditions of high stringency, and wherein any of the nucleotides is unmodified or chemically modified. In one specific embodiment, the first strand has about 15, 16, 17, 18, 19, 20 or 21 nucleotides that are complementary to the nucleotides of the other strand and at least one strand is hybridisable to a polynucleotide sequence selected from SEQ ID NOs: 1-67 and SEQ ID NOs: 440-506 (shown in Table 1b). In a more preferred embodiment, the first strand has about 15, 16, 17, 18, 19, 20 or 21 nucleotides that are complementary to the nucleotides of the other strand and at least one strand is hybridisable to SEQ ID NO: 7, SEQ ID NO: 446, SEQ ID NO: 11, SEQ ID NO: 450, SEQ ID NO: 12, SEQ ID NO: 451, SEQ ID NO: 13, SEQ ID NO: 452; SEQ ID NO: 38, SEQ ID NO: 477, SEQ ID NO: 39, SEQ ID NO: 478, SEQ ID NO: 40, SEQ ID NO: 479, SEQ ID NO: 41, SEQ ID NO: 480, SEQ ID NO: 59, SEQ ID NO: 498, SEQ ID NO: 63 or SEQ ID NO: 502; under conditions of high stringency, and wherein any of the nucleotides is unmodified or chemically modified.

In certain embodiments of the invention, the siNA molecules comprise overhangs of about 1 to about 4 (e.g., about 1, 2, 3 or 4) nucleotides. The nucleotides in the overhangs can be the same or different nucleotides. In some embodiments, the overhangs occur at the 3′-end at one or both strands of the double-stranded nucleic acid molecule. For example, a double-stranded siNA molecule can comprise a nucleotide or non-nucleotide overhang at the 3′-end of the antisense strand/region, the 3′-end of the sense strand/region, or both of the antisense strand/region and the sense strand/region of the double-stranded nucleic acid molecule.

In some embodiments, the nucleotides comprising the overhanging portion of an siNA molecule comprise sequences based on a sequence within an RNAi target gene in which the nucleotides comprising the overhanging portion of the antisense strand/region of the siNA molecule are complementary to nucleotides in the target sequence and/or the nucleotides comprising the overhanging portion of the sense strand/region of the siNA molecule can comprise nucleotides in the RNAi target sequence. Thus, in some embodiments, the overhang comprises a two nucleotide overhang that is complementary to a portion of a sense strand of the RNAi target gene. In other embodiments, however, the overhang comprises a two nucleotide overhang that is not complementary to the RNAi target. In certain embodiments, the overhang comprises a 3′-UU overhang that is not complementary to a portion of the RNAi target. In other embodiments, the overhang comprises a UU overhang at the 3′-end of the antisense strand and a TT overhang at the 3′-end of the sense strand.

In any of the embodiments of the siNA molecules described herein having 3′-terminal nucleotide overhangs, the overhangs are optionally chemically modified at one or more nucleic acid sugar, base, or backbone positions. Representative, but not limiting, examples of modified nucleotides in the overhanging portion of a double-stranded siNA molecule include: 2′-O-alkyl (e.g., 2′-O-methyl), 2′-deoxy, 2′-deoxy-2′-fluoro, 2′-deoxy-2′-fluoroarabino (FANA), 4′-thio, 2′-O-trifluoromethyl, 2′-O-ethyl-trifluoromethoxy, 2′-O-difluoromethoxy-ethoxy, universal base, acyclic, or 5-C-methyl nucleotides. In some specific embodiments, the overhang nucleotides are each independently, a 2′-O-alkyl nucleotide, a 2′-O-methyl nucleotide, a 2′-deoxy-2-fluoro nucleotide, or a 2′-deoxyribonucleotide. In some instances the overhanging nucleotides are linked by one or more phosphorothioate linkages.

In yet other embodiments of the invention, the siNA molecules comprise duplex nucleic acid molecules with blunt ends (i.e., without nucleotide overhangs), where both ends are blunt, or alternatively, where one of the ends is blunt. In some embodiments, the siNA molecules comprise one blunt end, for example wherein the 5′-end of the antisense strand and the 3′-end of the sense strand do not have any overhanging nucleotides, or wherein the 3′-end of the antisense strand and the 5′-end of the sense strand do not have any overhanging nucleotides. In other embodiments, the siNA molecules comprise two blunt ends, for example wherein the 3′-end of the antisense strand and the 5′-end of the sense strand, as well as the 5′-end of the antisense strand and 3′-end of the sense strand, do not have any overhanging nucleotides.

In any of the embodiments or aspects of the siNA molecules, the sense strand and/or the antisense strand can further have a cap, such as described herein or as known in the art, at the 3′-end, the 5′-end, or both of the 3′ and 5′-ends of the sense strand and/or antisense strand. In the case of a hairpin siNA molecule, the cap can be at either one or both of the terminal nucleotides of the polynucleotide. In some embodiments, the cap is at one or both ends of the sense strand of a double-stranded siNA molecule. In other embodiments, the cap is at the 3′-end of antisense (guide) strand. In preferred embodiments, a cap is at the 3′-end of the sense strand and at the 5′-end of the sense strand. Representative but non-limiting examples of such terminal caps include an inverted abasic nucleotide, an inverted deoxy abasic nucleotide, an inverted nucleotide moiety, a glyceryl modification, an alkyl or cycloalkyl group, a heterocycle, or any other cap as is generally known in the art.

Any of the embodiments of the siNA molecules can have a 5′ phosphate terminus. In some embodiments, the siNA molecules lack terminal phosphates.

The siNA molecules can comprise one or more chemical modifications. Modifications can be used to improve in vitro or in vivo characteristics such as stability, activity and toxicity. Non-limiting examples of chemical modifications that are suitable for use in the present invention, are disclosed in US 20040192626, US 20050266422, and US 20090176725, and in references cited therein, and include sugar, base, and phosphate modifications, non-nucleotide modifications, and or any combination thereof.

In various embodiments of the invention, the siNA molecules comprise modifications wherein any (e.g., one or more, or all) nucleotides present in the sense and/or antisense strand are modified nucleotides (e.g., wherein one nucleotide is modified, some nucleotides (i.e., a plurality or more than one) are modified, or all nucleotides are modified nucleotides). In some embodiments, the siNA molecules of the invention are partially modified (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, or 59 nucleotides are modified) with chemical modifications. In some embodiments, the siNA molecule comprises at least about 8, 10, 12, 14, 16, 18, 20, 22, 24, 26, 28, 30, 32, 34, 36, 38, 40, 42, 44, 46, 48, 50, 52, 54, 56, 58, or 60 nucleotides that are modified nucleotides. In other embodiments, the siNA molecules are completely modified (100% modified) with chemical modifications, i.e., the siNA molecule does not contain any ribonucleotides. In some embodiments, one or more of the nucleotides in the sense strand of the siNA molecules are modified. In the same or other embodiments, one or more of the nucleotides in the antisense strand of the siNA molecules are modified.

The chemical modification within a single siNA molecule can be the same or different. In some embodiments, at least one strand has at least one chemical modification. In other embodiments, each strand has at least one chemical modification, which can be the same or different, such as sugar, base, or backbone {i.e., internucleotide linkage) modifications. In other embodiments, the siNA molecule contains at least 2, 3, 4, 5 or more different chemical modifications.

In some embodiments of the invention, expression of the RNAi target is inhibited by siNA molecules, e.g., shRNA molecules, expressed from a transcription unit inserted into recombinant DNA or RNA vectors, such as DNA plasmids or viral vectors. siNA expressing viral vectors can be constructed based on, but not limited to, adeno-associated virus, retrovirus, adenovirus, or alphavirus. Transcription of the siNA molecule sequence can be driven from a promoter for eukaryotic RNA polymerase I (pol I), RNA polymerase II (pol II), or RNA polymerase III (pol III) (see, e.g., U.S. Pat. Nos. 5,902,880 and 6,146,886; Izant and Weintraub, 1985, Science, 229, 345; McGany and Lindquist, 1986, Proc. Natl Acad. Sci. 83, 399; Scanlon et al, 1991, Proc. Natl Acad. Sci., 88, 10591-5; Kashani-Sabet et al, 1992, Antisense Res. Dev., 2, 3-15; Dropulic et al, 1992, J Virol, 66, 1432-41; Weerasinghe et al, 1991, J Virol, 65, 5531-4; Ojwang et al, 1992, Proc. Natl Acad. Sci., 89, 10802-6; Chen et al, 1992, Nucleic Acids Res., 20, 4581-9; Sarver et al, 1990 Science, 247, 1222-1225; Thompson et al., 1995, Nucleic Acids Res., 23, 2259; Good et al., 1997, Gene Therapy, 4, 45). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters may also be used, providing that the prokaryotic RNA polymerase enzyme is expressed in the host cell (see Elroy-Stein and Moss, 1990, Proc. Natl Acad. Sci., 87, 6743-7; Gao and Huang 1993, Nucleic Acids Res., 21, 2867-72; Lieber et al, 1993, Methods Enzymol, 217, 47-66; Zhou et al, 1990, Mol. Cell. Biol, 10, 4529-37). Several investigators have demonstrated that nucleic acid molecules expressed from such promoters can function in mammalian cells (e.g., Yu et al, 1993, Proc. Natl. Acad. Sci., 90, 6340-4; L'Huillier et al, 1992, EMBO J., 11, 4411-8; Lisziewicz et al, 1993, Proc. Natl. Acad. Sci., 90, 8000-4; and Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (see, e.g., U.S. Pat. No. 5,624,803; Good et al, 1997, Gene Ther., 4, 45; and WO96/18736). The siNA transcription unit can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture et al, 1996, TIG, 12, 510).

In one embodiment, an shRNA transcription unit comprises a human U6 promoter operably linked to a nucleotide sequence encoding the shRNA and a transcription terminator. Exemplary nucleotide sequences for the U6 promoter and transcription terminator are set forth below.

U6 promoter (SEQ ID NO: 497):
CCCGAGTCCAACACCCGTGGGAATCCCATGGGCACCATGGCCCCTCGCTC
CAAAAATGCTTTCGCGTCGCGCAGACACTGCTCGGTAGTTTCGGGGATCA
GCGTTTGAGTAAGAGCCCGCGTCTGAACCCTCCGCGCCTCCCCGGCCCCA
GTGGAAAGACGCGCAGGCAAAACGCACCACGTGACGGAGCGTGACCGCGC
GCCGAGCGCGCGCCAAGGTCGGGCAGGAAGAGGGCCTATTTCCCATGATT
CCTTCATATTTGCATATACGATACAAGGCTGTTAGAGAGATAATTAGAAT
TAATTTGACTGTAAACACAAAGATATTAGTACAAAATACGTGACGTAGAA
AGTAATAATTTCTTGGGTAGTTTGCAGTTTTAAAATTATGTTTTAAAATG
GACTATCATATGCTTACCGTAACTTGAAAGTATTTCGATTTCTTGGCTTT
ATATATCTTGTGGAAAGGACGAAACACC
Transcription Terminator:
TTTTT

Vectors used to express the siNA molecules used in various embodiments of the invention can encode one or both strands of an siNA duplex, or a single self-complementary strand that self hybridizes into an siNA duplex. The nucleic acid sequences encoding the siNA molecules can be operably linked in a manner that allows expression of the siNA molecule in the recombinant mammalian host cell (see for example Paul et al, 2002, Nat. Biotechnol., 19, 505; Miyagishi and Taira, 2002, Nat. Biotechnol., 19, 497; and Lee et al, 2002, Nat. Biotechnol., 19, 500).

In some embodiments, vectors used to express one or more siNA molecules of the invention may also comprise one or more transcription units that encode an exogenous polypeptide. For example, any of the expression vectors shown in FIGS. 1-3 may be modified to express both the exogenous polypeptide and shRNA by inserting (1) an expression cassette that comprises a target gene that encodes the exogenous polypeptide and (2) an expression cassette that encodes an shRNA for an RNAi target.

In an embodiment, the expression vector is capable of producing an exogenous monoclonal antibody (mAb) and comprises first and second expression cassettes for the light chain and heavy chain of the mAb and an shRNA expression cassette located downstream of, and in the opposite orientation to, the second expression cassette. In one embodiment of such a mAb-expressing vector, the shRNA cassette expresses an shRNA that targets the Wnk4 gene (CHO accession number XM_003504666).

In an embodiment of the invention, the nucleotide sequence encoding a Wnk4 shRNA comprises:

(SEQ ID NO: 498)
GATACCTCAAGTTTGACATTGAGATTGGAGTCTAGAGTCCAATCTCAATG
TCAAACTTGAGGTATC.

In another embodiment of the invention, an shRNA expression cassette for targeting Wnk4 comprises:

(SEQ ID NO: 499)
CCCGAGTCCAACACCCGTGGGAATCCCATGGGCACCATGGCCCCTCGCTC
CAAAAATGCTTTCGCGTCGCGCAGACACTGCTCGGTAGTTTCGGGGATCA
GCGTTTGAGTAAGAGCCCGCGTCTGAACCCTCCGCGCCTCCCCGGCCCCA
GTGGAAAGACGCGCAGGCAAAACGCACCACGTGACGGAGCGTGACCGCGC
GCCGAGCGCGCGCCAAGGTCGGGCAGGAAGAGGGCCTATTTCCCATGATT
CCTTCATATTTGCATATACGATACAAGGCTGTTAGAGAGATAATTAGAAT
TAATTTGACTGTAAACACAAAGATATTAGTACAAAATACGTGACGTAGAA
AGTAATAATTTCTTGGGTAGTTTGCAGTTTTAAAATTATGTTTTAAAATG
GACTATCATATGCTTACCGTAACTTGAAAGTATTTCGATTTCTTGGCTTT
ATATATCTTGTGGAAAGGACGAAACACCGATACCTCAAGTTTGACATTGA
GATTGGAGTCTAGAGTCCAATCTCAATGTCAAACTTGAGGTATCTTTTT.

### Examples

These examples are intended to further clarify the present invention and not to limit the invention. Any composition or method, in whole or in part, set forth in the examples form a part of the present invention.

### Example 1: Generation of a CHO siRNA Library for RNAi Targets

The inventors herein generated a CHO siRNA screening library by comparing publicly available CHO or hamster genomic sequences with each target sequence in a proprietary siRNA library (owned by Merck and Co., Inc., USA), which contains about 20,000 siRNAs for about 6000 mouse genes and about 6000 rat genes, and identified 2,952 siRNA which are compatible with the hamster genomic sequences. Each siRNA of the CHO screening library comprises an RNAi target sequence of 19 nucleotides, with at least the middle 17 nucleotides having 100% sequence identity to a sequence within the hamster genomic sequence.

### Example 2: Screening of a CHO siRNA Library for RNAi Targets

To assess the impact of the siRNA in the CHO siRNA library generated in Example 1 on exogenous polypeptide expression, five different clones of established recombinant CHOK1 cell lines expressing three different monoclonal antibodies (mAbs) were used in the transfection and expression experiments described below. In brief, these cell lines comprise a dual-cassette expression vector, in which each of the heavy chain and light chain coding sequences of a mAb are operably linked to a cytomegalovirus (CMV) or elongation factor 1 alpha (EF-1a) promoter for expression of the mAb heavy chain or light chain. The expression vectors also had a puromycin or a glutamine synthetase selectable marker.

The recombinant CHOK1 cells were seeded in 96-well plates containing DMEM medium supplemented with 10% fetal bovine serum (FBS). Following the manufacturer's instruction, 100 pmol of an siRNA from the CHO siRNA library was mixed with Lipofectamine® RNAiMax (Invitrogen) and then transfected into CHO cells. Cells that were mock-transfected (no siRNA) with the Lipofectamine® RNAiMax were used as the baseline control.

Three to five days post-transfection, the supernatants of the transfected and mock-transfected cultures were collected and the mAb expression levels were measured using a modified microfluidic ELISA. Briefly, Gyros Bioaffy™ CD is coated with goat anti-human IgG (Jackson ImmunoResearch) as the capture reagent. The samples (culture supernatants) are then added followed by Alexa Fluor® 647-labeled goat anti-human IgG (Jackson ImmunoResearch). Based on the fluorescent signal detected by the machine, the antibody expression level is determined.

The antibody expression level determined for each siRNA transfection was then compared to the mock-transfected control. Of the CHO siRNA library of 2,952 siRNAs, 343 siRNAs were found to be able to improve the productivity by at least 25% as shown in Table 5A below.

TABLE 5A
Increased mAb production by CHO siRNA
molecules

| RNAi | SEQ ID | Relative Production |
|---|---|---|
| Target Sequence | NO. | Level (%) |
| CCTGCAAGCTCCGCTTCTA | 1 | 132 |
| CTTTGAAGACTTGAACAGT | 2 | 163 |
| CCAAGATTGCCAAGCGCAT | 3 | 135 |
| CATACCATTGGATGAGGTA | 4 | 137 |
| CTCTGCATCTTCATCCGGA | 5 | 140 |
| CCTACAAGCGCTTGTAGGA | 6 | 126 |
| CTCAACTTCTGTGCCAATA | 7 | 133 |
| GAAGCCCATTCCTCGAATT | 8 | 162 |
| GAGCTAAACTCCACCAAGA | 9 | 150 |
| CAGATACCACTTTCATCGA | 10 | 163 |
| CAAGTATGGCAGTATCATT | 11 | 154 |
| CAGTATGTCTCCAACGACA | 12 | 126 |
| GCGATATTATGAGCAACCT | 13 | 126 |
| CTACACATCTTCTGGCAGA | 14 | 186 |
| GCATGGAGGCCTTCGGCAT | 15 | 141 |
| GACATTGATTGCAATCCAT | 16 | 167 |
| CTCATACCTTCTTTATTAT | 17 | 126 |
| GAATGTATCTTCTGAAGGT | 18 | 126 |
| CATTCAGGCACGGATGGAA | 19 | 133 |
| GAACTAGAGCGGTTTGTAA | 20 | 193 |
| GTAATTGAGCTGTCAGTTT | 21 | 131 |
| CCATTTGTGCTGCAATAAT | 22 | 129 |
| GGACATATGTATTCGGGAA | 23 | 146 |
| CATAGTAGAGTATGATGAT | 24 | 128 |
| GAAACTTTACCCTTCTGAA | 25 | 151 |
| CTCTAAGGGAGCTCTGTTT | 26 | 132 |
| GTGATACCAGTGCAACTGA | 27 | 169 |
| GACATGCGCTGGCTACGAA | 28 | 150 |
| GGACTGGGTGCCTTACATC | 29 | 159 |
| CACAGTTTGGAATTACAGA | 30 | 292 |
| CTTGCCTGGTGACCGGGAA | 31 | 130 |
| GCATTATGCGGACATTCAT | 32 | 148 |
| CCTAGAAGATGAGCACCAA | 33 | 177 |
| CCAGAAATTCCATAAGCCT | 34 | 201 |
| CATTGACCAATGTTCGAGT | 35 | 170 |
| GAAACCAGGTCCACAGGAT | 36 | 132 |
| GCAGTTACGACTTGGGCAA | 37 | 130 |
| CACTGTATGACCTTCTGGT | 38 | 159 |
| CATTCCATTGTTGTTGATT | 39 | 133 |
| CATTCATCAGCAAGGTAAA | 40 | 140 |
| CAGAGTATGACGCCATGTT | 41 | 145 |
| GGATCAACAGGGTGTTGTT | 42 | 146 |
| CAATTACGTTTATAACACT | 43 | 125 |
| CAGTTTAACTTCCAATCCA | 44 | 246 |
| GTTTCGAAGTGCCTGTTTA | 45 | 164 |
| GACTTATATCCAGGACAAA | 46 | 144 |
| GCCAATAAATGACGTATTA | 47 | 142 |
| CTCATGGTTCTCTTGAACT | 48 | 126 |
| CTAACCACAGGTTCCGGAA | 49 | 142 |
| CCTTATGGTGTCTTGCAAT | 50 | 153 |
| CTTCCTTTAACAATCAGAT | 51 | 261 |
| CTTGCAACCAGTTTGGCCA | 52 | 141 |
| GCATGATTGTGGCCGAGGT | 53 | 141 |
| CAGATAACCACCACGATGT | 54 | 145 |
| GAAGGTACCTCCTGGTACA | 55 | 198 |
| CACATTTCTGTTAAAGGTT | 56 | 144 |
| GAGATGATCAGTTGGTACT | 57 | 126 |
| CAGGAATCCTGGAGAGTAA | 58 | 184 |
| GAATCTAGAGGATCTGGTT | 59 | 138 |
| GAACAAGGTTGTCATGGAT | 60 | 171 |
| CAGTATTGATGCAGTTTAT | 61 | 156 |
| GCTACAAGACCCTGCCTTT | 62 | 149 |
| CATACTCTATATCCATCTA | 63 | 160 |
| CGGATAAGAACGAGAGGTT | 64 | 140 |
| CGGATTTCAACTATTTCTT | 65 | 131 |
| CAACCAAGAAGAGGGCGAA | 66 | 138 |
| CTTACAACTGGATTCAAGA | 67 | 129 |
| GCAAGTATTTAGCATGAAT | 68 | 134 |
| GCCGTAAGGTGTACGGCAT | 69 | 126 |
| GTGCCTACAATAGCTGCTA | 70 | 166 |
| GAAATCAATGCCTGAGGCA | 71 | 145 |
| CTCATAAGAAGAGTTTCTT | 72 | 135 |
| GCACTTTGACCTGAATCTA | 73 | 128 |
| GGTTTATCCAGGAAGTATA | 74 | 235 |
| GTTATCATGTGGGAAGTGT | 75 | 166 |
| GGAGTTTATACCAAACAGA | 76 | 234 |
| CAAGTTCTGTGACGAGTGT | 77 | 251 |
| GAAGTTTGAAGGCTGTTAT | 78 | 138 |
| AGGATATTCACTTTCATAT | 79 | 187 |
| GACATCACCTCTGTTCTTC | 80 | 165 |
| GATGTCAACAGAGCCATCA | 81 | 158 |
| CTGTTGCCTGCTTACATTT | 82 | 154 |
| GCTACAAGCAGTCCTTGAA | 83 | 125 |
| GGATGATGAAGGTGTTCTT | 84 | 183 |
| CAATAAAGAAGATATAGTT | 85 | 146 |
| CTGTGTTGCCAATAGGGAA | 86 | 128 |
| GAATGTACCTGTCATGAAT | 87 | 146 |
| CCTCTATACCTCCATCTTT | 88 | 144 |
| GATTGCATGGCTGTTCAAT | 89 | 148 |
| GAATCTCGAAGCTTATATA | 90 | 153 |
| GTCAAGACCTGGTATGAGT | 91 | 128 |
| CAGATCATTGTTCATGCTT | 92 | 175 |
| GAGATGTGGCCCTTCTTCA | 93 | 176 |
| GCTCAAAGTGTGTAAGTTT | 94 | 126 |
| CCAAATATGGCTTGTACTT | 95 | 135 |
| CCTGGAATGTCTTCATTAT | 96 | 200 |
| GACATTCAGAGCCGAAGTA | 97 | 137 |
| GTGTTTGTGGAACTGCTTA | 98 | 324 |
| CTCAACATGTCTCGGTGTT | 99 | 137 |
| CTTGGTACTTGCTACGTGT | 100 | 146 |
| CAGCTAGCATCGGGACAGA | 101 | 128 |
| CATGTTCTATTGGATGTAT | 102 | 132 |
| GCACTGTTCCGCAAGGGCA | 103 | 153 |
| GGCAGTTCGACTATGCGTT | 104 | 153 |
| CCAAGTTTCATCTTATGCA | 105 | 137 |
| GAACTTTAAGGATATTACA | 106 | 137 |
| GAAGCATTATCCAGATCTA | 107 | 181 |
| CTGCTCTTCTGCAGGTGGA | 108 | 156 |
| GAAATGAGTCCACAGATGA | 109 | 136 |
| GGAGTATCATCTTGGGCTA | 110 | 159 |
| CGATTTGGATAGCCAACAA | 111 | 146 |
| CCACTTACTTCATGACCTA | 112 | 127 |
| CACTAATTCAGTTCCAGTA | 113 | 129 |
| GACAGTACCCAGCGACTTT | 114 | 227 |
| CACAGTGCTACTTCGAGTA | 115 | 125 |
| CAAGGAATTATCATTCAGA | 116 | 141 |
| GTGAGTGTGTGATCTGCCT | 117 | 153 |
| GCAACAGTGGCAGTGGGAA | 118 | 141 |
| GAATTACAAGGGCTACTTT | 119 | 157 |
| GCCTTCAAGGCGGAGATCA | 120 | 154 |
| CTACCTAGCTGACGAGATT | 121 | 153 |
| GGAATTCATGTCTAAGTCT | 122 | 150 |
| GAAATGCCTTTCAATGTCA | 123 | 160 |
| CTCTATGGGTTCCTGGTTA | 124 | 150 |
| CAGATTACTCCTGGATGAA | 125 | 127 |
| GCATTCCATTGTTGTTGAT | 126 | 142 |
| GTTATGGTCCTCATTCTTA | 127 | 132 |
| CCACAATTGTTGATGTGAA | 128 | 134 |
| GATGAAGATTGAGGAGTAT | 129 | 146 |
| GACAAATTGGATATTATTT | 130 | 136 |
| GAATTGAGCCACAGAGTAA | 131 | 126 |
| GAAACTGAAGGCTTTCTTA | 132 | 139 |
| GAACTACACATCTAAGTTT | 133 | 144 |
| CACAGTTCATCTTCGACCA | 134 | 139 |
| CAAAGAAGTTTGCAGATTT | 135 | 191 |
| CGAATGAAGGATTTCACCA | 136 | 131 |
| CCTTTGAGCTGGGCGAGAT | 137 | 216 |
| CTTTACCTGGAATATTACA | 138 | 135 |
| GTTCTTGTGACTCATTAGT | 139 | 133 |
| CACAGAACCTCTTGTTGGA | 140 | 132 |
| GTTAGAAATGGCTTCCGGA | 141 | 133 |
| GTCGTTATGGAACTCCTGA | 142 | 140 |
| GGATCTTTGGCTGGGCAGA | 143 | 160 |
| CTTTGACAACTGTTTACGA | 144 | 135 |
| GGAGTTAATCTCTTCGACA | 145 | 160 |
| CCATAGTCCTCTCCAAATT | 146 | 141 |
| GAATGACCCTGTCACTTTA | 147 | 127 |
| GGTACTTTCAGTTTCCAGA | 148 | 138 |
| CCGATTCTTGTATCATGGA | 149 | 136 |
| GCAAGTTGGTCAGACAGGA | 150 | 130 |
| GACGTATGGCTCAAATGGT | 151 | 130 |
| CACTACTTCATCGAGGTCA | 152 | 145 |
| CTAACATGCTGAATAGGTA | 153 | 143 |
| CAGATCTGTTTGAACGGAA | 154 | 168 |
| CCAAGTGTTCTTCAGGCAA | 155 | 136 |
| GTCATTAGAGTTGCCCAGA | 156 | 155 |
| CTCTAGAGTGCCAATCATA | 157 | 139 |
| CTCATTTAAGGTACCCTTT | 158 | 161 |
| CACTTGGATTGGCCTAACT | 159 | 199 |
| CACATCAAGTTCACCCAGA | 160 | 151 |
| CATGGATTCTTCACTCGTT | 161 | 168 |
| GTCATTGGGCTGCTCTATA | 162 | 177 |
| GCAATGTGTTCTTATGGAA | 163 | 160 |
| GAATTATGGGACGATTATT | 164 | 222 |
| GGAAGTATTTCACATGTGA | 165 | 198 |
| GAGAGTCCTGCCGGTGGTA | 166 | 133 |
| CAATGACTGCTCTTGTGCA | 167 | 139 |
| CATGCTATGAAGTACTCCA | 168 | 134 |
| CTATGATCCTGCCAAACGA | 169 | 238 |
| CCCTTTATCATTGAGTGGA | 170 | 128 |
| CACAGAAACCTTCCAGGTA | 171 | 159 |
| GCTGTTATGGATTAATTGT | 172 | 209 |
| GAGGAGTTTCCATCACGAA | 173 | 129 |
| GAGAAACGTCCTTTCATGT | 174 | 138 |
| CACACTTCTTCAAGGTGCT | 175 | 220 |
| GCTAGAAGATGGGACGGAG | 176 | 128 |
| CAAGAAGGATTTGACCGTA | 177 | 275 |
| CAGATCATCTTTGCAGTGA | 178 | 223 |
| CTCATTCATTTCCCAGTGT | 179 | 146 |
| CATCCAAGTGGATTGCTTT | 180 | 126 |
| CTACACTGGCCGTTTCTTA | 181 | 197 |
| CTATCCAGATGGTCGCCAA | 182 | 131 |
| CTGCTATTTCCATCCATGA | 183 | 174 |
| CCTATACCTTCAATTGCCA | 184 | 161 |
| CGATTTGCCACCAAAGCTT | 185 | 147 |
| CATCGAGCCTGACATCAAA | 186 | 155 |
| CCAACTATGGCATCGTCAC | 187 | 128 |
| GAGATCTACCGATACACAT | 188 | 125 |
| GTTCGAAGGCTGGTGGGAA | 189 | 174 |
| GCATTGACCGCTGTGTCAA | 190 | 143 |
| CCAACAAGGAGGAACTCAT | 191 | 139 |
| CAGAATTACTTAATAGAGA | 192 | 140 |
| CTATGTGTGTGATCTTGAA | 193 | 140 |
| GAAAGTTCCTGCCAGCAGT | 194 | 150 |
| CTACCAAACTTCGAGGATA | 195 | 173 |
| GAGCTGACATGCTCATGGT | 196 | 210 |
| CCTTCAAGAGGGCCACCTA | 197 | 182 |
| GTATTGATAAGCAACGTGA | 198 | 131 |
| CTTATGAGCTGGTGATCGA | 199 | 130 |
| GCAATTGTAACTTACCCTT | 200 | 134 |
| GCCATTATTCAGTGATGTT | 201 | 162 |
| CGATTCAACATTAACGATA | 202 | 130 |
| GTCGAATTGAACATTTATA | 203 | 138 |
| GATACCTTCTGGGTTCAGA | 204 | 134 |
| CTCGTTTGCCTTAATTCTT | 205 | 140 |
| GAGAGAAACTTGATGGGAA | 206 | 232 |
| CTAACAACCGGCTCCTCAA | 207 | 157 |
| GTCTCTAACACCATCGGAA | 208 | 189 |
| GAGATGATGATTGGATTCA | 209 | 149 |
| CATTGAGGACCTTCGTGTA | 210 | 150 |
| GAAGGATTGTGGTTATGGA | 211 | 141 |
| GGAGTCTGGTTTAACCTCA | 212 | 136 |
| GCTACATGACCGATGGACT | 213 | 132 |
| GCAAGTTTGTGCCCTCAGA | 214 | 162 |
| CTGACAAAGAGCTTCCACT | 215 | 142 |
| CCCATAACGGAGAATCTGT | 216 | 176 |
| CCAATGATGGCCATTCAGT | 217 | 153 |
| CCTTCAACATTGACCGAAA | 218 | 136 |
| CATTCGAAACCACAAATCT | 219 | 152 |
| GAAATACTGTCTCCCTTTA | 220 | 126 |
| GAAATACTACCATCTTACT | 221 | 134 |
| GGTGTAAATTAAGTAGCAA | 222 | 139 |
| CTTACTCTTCTCACATGGA | 223 | 135 |
| CTATTGGATTTACCATGTA | 224 | 159 |
| GAGATGGTGTACTGCTACA | 225 | 142 |
| GTACTTCCGAGAACTCTTT | 226 | 183 |
| GCAGTATGTGGCCAGTTAT | 227 | 127 |
| CAGCATTTGTCAAACGTCA | 228 | 169 |
| GTATGAAACGGCTAGAATT | 229 | 127 |
| CTCAGCAGTGGATGACCAA | 230 | 168 |
| CCTTCTTCCTCTCCATGTA | 231 | 145 |
| CTCTTGGACTACCTTATGA | 232 | 127 |
| CTTTGGACCTCGACTCATT | 233 | 148 |
| CTCAGAATTTGCTGACCCT | 234 | 125 |
| GATGTCAAGTCATATTACA | 235 | 137 |
| GATAGTTTATTGGGTGCTT | 236 | 127 |
| CAAGAAGAAGCGTTTCTGA | 237 | 128 |
| GCTTTCAACTGTATGGTCT | 238 | 189 |
| GATACTGGGTGTTTAAGGA | 239 | 128 |
| GGAATTGTACCTCGATCTT | 240 | 154 |
| CTTGTAAGTGGGTCACCAA | 241 | 133 |
| GAAATATATAGCTTTGGAA | 242 | 132 |
| CACTGTACCTCTAATTACA | 243 | 128 |
| CCTATTGGAATCTGGGCTA | 244 | 137 |
| TGTTTATGACCCTGGCTTA | 245 | 128 |
| CCAAAGATGGCATTGACCA | 246 | 135 |
| GTTTGCATCCGGCACCTGA | 247 | 144 |
| GTCTATGTCTTCATTCAGA | 248 | 152 |
| CATGGATTTGGATGTAGTA | 249 | 141 |
| CCAATTGGACTTAACATGT | 250 | 212 |
| CCTTCACTATGGTGCGCTA | 251 | 133 |
| GAGAACTCATGGTTTAACT | 252 | 126 |
| GGAGATTTCTCACGATGAA | 253 | 138 |
| CATTTGCTATGAACACGCA | 254 | 135 |
| GCCTTATTATGATCTTGTA | 255 | 127 |
| CTGTTCAAGAAAGAGGTGA | 256 | 145 |
| GAATTCTACTTCCCAACAT | 257 | 132 |
| CAGGTTACATATCAGTTGA | 258 | 151 |
| CCACCTATCCCTCTGGACA | 259 | 148 |
| CCAAGGAGCAGCGACTCAA | 260 | 143 |
| CACCTTTGAGCAATGGTCT | 261 | 131 |
| GCCAATATAGATCTGCTTA | 262 | 131 |
| GAGATTGGACTCTGCAGTA | 263 | 168 |
| GGAACTTAAGGAAACCTTT | 264 | 158 |
| GTATAATGCTAAAGACACA | 265 | 143 |
| GTGCATTTCGGGTAGGGAT | 266 | 205 |
| CATTGACAGATATTGGGCA | 267 | 143 |
| CTCAAAGGAGGTCATGTTT | 268 | 136 |
| GGATATTTGCCACAGCATT | 269 | 135 |
| CATTGAGTACTCTGGCATT | 270 | 164 |
| GAATTGGTGGCAGAACAGA | 271 | 133 |
| GCTCTTACCAGACAATCAA | 272 | 127 |
| CTAATGAACAGGTGTTGAA | 273 | 133 |
| CTACCTCGTGGGATCCCAT | 274 | 125 |
| CGCTCTACTTCTGCTTCGT | 275 | 134 |
| CTAAGAACATGATGTGGTA | 276 | 130 |
| CAGTCAAAGACATATGAGA | 277 | 126 |
| CTCAATGGAGGACTTGAAT | 278 | 133 |
| GATTATACCTTAACTATGT | 279 | 131 |
| CCATGCTAGTGGTCCTCAT | 280 | 137 |
| CTCATCAAATCCCGAGGGA | 281 | 133 |
| GACACTATAGTCGAGCCAA | 282 | 139 |
| CCTTAAGTCCAGCAACATA | 283 | 138 |
| GAGGAAATTGCCAGGATCA | 284 | 138 |
| GTCTCAAGGTACTTTACGA | 285 | 139 |
| GGTTATACAGGTTGTTAAA | 286 | 138 |
| CCTATTGGATCTCCCAGCT | 287 | 126 |
| GATCTTCGGAACTTAGTGA | 288 | 129 |
| CCATTAACCAGAGAATAGT | 289 | 149 |
| CTCGAAAGCAAAGAGATAA | 290 | 127 |
| GGAACATTCTAGAATCTGT | 291 | 127 |
| CACATTGAGAGAGTGTATA | 292 | 128 |
| CCATCATTTGTATTCCGGA | 293 | 135 |
| CAGTTCAAGGCTGCGCTCA | 294 | 136 |
| GGATTACAACCTGAGAGTT | 295 | 167 |
| CTTGTTGCCTCACATCCGA | 296 | 144 |
| GAAATTATTGGGCGTCATA | 297 | 128 |
| CCATCAAGCAGGGTTTCAA | 298 | 126 |
| GGAAATGATGACTTTATGT | 299 | 132 |
| GACAATTCAGAGAGTCACA | 300 | 136 |
| CCAATAATGTCACCTCGAA | 301 | 151 |
| GACCTAGATGTCATTGTTT | 302 | 175 |
| GAAGTTTGACCGCTACAAA | 303 | 128 |
| GTGTATGCCGCAACGGCTA | 309 | 143 |
| CAGCTAATCATCAACCCAA | 305 | 149 |
| CAATTTGAACCAAGAGTCT | 306 | 138 |
| CAGATACCGGGCCCAACGA | 307 | 139 |
| CAGTTCTCGATCACCGGTA | 308 | 132 |
| GGATCAACCTTCCTATCCA | 309 | 150 |
| CATATGATCTCCACCATGA | 310 | 147 |
| GTATGTGCGCTATTTCCGA | 311 | 143 |
| CAATACAGTTCCGCTTGTA | 312 | 132 |
| GTGAATCTCAACTACCGAA | 313 | 151 |
| GGAATACCTTTCATCGATT | 314 | 131 |
| GACCTATGATGCACCACAT | 315 | 127 |
| GGCAACAGTTCTTCATCGA | 316 | 139 |
| CCGGAAATACAGATTGATT | 317 | 132 |
| CGCGAGGTGCACTTCATGA | 318 | 170 |
| CCATGAACGCCGAGGACGA | 319 | 135 |
| CATTCTAAAGGTCAGTGTA | 320 | 127 |
| GATCATCTGCCTTCCGAAA | 321 | 214 |
| CCTACTTTGGCCTGCTTGA | 322 | 132 |
| CTTACTACAACAATCTGTA | 323 | 132 |
| GGAATATCTTCTAATGGGA | 324 | 133 |
| CGTGTTAGCCTTACACCAA | 325 | 153 |
| GTAATTGAGTATAAGTCCA | 326 | 133 |
| GTTTGACATTGAGATTGGA | 327 | 131 |
| CAGACTTCTTTGAGTACTT | 328 | 137 |
| CAGTGAATCAGGTGGCGAA | 329 | 137 |
| GGGATAAGCCTCCTTTCTT | 330 | 129 |
| CACAATGTGGTCCACATGA | 331 | 128 |
| GTACCTCTCCTATAGACAT | 332 | 162 |
| CGGTATTGATGATGTGCCT | 333 | 161 |
| CTTGAACTTCCACGAGGTA | 334 | 138 |
| CATAAGATTGGCTGGAATT | 335 | 211 |
| GCACCTACCTGGGTCACTT | 336 | 139 |
| CTTAGACTGGAAATTAAGA | 337 | 138 |
| GAGCTAAAGCTGTAACCCA | 338 | 186 |
| GGTGATAGGTAAGAATATT | 339 | 129 |
| GAGATGATGGACTCATGTA | 340 | 127 |
| CACCTTATATCCCAAATGT | 341 | 146 |
| CCTACGTCTTCAACATCAT | 342 | 131 |
| CATCAAGCAGGGTTTCAAA | 343 | 130 |

The 343 RNAi targets in Table 5 were further evaluated for their capabilities in improving exogenous polypeptide production. To avoid clone-specific impacts, two different production cell lines were used for this evaluation. For this round of evaluation, an siRNA molecule for each of the 343 RNAi targets were transfected into these two cell lines and the production levels were measured 5 days post transfection. For each siRNA and each cell line, triplicates were performed. Of the 343 siRNA molecules being tested, 75 of them were identified to be able to improve the productivity by at least 30% on average, or the improvements were statistically significant (Table 5B).

TABLE 5B
Robustness of increased mAb production by
CHO siRNA molecules

| | | Average of Relative |
|---|---|---|
| | SEQ | Production Levels (%) |
| RNAi Target | ID | in Two Different Cell |
| Sequence | NO | Lines |
| GAACTAGAGCGGTTTGTAA | 20 | 130 |
| CTTCCTTTAACAATCAGAT | 51 | 153 |
| GACTTATATCCAGGACAAA | 46 | 162 |
| GGATCAACAGGGTGTTGTT | 42 | 164 |
| GGTTTATCCAGGAAGTATA | 74 | 130 |
| CAGGAATCCTGGAGAGTAA | 58 | 184 |
| GGAGTATCATCTTGGGCTA | 110 | 132 |
| GTGAGTGTGTGATCTGCCT | 117 | 135 |
| GGATGATGAAGGTGTTCTT | 84 | 210 |
| CGATTTGGATAGCCAACAA | 111 | 139 |
| CCAAATATGGCTTGTACTT | 95 | 144 |
| CCACTTACTTCATGACCTA | 112 | 144 |
| GATTGCATGGCTGTTCAAT | 89 | 156 |
| CAGCTAGCATCGGGACAGA | 101 | 146 |
| GAATTGAGCCACAGAGTAA | 131 | 158 |
| GAAACTGAAGGCTTTCTTA | 132 | 132 |
| GAACTACACATCTAAGTTT | 133 | 132 |
| CACAGTTCATCTTCGACCA | 134 | 186 |
| CAAAGAAGTTTGCAGATTT | 135 | 158 |
| CAAGAAGGATTTGACCGTA | 177 | 121 |
| CTATCCAGATGGTCGCCAA | 182 | 145 |
| CCCTTTATCATTGAGTGGA | 170 | 182 |
| CTGCTATTTCCATCCATGA | 183 | 141 |
| CCTATACCTTCAATTGCCA | 184 | 140 |
| CTCTAGAGTGCCAATCATA | 157 | 120 |
| GAGATCTACCGATACACAT | 188 | 143 |
| CCGATTCTTGTATCATGGA | 149 | 136 |
| CTTATGAGCTGGTGATCGA | 199 | 157 |
| CTATGTGTGTGATCTTGAA | 193 | 136 |
| GGAGTCTGGTTTAACCTCA | 212 | 168 |
| CTACCAAACTTCGAGGATA | 195 | 222 |
| CATTGAGGACCTTCGTGTA | 210 | 181 |
| CCAATGATGGCCATTCAGT | 217 | 154 |
| CTAACAACCGGCTCCTCAA | 207 | 166 |
| CCTTCAACATTGACCGAAA | 218 | 183 |
| GCCATTATTCAGTGATGTT | 201 | 191 |
| GCAAGTTTGTGCCCTCAGA | 214 | 134 |
| CTGACAAAGAGCTTCCACT | 215 | 131 |
| CACTGTACCTCTAATTACA | 243 | 148 |
| GCTTTCAACTGTATGGTCT | 238 | 130 |
| CTCAGCAGTGGATGACCAA | 230 | 154 |
| GCAGTATGTGGCCAGTTAT | 227 | 165 |
| CCTTCTTCCTCTCCATGTA | 231 | 132 |
| GATGTCAAGTCATATTACA | 235 | 138 |
| CTTACTCTTCTCACATGGA | 223 | 171 |
| CAGCATTTGTCAAACGTCA | 228 | 170 |
| CTTGTAAGTGGGTCACCAA | 241 | 135 |
| GGAGATTTCTCACGATGAA | 253 | 182 |
| GGATATTTGCCACAGCATT | 269 | 148 |
| CCAAAGATGGCATTGACCA | 246 | 150 |
| GTGCATTTCGGGTAGGGAT | 266 | 137 |
| GCCAATATAGATCTGCTTA | 262 | 138 |
| CAGTCAAAGACATATGAGA | 277 | 133 |
| CCATGCTAGTGGTCCTCAT | 280 | 175 |
| GCTCTTACCAGACAATCAA | 272 | 168 |
| GGAACATTCTAGAATCTGT | 291 | 155 |
| GGTTATACAGGTTGTTAAA | 286 | 141 |
| CCATCATTTGTATTCCGGA | 293 | 145 |
| CTCGAAAGCAAAGAGATAA | 290 | 140 |
| CTAAGAACATGATGTGGTA | 276 | 146 |
| CCTATTGGATCTCCCAGCT | 287 | 153 |
| GACCTATGATGCACCACAT | 315 | 178 |
| CATATGATCTCCACCATGA | 310 | 162 |
| GATCATCTGCCTTCCGAAA | 321 | 137 |
| CCTACTTTGGCCTGCTTGA | 322 | 131 |
| GTATGTGCGCTATTTCCGA | 311 | 144 |
| GTGTATGCCGCAACGGCTA | 309 | 140 |
| CAATACAGTTCCGCTTGTA | 312 | 170 |
| CAGCTAATCATCAACCCAA | 305 | 161 |
| GTGAATCTCAACTACCGAA | 313 | 164 |
| CTTACTACAACAATCTGTA | 323 | 192 |
| CGGTATTGATGATGTGCCT | 333 | 130 |
| CCTACGTCTTCAACATCAT | 342 | 159 |
| GGTGATAGGTAAGAATATT | 339 | 154 |
| GTTTGACATTGAGATTGGA | 327 | 134 |

In order to identify RNAi targets that have universal impacts on exogenous polypeptide production, i.e., which is not specific to the clone, exogenous polypeptide or expression promoter, the siRNA molecules for each of the RNAi targets in Table 5B were further evaluated. During this round of evaluation, five different cell lines which produce at least three different proteins were used. Furthermore, these five different producers were generated using different expression systems, e.g. different selectable markers (puromycin or glutamine synthetase) and/or different promoters (cytomegalovirus or elongation factor 1 alpha). The rationale of including these varieties is to assure that the siRNA sequences identified have impacts on general protein expression, and can be used for multiple projects and expression systems. For each condition, multiple transfections were performed to assure the statistical significance. Of the 75 siRNA candidates, 11 siRNA were found to be able to improve the protein expression significantly in at least four out of the five different cell lines (Table 5C). Six of the eleven functioned in all five different cell lines and the other five worked in at least 4 out of the 5 cell lines.

TABLE 5C
Robustness of increased mAb
production by inhibiting specific siRNA
targets of the invention

| RNAi Target Sequence | SEQ ID | No. of Cell |
|---|---|---|
| (Table 3) | NO | Lines |
| GAGATCTACCGATACACAT | 188 | 4/5 |
| CCGATTCTTGTATCATGGA | 149 | 4/5 |
| CATTGAGGACCTTCGTGTA | 210 | 5/5 |
| CAGCATTTGTCAAACGTCA | 228 | 4/5 |
| GGAGATTTCTCACGATGAA | 253 | 4/5 |
| GGATATTTGCCACAGCATT | 269 | 4/5 |
| GCCAATATAGATCTGCTTA | 262 | 5/5 |
| GCTCTTACCAGACAATCAA | 272 | 5/5 |
| GGAACATTCTAGAATCTGT | 291 | 5/5 |
| GGTGATAGGTAAGAATATT | 339 | 5/5 |
| GTTTGACATTGAGATTGGA | 327 | 5/5 |

### Example 3. Effect of Inhibiting Expression of WNK Lysine Deficient Protein Kinase 4 (Wnk4) with a siRNA

Wnk4 (CHO accession number XM_003504666) is one of the top 11 RNAi target genes identified in the screening experiments described above. To further evaluate the impact of inhibiting expression of Wnk4 on production of an exogenous antibody, an established CHO cell line expressing a recombinant humanized monoclonal antibody against vascular endothelial growth factor A (VEGF-A) was transiently transfected with a siRNA targeting SEQ ID NO:327 using a standard lipofectamine transfection protocol. The transfected cell line and the untransfected cell line (the “control”) were cultured in Dulbecco's Modified Eagle Medium (DMEM) supplemented with 10% fetal bovine serum (FBS), 1× glutamine synthetase expression supplement (GSEM) and 2 mM glutamine in T-25 flasks and 6-well plates at 37 C with 5% CO₂

After 3 days of incubation, cells from T-25 flasks were harvested and RNA was extracted from the harvested cells. To measure the level of mRNAs for Wnk4 and the light chain and heavy chains of the hu mAb in the extracted RNA, reverse transcription and subsequent real time PCR were performed on the extracted RNA from the transfected and control cultures. After 5 days of culture in 6-well plates, the amount of mAb in the culture supernatant was determined using reverse phase HPLC.

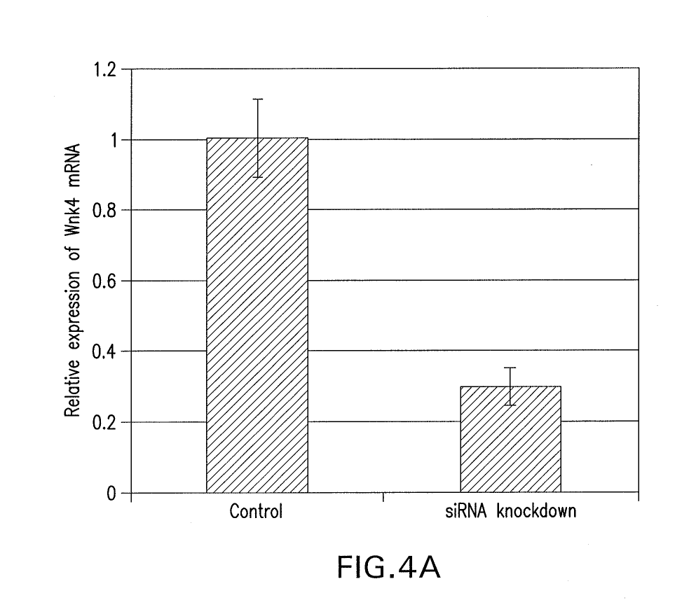

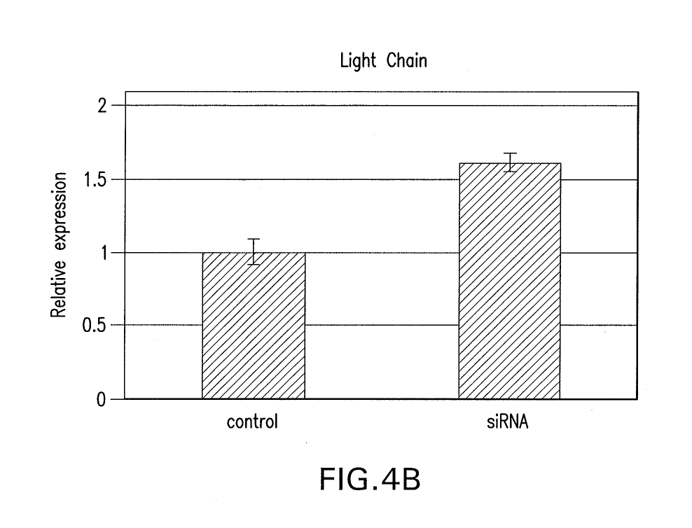

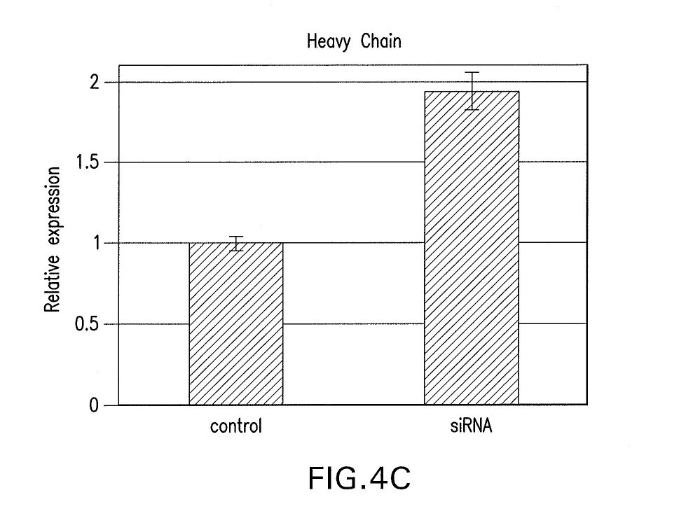

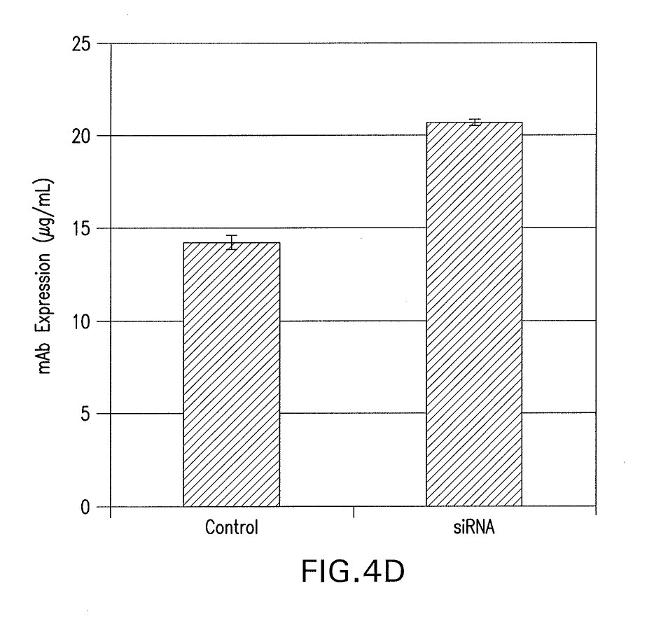

The level of Wnk4 mRNA in the siRNA-transfected culture was about 70% lower than in the control culture (FIG. 4A). In contrast, in the siRNA-transfected culture the mRNA levels for the exogenous mAb heavy and light chains were about 1.9 fold and 1.6 fold higher than in the control (FIG. 4B). Similarly, a 45% increase in mAb production was observed in the siRNA-transfected culture relative to the control (FIG. 4C).

### Example 4. Effect of Inhibiting Expression of Wnk4 Expression with a shRNA

To evaluate the long-term impact of inhibiting Wnk4 on target protein production in CHO cells, a cell line was created that was stably transfected with an expression vector that contained an expression cassette for a Wnk4 shRNA expression cassette and two expression cassettes for the light and heavy chains of a humanized anti-PD-1 mAb.

The expression vector backbone employed in the construction of the Wnk4 shRNA expressing cell line was the 9.4 kb pEE14 expression vector available from Lonza Ltd (Basel, Switzerland) and which contains: (1) a human CMV major immediate early promoter (hCMV-MIE), (2) a multiple cloning site (MCS), (3) a SV40 early poly A site (SV40 pA), (4) a Col E1 origin of replication (Col E1), (5) an ampicillin resistance gene (Amp-r) and (5) the SV40 late promoter (SV40 L), which drives the glutamine synthetase minigene (GS-minigene), see, e.g., US2002/0099183.

The shRNA expression cassette contained nucleotide sequences for a human U6 promoter, a shRNA targeting the Wnk4 gene, and a transcription terminator, and these nucleotide sequences are shown below.

U6 promoter (SEQ ID NO: 497):
CCCGAGTCCAACACCCGTGGGAATCCCATGGGCACCATGGCCCCTCGCTC
CAAAAATGCTTTCGCGTCGCGCAGACACTGCTCGGTAGTTTCGGGGATCA
GCGTTTGAGTAAGAGCCCGCGTCTGAACCCTCCGCGCCTCCCCGGCCCCA
GTGGAAAGACGCGCAGGCAAAACGCACCACGTGACGGAGCGTGACCGCGC
GCCGAGCGCGCGCCAAGGTCGGGCAGGAAGAGGGCCTATTTCCCATGATT
CCTTCATATTTGCATATACGATACAAGGCTGTTAGAGAGATAATTAGAAT
TAATTTGACTGTAAACACAAAGATATTAGTACAAAATACGTGACGTAGAA
AGTAATAATTTCTTGGGTAGTTTGCAGTTTTAAAATTATGTTTTAAAATG
GACTATCATATGCTTACCGTAACTTGAAAGTATTTCGATTTCTTGGCTTT
ATATATCTTGTGGAAAGGACGAAACACC
shRNA (SEQ ID NO: 498):
GATACCTCAAGTTTGACATTGAGATTGGAGTCTAGAGTCCAATCTCAATG
TCAAACTTGAGGTATC
Transcription Terminator:
TTTTT

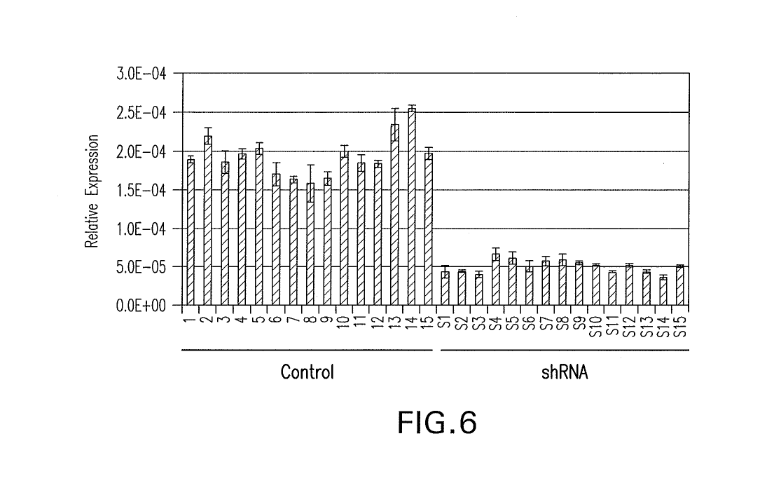

This shRNA expression cassette was inserted downstream of, and in opposite orientation to, the heavy chain mAb expression cassette, and a schematic of the resulting expression vector is shown in FIG. 5. A control expression vector was generated using the same mAb heavy and light chain expression cassettes, but the third expression cassette contained only the U6 promoter and terminator sequences. DNA for each of the shRNA and control vectors was linearized and transfected into CHOK1sv cells (Lonza, Ltd.), and stably transfected cell lines were generated using conventional selection and adaptation techniques. The top 15 mAb producing clones of the control and the shRNA expressing cell lines were selected for evaluation of Wnk4 mRNA expression in batch cultures. In brief, the selected clones were expanded into shake flasks and cultured in CD-CHO media 37° C. for 3 days, and then the cells were harvested, RNA extracted and Wnk4 mRNA levels, relative to expression of the housekeeping gene GAPDH, were measured as described in Example 3 above. As shown in FIG. 6, the clones from the shRNA transfected cell line exhibited an average of ˜30% lower expression of Wnk4 mRNA when compared to the control clones, which demonstrated Wnk4 mRNA knock-down as expected.

The effect of Wnk4 inhibition by shRNA on production of the anti-PD-1 mAb in stably transfected cells was further evaluated in batch (passage) cultures and fed batch cultures of the top 10 mAb producing clones from each of the shRNA transfected and control cell lines. For the batch cultures, the cell lines were cultured at 37° C., 5% CO₂ in CD CHO medium with 25 μM methionine sulfoximine (MSX) and passaged every 3-4 days with a seeding density of 2E5 cells per mL, and for the fed batch cultures, the cell lines were cultured at 37° C., 5% CO₂ in CD CHO medium (Thermo Fisher Scientific, Inc., Waltham, Mass. USA) for 14 days and a nutrient feed every 2-4 days that included amino acids, vitamin, nucleosides, hydrolysates and, as needed, glucose. The level of the anti-PD-1 mAb produced was measured by Protein A HPLC.

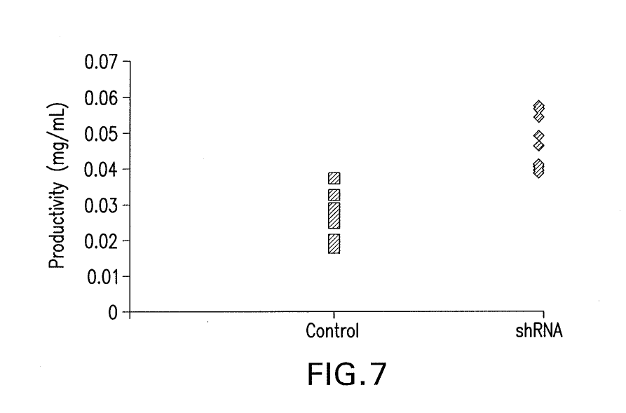

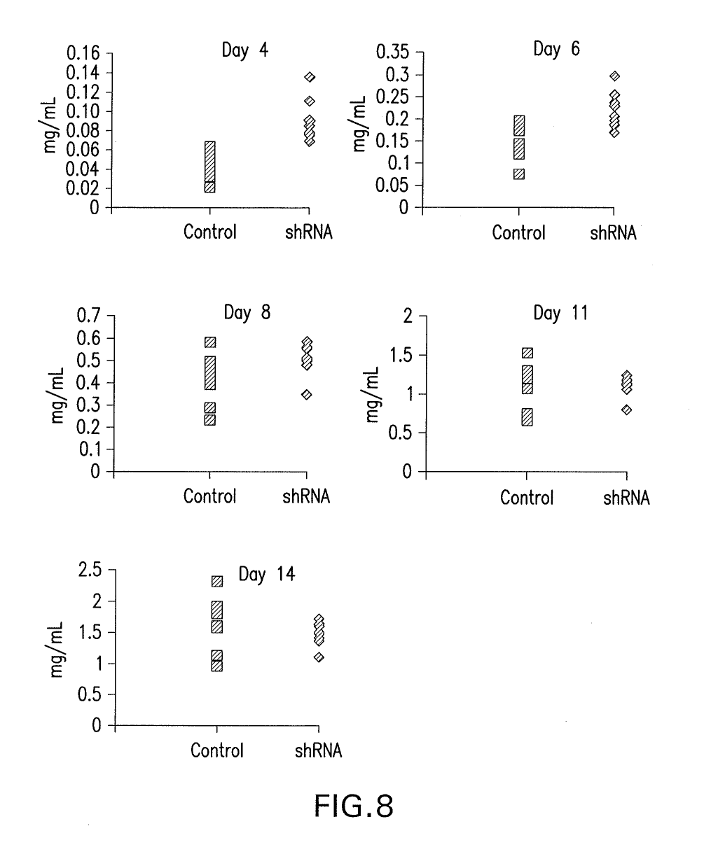

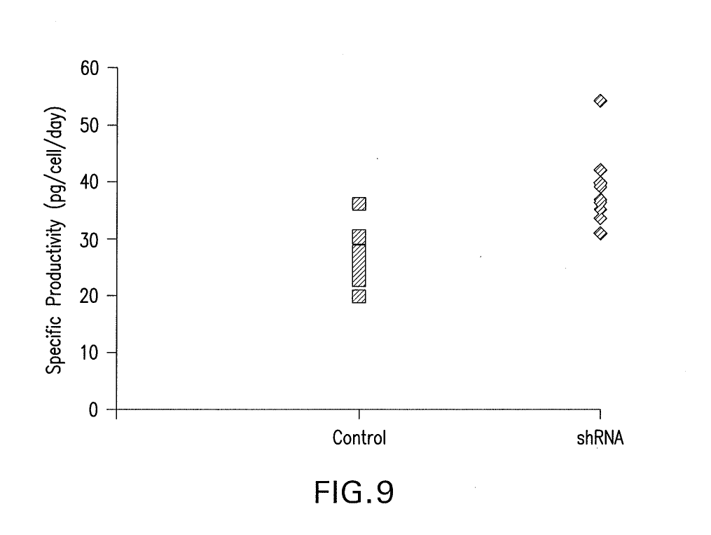

As shown in FIG. 7, the median volumetric productivity of the mAb after 3 days of batch (passage) culture of the shRNA transfected clones was ˜60% (p<0.001) greater than in the control clones. In the fed batch cultures, an initial improvement in volumetric productivity in the shRNA transfected clones was observed relative to the control clones, but this improvement diminished over time (FIG. 8). However, in the fed batch cultures, a ˜40% improvement (p<0.001) in the median specific productivity (Qp) in the shRNA transfected clones was observed after 14 culture days (FIG. 9).

1. A method of producing a polypeptide, which comprises
(a) providing a recombinant mammalian host cell capable of expressing the polypeptide;
(b) culturing the host cell under conditions suitable for
(i) effecting expression of the polypeptide and (ii) inhibiting expression of at least one RNAi target gene, and
(c) recovering the expressed polypeptide, wherein the RNAi target gene is a mouse gene selected from the group of mouse genes listed in the table immediately below or is an orthologue of the selected mouse gene.

| Gene | Mouse Gene |
|---|---|
| No. | (Accession No) |
| 1 | NM_024479 |
| 2 | NM_023697 |
| 3 | NM_013523 |
| 4 | NM_029840 |
| 5 | NM_023168 |
| 6 | NM_019980 |
| 7 | NM_013847 |
| 8 | NM_026871 |
| 9 | NM_133355 |
| 10 | NM_001003909 |
| 11 | NM_025350 |
| 12 | NM_025292 |
| 13 | NM_013415 |
| 14 | NM_001081414 |
| 15 | NM_133500 |
| 16 | NM_007958 |
| 17 | NM_021509 |
| 18 | NM_177941 |
| 19 | NM_080510 |
| 20 | NM_001033156 |
| 21 | NM_001081377 |
| 22 | NM_001014995 |
| 23 | NM_023543 |
| 24 | NM_026009 |
| 25 | NM_133684 |
| 26 | NM_021491 |
| 27 | NM_026984 |
| 28 | NM_023131 |
| 29 | NM_025650 |
| 30 | NM_011217 |
| 31 | NM_008048 |
| 32 | NM_025865 |
| 33 | NM_013855 |
| 34 | NM_025964 |
| 35 | NM_001004761 |
| 36 | NM_019828 |
| 37 | NM_011521 |
| 38 | NM_008737 |
| 39 | NM_008770 |
| 40 | NM_001024385 |
| 41 | NM_024267 |
| 42 | NM_008246 |
| 43 | NM_024454 |
| 44 | NM_008626 |
| 45 | NM_007647 |
| 46 | NM_011169 |
| 47 | NM_025485 |
| 48 | NM_199314 |
| 49 | NM_008206 |
| 50 | FJ042496 |
| 51 | NM_028052 |
| 52 | NM_024198 |
| 53 | NM_027880 |
| 54 | NM_028004 |
| 55 | NM_019688 |
| 56 | NM_028006 |
| 57 | NM_027880 |
| 58 | NM_133895 |
| 59 | NM_013563 |
| 60 | NM_013632 |
| 61 | NM_019724 |
| 62 | NM_013659 |
| 63 | NM_177909 |
| 64 | NM_175638 |
| 65 | NM_001081309 |
| 66 | NM_011101 |
| 67 | NM_029572 |
| 68 | NM_183294 |
| 69 | NM_199029 |
| 70 | NM_023517 |
| 71 | NM_024236 |
| 72 | NM_012048 |
| 73 | NM_020603 |
| 74 | NM_008957 |
| 75 | NM_138952 |
| 76 | NM_001001321 |
| 77 | NM_010588 |
| 78 | NM_172844 |
| 79 | NM_028450 |
| 80 | NM_030037 |
| 81 | NM_028475 |
| 82 | NM_019773 |
| 83 | NM_178076 |
| 84 | NM_028784 |
| 85 | NM_018852 |
| 86 | NM_007979 |
| 87 | NM_001037755 |
| 88 | NM_173388 |
| 89 | NM_133216 |
| 90 | NM_029947 |
| 91 | NM_001195413 |
| 92 | NM_001110327 |
| 93 | NM_019791 |
| 94 | NM_028175 |
| 95 | NM_028950 |
| 96 | NM_021426 |
| 97 | NM_033524 |
| 98 | NM_013815 |
| 99 | NM_028105 |
| 100 | NM_001014976 |
| 101 | NM_053208 |
| 102 | NM_026209 |
| 103 | NM_001111066 |
| 104 | NM_183315 |
| 105 | NM_134109 |
| 106 | NM_019972 |
| 107 | NM_030696 |
| 108 | NM_178682 |
| 109 | NM_146164 |
| 110 | NM_010484 |
| 111 | NM_010113 |
| 112 | NM_008720 |
| 113 | NM_029094 |
| 114 | NM_029097 |
| 115 | NM_030558 |
| 116 | NM_001039094 |
| 117 | NM_133206 |
| 118 | NM_010442 |
| 119 | NM_001080926 |
| 120 | NM_010402 |
| 121 | NM_010602 |
| 122 | NM_016781 |
| 123 | NM_011454 |
| 124 | NM_021607 |
| 125 | NM_029077 |
| 126 | NM_008770 |
| 127 | NM_021339 |
| 128 | NM_029582 |
| 129 | NM_027328 |
| 130 | NM_010111 |
| 131 | NM_010754 |
| 132 | NM_027838 |
| 133 | NM_028186 |
| 134 | NM_010413 |
| 135 | NM_026769 |
| 136 | NM_027088 |
| 137 | NM_018810 |
| 138 | NM_013880 |
| 139 | NM_008133 |
| 140 | NM_019827 |
| 141 | NM_009929 |
| 142 | NM_028803 |
| 143 | NM_029880 |
| 144 | NM_016792 |
| 145 | NM_010597 |
| 146 | NM_010865 |
| 147 | NM_008860 |
| 148 | XM_006502607 |
| 149 | NM_026536 |
| 150 | NM_145615 |
| 151 | NM_013630 |
| 152 | NM_008797 |
| 153 | NM_008171 |
| 154 | NM_173185 |
| 155 | NM_008842 |
| 156 | NM_134438 |
| 157 | NM_080644 |
| 158 | NM_007513 |
| 159 | NM_009714 |
| 160 | NM_007835 |
| 161 | NM_007659 |
| 162 | NM_145440 |
| 163 | NM_133728 |
| 164 | NM_001113352 |
| 165 | NM_007835 |
| 166 | NM_007878 |
| 167 | NM_007611 |
| 168 | NM_008032 |
| 169 | NM_007659 |
| 170 | NM_146170 |
| 171 | NM_011352 |
| 172 | NM_008772 |
| 173 | NM_007554 |
| 174 | NM_144783 |
| 175 | NM_007931 |
| 176 | NM_008020 |
| 177 | NM_008792 |
| 178 | NM_134160 |
| 179 | NM_134066 |
| 180 | NM_001110204 |
| 181 | NM_008277 |
| 182 | NM_001110208 |
| 183 | NM_008832 |
| 184 | NM_030688 |
| 185 | NM_008426 |
| 186 | NM_030677 |
| 187 | NM_023057 |
| 188 | NM_009993 |
| 189 | NM_172903 |
| 190 | NM_145542 |
| 191 | NM_028882 |
| 192 | NM_026719 |
| 193 | NM_010924 |
| 194 | NM_134028 |
| 195 | NM_054085 |
| 196 | NM_008525 |
| 197 | NM_173071 |
| 198 | NM_029706 |
| 199 | NM_001098170 |
| 200 | NM_178749 |
| 201 | NM_145458 |
| 202 | NM_001105196 |
| 203 | NM_146041 |
| 204 | NM_026482 |
| 205 | NM_145608 |
| 206 | NM_144954 |
| 207 | NM_178907 |
| 208 | NM_172152 |
| 209 | NM_148931 |
| 210 | NM_177185 |
| 211 | NM_175540 |
| 212 | NM_008437 |
| 213 | NM_026406 |
| 214 | NM_146177 |
| 215 | NM_144893 |
| 216 | NM_029294 |
| 217 | NM_178396 |
| 218 | NM_145922 |
| 219 | NM_152804 |
| 220 | NM_212433 |
| 221 | NM_001110497 |
| 222 | NM_021516 |
| 223 | NM_146096 |
| 224 | NM_008174 |
| 225 | NM_011322 |
| 226 | NM_008846 |
| 227 | NM_028850 |
| 228 | NM_173379 |
| 229 | NM_133819 |
| 230 | NM_033573 |
| 231 | NM_139142 |
| 232 | NM_177357 |
| 233 | NM_010957 |
| 234 | NM_009469 |
| 235 | NM_011201 |
| 236 | NM_178746 |
| 237 | NM_008518 |
| 238 | NM_029020 |
| 239 | NM_011846 |
| 240 | NM_144792 |
| 241 | NM_177725 |
| 242 | NM_029005 |
| 243 | NM_198246 |
| 244 | NM_033269 |
| 245 | NM_019513 |
| 246 | NM_148930 |
| 247 | NM_198214 |
| 248 | NM_001029842 |
| 249 | NM_009157 |
| 250 | NM_146003 |
| 251 | NM_029536 |
| 252 | NM_018736 |
| 253 | NM_001081206 |
| 254 | NM_021360 |
| 255 | NM_009370 |
| 256 | NM_013571 |
| 257 | NM_001081306 |
| 258 | NM_144902 |
| 259 | NM_016689 |
| 260 | NM_019719 |
| 261 | NM_019653 |
| 262 | NM_016697 |
| 263 | NM_175025 |
| 264 | NM_016708 |
| 265 | NM_001081315 |
| 266 | NM_013540 |
| 267 | NM_145700 |
| 268 | NM_009097 |
| 269 | NM_145355 |
| 270 | NM_029274 |
| 271 | NM_019802 |
| 272 | NM_153792 |
| 273 | NM_010568 |
| 274 | NM_175087 |
| 275 | NM_199251 |
| 276 | NM_177646 |
| 277 | NM_146086 |
| 278 | NM_146239 |
| 279 | NM_001038701 |
| 280 | NM_144834 |
| 281 | NM_145121 |
| 282 | NM_019827 |
| 283 | NM_177395 |
| 284 | NM_175127 |
| 285 | NM_021390 |
| 286 | NM_138606 |
| 287 | NM_001109045 |
| 288 | NM_016744 |
| 289 | NM_010154 |
| 290 | NM_009595 |
| 291 | NM_021485 |
| 292 | NM_199446 |
| 293 | NM_011083 |
| 294 | NM_001033254 |
| 295 | NM_153135 |
| 296 | NM_011903 |
| 297 | NM_001033209 |
| 298 | NM_213733 |
| 299 | NM_023209 |
| 300 | NM_207683 |
| 301 | NM_010899 |
| 302 | NM_011658 |
| 303 | NM_011716 |
| 304 | NM_010142 |
| 305 | NM_010934 |
| 306 | NM_011815 |
| 307 | NM_018810 |
| 308 | NM_133882 |
| 309 | NM_010043 |
| 310 | NM_001033328 |
| 311 | NM_177648 |
| 312 | NM_010434 |
| 313 | NM_011706 |
| 314 | NM_028779 |
| 315 | NM_175514 |
| 316 | NM_008381 |
| 317 | NM_021565 |
| 318 | NM_010205 |
| 319 | NM_010608 |
| 320 | NM_010728 |
| 321 | NM_133897 |
| 322 | NM_025968 |
| 323 | NM_015826 |
| 324 | NM_009011 |
| 325 | NM_025846 |
| 326 | NM_011218 |
| 327 | NM_175638 |
| 328 | NM_010794 |
| 329 | NM_177992 |
| 330 | NM_175465 |
| 331 | NM_207210 |
| 332 | NM_009809 |
| 333 | NM_001008702 |
| 334 | NM_011846 |
| 335 | NM_177353 |
| 336 | NM_198108 |
| 337 | NM_011077 |
| 338 | NM_021516 |
| 339 | NM_199159 |
| 340 | NM_023383 |
| 341 | NM_001037758 |
| 342 | NM_016975 |
| 343 | NM_213733 |

2. The method of claim 1, wherein the RNAi target gene is a mouse gene or a Chinese hamster ovary (CHO) gene selected from the group of genes listed in the table immediately below or is an orthologue of the selected mouse or CHO gene.

| Mouse RNAi Target | CHO RNAi Target |
|---|---|
| Gene | Gene |
| (Accession No.) | (Accession No.) |
| NM_001033156 | XM_003511078 |
| NM_028052 | XM_003503668.1 |
| NM_011169 | XM_003504158 |
| NM_008246 | XM_003513214 |
| NM_008957 | XM_003514770 |
| NM_133895 | XM_003505358 |
| NM_010484 | XM_003506082 |
| NM_133206 | XM_003510170 |
| NM_028784 | XM_003497479 |
| NM_010113 | XM_007653160 |
| NM_028950 | XM_003503382 |
| NM_008720 | NM_001246687 |
| NM_133216 | XM_003511942 |
| NM_053208 | XM_003505261 |
| NM_010754 | XM_003501037, |
| | XM_003501038 |
| NM_027838 | XR_135851 |
| NM_028186 | XM_003515516 |
| NM_010413 | XM_003505101 |
| NM_026769 | XM_003512003 |
| NM_008792 | XM_003508113 |
| NM_001110208 | XM_003505253 |
| NM_146170 | XM_003507962, |
| | XM_003507963 |
| NM_008832 | XM_003512672, |
| | XM_003512673 |
| NM_030688 | XM_003507545 |
| NM_080644 | NW_006879584 |
| NM_009993 | XM_003502400 |
| NM_026536 | XM_003511591 |
| NM_001098170 | XM_003495474 |
| NM_010924 | XM_003509317 |
| NM_008437 | XM_003510897 |
| NM_054085 | XM_003511682 |
| NM_177185 | XM_003503221 |
| NM_178396 | XM_003499339 |
| NM_178907 | XM_003500390 |
| NM_145922 | XM_003514579 |
| NM_145458 | XM_003502752 |
| NM_146177 | XM_003508241 |
| NM_144893 | NM_001243991 |
| NM_198246 | XM_003513347 |
| NM_029020 | XM_003501302 |
| NM_033573 | XM_003502900 |
| NM_028850 | XM_003513492 |
| NM_139142 | XM_003507723 |
| NM_011201 | XM_003502483 |
| NM_146096 | XM_003511733 |
| NM_173379 | XM_007642431 |
| NM_177725 | XM_003513970 |
| NM_001081206 | XM_003500967 |
| NM_145355 | XM_003509299 |
| NM_148930 | XM_003500410 |
| NM_013540 | XM_003500211, |
| | XM_003500212 |
| NM_016697 | XM_007643638 |
| NM_146086 | XM_003506313 |
| NM_144834 | XM_003509378 |
| NM_153792 | XM_003501507, |
| | XM_003501508 |
| NM_021485 | XM_003509935 |
| NM_138606 | XM_003505096 |
| NM_011083 | XM_003504810 |
| NM_009595 | XM_003507470, |
| | XM_003507471 |
| NM_177646 | XM_003512693 |
| NM_001109045 | XM_003514321 |
| NM_175514 | XM_003503395 |
| NM_001033328 | XM_007653510 |
| NM_133897 | XM_003506392 |
| NM_025968 | XM_003510575 |
| NM_177648 | XM_003513968 |
| NM_010142 | XM_003503638 |
| NM_010434 | XM_003497435, |
| | XM_003497436 |
| NM_010934 | XM_003510777 |
| NM_011706 | XM_003496342, |
| | XM_003496343 |
| NM_015826 | XM_003514815 |
| NM_001008702 | XM_003503098 |
| NM_016975 | XM_003500430 |
| NM_199159 | XM_003504375 |
| NM_175638 | XM_003504666 |

3. The method of claim 1, wherein the RNAi target gene is a mammalian gene selected from the group of mouse and CHO genes listed in the table immediately below or is an ortholog of the selected mouse or CHO gene.

| Mouse RNAi Target | CHO RNAi Target |
|---|---|
| Gene | Gene |
| (Accession No.) | (Accession No.) |
| NM_009993 | XM_003502400 |
| NM_026536 | XM_003511591 |
| NM_177185 | XM_003503221 |
| NM_173379 | XM_007642431 |
| NM_001081206 | XM_003500967 |
| NM_145355 | XM_003509299 |
| NM_016697 | XM_007643638 |
| NM_153792 | XM_003501507, |
| | XM_003501508 |
| NM_021485 | XM_003509935 |
| NM_199159 | XM_003504375 |
| NM_175638 | XM_003504666 |

4. The method of claim 1, wherein the culturing conditions that inhibit expression of the RNAi target gene comprise the presence of a short interfering nucleic acid (siNA) molecule for the selected RNAi target gene.

5. The method of claim 4, wherein the siNA molecule is an siRNA molecule that was transfected into the host cell before or during the culturing step.

6. The method of claim 5, wherein the siNA molecule is selected from the siRNA molecules shown in the table immediately below.

| RNAi | | |
|---|---|---|
| Target | | |
| Se- | | |
| quence | siRNA | |
| ID NO. | ANTISENSE SEQUENCE | SENSE SEQUENCE |
| 20 | UUACAAACCGCUCUAGUUCTT | GAACUAGAGCGGUUUGUAATT |
| | (SEQ ID NO: 344) | (SEQ ID NO: 345) |
| 51 | AUCUGAUUGUUAAAGGAAGTT | CUUCCUUUAACAAUCAGAUTT |
| | (SEQ ID NO: 346) | (SEQ ID NO: 347) |
| 46 | UUUGUCCUGGAUAUAAGUCTT | GACUUAUAUCCAGGACAAATT |
| | (SEQ ID NO: 348) | (SEQ ID NO: 349) |
| 42 | AACAACACCCUGUUGAUCCTT | GGAUCAACAGGGUGUUGUUTT |
| | (SEQ ID NO: 350) | (SEQ ID NO: 351) |
| 74 | UAUACUUCCUGGAUAAACCTT | GGUUUAUCCAGGAAGUAUATT |
| | (SEQ ID NO: 352) | (SEQ ID NO: 353) |
| 58 | UUACUCUCCAGGAUUCCUGTT | CAGGAAUCCUGGAGAGUAATT |
| | (SEQ ID NO: 354) | (SEQ ID NO: 355) |
| 110 | UAGCCCAAGAUGAUACUCCTT | GGAGUAUCAUCUUGGGCUATT |
| | (SEQ ID NO: 356) | (SEQ ID NO: 357) |
| 117 | AGGCAGAUCACACACUCACTT | GUGAGUGUGUGAUCUGCCUTT |
| | (SEQ ID NO: 358) | (SEQ ID NO: 359) |
| 84 | AAGAACACCUUCAUCAUCCTT | GGAUGAUGAAGGUGUUCUUTT |
| | (SEQ ID NO: 360) | (SEQ ID NO: 361) |
| 111 | UUGUUGGCUAUCCAAAUCGTT | CGAUUUGGAUAGCCAACAATT |
| | (SEQ ID NO: 362) | (SEQ ID NO: 363) |
| 95 | AAGUACAAGCCAUAUUUGGTT | CCAAAUAUGGCUUGUACUUTT |
| | (SEQ ID NO: 364) | (SEQ ID NO: 365) |
| 112 | UAGGUCAUGAAGUAAGUGGTT | CCACUUACUUCAUGACCUATT |
| | (SEQ ID NO: 366) | (SEQ ID NO: 367) |
| 89 | AUUGAACAGCCAUGCAAUCTT | GAUUGCAUGGCUGUUCAAUTT |
| | (SEQ ID NO: 368) | (SEQ ID NO: 369) |
| 101 | UCUGUCCCGAUGCUAGCUGTT | CAGCUAGCAUCGGGACAGATT |
| | (SEQ ID NO: 370) | (SEQ ID NO: 371) |
| 131 | UUACUCUGUGGCUCAAUUCTT | GAAUUGAGCCACAGAGUAATT |
| | (SEQ ID NO: 372) | (SEQ ID NO: 373) |
| 132 | UAAGAAAGCCUUCAGUUUCTT | GAAACUGAAGGCUUUCUUATT |
| | (SEQ ID NO: 374) | (SEQ ID NO: 375) |
| 133 | AAACUUAGAUGUGUAGUUCTT | GAACUACACAUCUAAGUUUTT |
| | (SEQ ID NO: 376) | (SEQ ID NO: 377) |
| 134 | UGGUCGAAGAUGAACUGUGTT | CACAGUUCAUCUUCGACCATT |
| | (SEQ ID NO: 378) | (SEQ ID NO: 379) |
| 135 | AAAUCUGCAAACUUCUUUGTT | CAAAGAAGUUUGCAGAUUUTT |
| | (SEQ ID NO: 380) | (SEQ ID NO: 381) |
| 177 | UACGGUCAAAUCCUUCUUGTT | CAAGAAGGAUUUGACCGUATT |
| | (SEQ ID NO: 382) | (SEQ ID NO: 383) |
| 182 | UUGGCGACCAUCUGGAUAGTT | CUAUCCAGAUGGUCGCCAATT |
| | (SEQ ID NO: 384) | (SEQ ID NO: 385) |
| 170 | UCCACUCAAUGAUAAAGGGTT | CCCUUUAUCAUUGAGUGGATT |
| | (SEQ ID NO: 386) | (SEQ ID NO: 387) |
| 183 | UCAUGGAUGGAAAUAGCAGTT | CUGCUAUUUCCAUCCAUGATT |
| | (SEQ ID NO: 388) | (SEQ ID NO: 389) |
| 184 | UGGCAAUUGAAGGUAUAGGTT | CCUAUACCUUCAAUUGCCATT |
| | (SEQ ID NO: 390) | (SEQ ID NO: 391) |
| 157 | UAUGAUUGGCACUCUAGAGTT | CUCUAGAGUGCCAAUCAUATT |
| | (SEQ ID NO: 392) | (SEQ ID NO: 393) |
| 188 | AUGUGUAUCGGUAGAUCUCTT | GAGAUCUACCGAUACACAUTT |
| | (SEQ ID NO: 394) | (SEQ ID NO: 395) |
| 149 | UCCAUGAUACAAGAAUCGGTT | CCGAUUCUUGUAUCAUGGATT |
| | (SEQ ID NO: 396) | (SEQ ID NO: 397) |
| 199 | UCGAUCACCAGCUCAUAAGTT | CUUAUGAGCUGGUGAUCGATT |
| | (SEQ ID NO: 398) | (SEQ ID NO: 399) |
| 193 | UUCAAGAUCACACACAUAGTT | CUAUGUGUGUGAUCUUGAATT |
| | (SEQ ID NO: 400) | (SEQ ID NO: 401) |
| 212 | UGAGGUUAAACCAGACUCCTT | GGAGUCUGGUUUAACCUCATT |
| | (SEQ ID NO: 402) | (SEQ ID NO: 403) |
| 195 | UAUCCUCGAAGUUUGGUAGTT | CUACCAAACUUCGAGGAUATT |
| | (SEQ ID NO: 404) | (SEQ ID NO: 405) |
| 210 | UACACGAAGGUCCUCAAUGTT | CAUUGAGGACCUUCGUGUATT |
| | (SEQ ID NO: 406) | (SEQ ID NO: 407) |
| 217 | ACUGAAUGGCCAUCAUUGGTT | CCAAUGAUGGCCAUUCAGUTT |
| | (SEQ ID NO: 408) | (SEQ ID NO: 409) |
| 207 | UUGAGGAGCCGGUUGUUAGTT | CUAACAACCGGCUCCUCAATT |
| | (SEQ ID NO: 410) | (SEQ ID NO: 411) |
| 218 | UUUCGGUCAAUGUUGAAGGTT | CCUUCAACAUUGACCGAAATT |
| | (SEQ ID NO: 412) | (SEQ ID NO: 413) |
| 201 | AACAUCACUGAAUAAUGGCTT | GCCAUUAUUCAGUGAUGUUTT |
| | (SEQ ID NO: 414) | (SEQ ID NO: 415) |
| 214 | UCUGAGGGCACAAACUUGCTT | GCAAGUUUGUGCCCUCAGATT |
| | (SEQ ID NO: 416) | (SEQ ID NO: 417) |
| 215 | AGUGGAAGCUCUUUGUCAGTT | CUGACAAAGAGCUUCCACUTT |
| | (SEQ ID NO: 418) | (SEQ ID NO: 419) |
| 243 | UGUAAUUAGAGGUACAGUGTT | CACUGUACCUCUAAUUACATT |
| | (SEQ ID NO: 420) | (SEQ ID NO: 421) |
| 238 | AGACCAUACAGUUGAAAGCTT | GCUUUCAACUGUAUGGUCUTT |
| | (SEQ ID NO: 422) | (SEQ ID NO: 423) |
| 230 | UUGGUCAUCCACUGCUGAGTT | CUCAGCAGUGGAUGACCAATT |
| | (SEQ ID NO: 424) | (SEQ ID NO: 425) |
| 227 | AUAACUGGCCACAUACUGCTT | GCAGUAUGUGGCCAGUUAUTT |
| | (SEQ ID NO: 426) | (SEQ ID NO: 427) |
| 231 | UACAUGGAGAGGAAGAAGGTT | CCUUCUUCCUCUCCAUGUATT |
| | (SEQ ID NO: 428) | (SEQ ID NO: 429) |
| 235 | UGUAAUAUGACUUGACAUCTT | GAUGUCAAGUCAUAUUACATT |
| | (SEQ ID NO: 430) | (SEQ ID NO: 431) |
| 223 | UCCAUGUGAGAAGAGUAAGTT | CUUACUCUUCUCACAUGGATT |
| | (SEQ ID NO: 432) | (SEQ ID NO: 433) |
| 228 | UGACGUUUGACAAAUGCUGTT | CAGCAUUUGUCAAACGUCATT |
| | (SEQ ID NO: 434) | (SEQ ID NO: 435) |
| 241 | UUGGUGACCCACUUACAAGTT | CUUGUAAGUGGGUCACCAATT |
| | (SEQ ID NO: 436) | (SEQ ID NO: 437) |
| 253 | UUCAUCGUGAGAAAUCUCCTT | GGAGAUUUCUCACGAUGAATT |
| | (SEQ ID NO: 438) | (SEQ ID NO: 439) |
| 269 | AAUGCUGUGGCAAAUAUCCTT | GGAUAUUUGCCACAGCAUUTT |
| | (SEQ ID NO: 440) | (SEQ ID NO: 441) |
| 246 | UGGUCAAUGCCAUCUUUGGTT | CCAAAGAUGGCAUUGACCATT |
| | (SEQ ID NO: 442) | (SEQ ID NO: 443) |
| 266 | AUCCCUACCCGAAAUGCACTT | GUGCAUUUCGGGUAGGGAUTT |
| | (SEQ ID NO: 444) | (SEQ ID NO: 445) |
| 262 | UAAGCAGAUCUAUAUUGGCTT | GCCAAUAUAGAUCUGCUUATT |
| | (SEQ ID NO: 446) | (SEQ ID NO: 447) |
| 277 | UCUCAUAUGUCUUUGACUGTT | CAGUCAAAGACAUAUGAGATT |
| | (SEQ ID NO: 448) | (SEQ ID NO: 449) |
| 280 | AUGAGGACCACUAGCAUGGTT | CCAUGCUAGUGGUCCUCAUTT |
| | (SEQ ID NO: 450) | (SEQ ID NO: 451) |
| 272 | UUGAUUGUCUGGUAAGAGCTT | GCUCUUACCAGACAAUCAATT |
| | (SEQ ID NO: 452) | (SEQ ID NO: 453) |
| 291 | ACAGAUUCUAGAAUGUUCCTT | GGAACAUUCUAGAAUCUGUTT |
| | (SEQ ID NO: 454) | (SEQ ID NO: 455) |
| 286 | UUUAACAACCUGUAUAACCTT | GGUUAUACAGGUUGUUAAATT |
| | (SEQ ID NO: 456) | (SEQ ID NO: 457) |
| 293 | UCCGGAAUACAAAUGAUGGTT | CCAUCAUUUGUAUUCCGGATT |
| | (SEQ ID NO: 458) | (SEQ ID NO: 459) |
| 290 | UUAUCUCUUUGCUUUCGAGTT | CUCGAAAGCAAAGAGAUAATT |
| | (SEQ ID NO: 460) | (SEQ ID NO: 461) |
| 276 | UACCACAUCAUGUUCUUAGTT | CUAAGAACAUGAUGUGGUATT |
| | (SEQ ID NO: 462) | (SEQ ID NO: 463) |
| 287 | AGCUGGGAGAUCCAAUAGGTT | CCUAUUGGAUCUCCCAGCUTT |
| | (SEQ ID NO: 464) | (SEQ ID NO: 465) |
| 315 | AUGUGGUGCAUCAUAGGUCTT | GACCUAUGAUGCACCACAUTT |
| | (SEQ ID NO: 466) | (SEQ ID NO: 467) |
| 310 | UCAUGGUGGAGAUCAUAUGTT | CAUAUGAUCUCCACCAUGATT |
| | (SEQ ID NO: 468) | (SEQ ID NO: 469) |
| 321 | UUUCGGAAGGCAGAUGAUCTT | GAUCAUCUGCCUUCCGAAATT |
| | (SEQ ID NO: 470) | (SEQ ID NO: 471) |
| 322 | UCAAGCAGGCCAAAGUAGGTT | CCUACUUUGGCCUGCUUGATT |
| | (SEQ ID NO: 472) | (SEQ ID NO: 473) |
| 311 | UCGGAAAUAGCGCACAUACTT | GUAUGUGCGCUAUUUCCGATT |
| | (SEQ ID NO: 474) | (SEQ ID NO: 475) |
| 304 | UAGCCGUUGCGGCAUACACTT | GUGUAUGCCGCAACGGCUATT |
| | (SEQ ID NO: 476) | (SEQ ID NO: 477) |
| 312 | UACAAGCGGAACUGUAUUGTT | CAAUACAGUUCCGCUUGUATT |
| | (SEQ ID NO: 478) | (SEQ ID NO: 479) |
| 305 | UUGGGUUGAUGAUUAGCUGTT | CAGCUAAUCAUCAACCCAATT |
| | (SEQ ID NO: 480) | (SEQ ID NO: 481) |
| 313 | UUCGGUAGUUGAGAUUCACTT | GUGAAUCUCAACUACCGAATT |
| | (SEQ ID NO: 482) | (SEQ ID NO: 483) |
| 323 | UACAGAUUGUUGUAGUAAGTT | CUUACUACAACAAUCUGUATT |
| | (SEQ ID NO: 484) | (SEQ ID NO: 485) |
| 333 | AGGCACAUCAUCAAUACCGTT | CGGUAUUGAUGAUGUGCCUTT |
| | (SEQ ID NO: 486) | (SEQ ID NO: 487) |
| 342 | AUGAUGUUGAAGACGUAGGTT | CCUACGUCUUCAACAUCAUTT |
| | (SEQ ID NO: 488) | (SEQ ID NO: 489) |
| 339 | AAUAUUCUUACCUAUCACCTT | GGUGAUAGGUAAGAAUAUUTT |
| | (SEQ ID NO: 490) | (SEQ ID NO: 491) |
| 327 | UCCAAUCUCAAUGUCAAACTT | GUUUGACAUUGAGAUUGGATT |
| | (SEQ ID NO: 492) | (SEQ ID NO: 493) |

7. The method of claim 5, wherein the siRNA molecule inhibits the expression of an RNAi target sequence selected from the group consisting of SEQ ID NO:188, SEQ ID NO:149, SEQ ID NO:210, SEQ ID NO:228, SEQ ID NO:253, SEQ ID NO:269, SEQ ID NO:262, SEQ ID NO:272, SEQ ID NO:291, SEQ ID NO:339 and SEQ ID NO:327.

8. The method of claim 7, wherein the siRNA molecule comprises the antisense and sense sequence pair shown in the Table immediately below for the selected RNAi target sequence.

| RNAi | | |
|---|---|---|
| Target | | |
| Se- | | |
| quence | siRNA | |
| ID NO. | ANTISENSE SEQUENCE | SENSE SEQUENCE |
| 188 | AUGUGUAUCGGUAGAUCUCTT | GAGAUCUACCGAUACACAUTT |
| | (SEQ ID NO: 394) | (SEQ ID NO: 395) |
| 149 | UCCAUGAUACAAGAAUCGGTT | CCGAUUCUUGUAUCAUGGATT |
| | (SEQ ID NO: 396) | (SEQ ID NO: 397) |
| 210 | UACACGAAGGUCCUCAAUGTT | CAUUGAGGACCUUCGUGUATT |
| | (SEQ ID NO: 406) | (SEQ ID NO: 407) |
| 228 | UGACGUUUGACAAAUGCUGTT | CAGCAUUUGUCAAACGUCATT |
| | (SEQ ID NO: 434) | (SEQ ID NO: 435) |
| 253 | UUCAUCGUGAGAAAUCUCCTT | GGAGAUUUCUCACGAUGAATT |
| | (SEQ ID NO: 438) | (SEQ ID NO: 439) |
| 269 | AAUGCUGUGGCAAAUAUCCTT | GGAUAUUUGCCACAGCAUUTT |
| | (SEQ ID NO: 440) | (SEQ ID NO: 441) |
| 262 | UAAGCAGAUCUAUAUUGGCTT | GCCAAUAUAGAUCUGCUUATT |
| | (SEQ ID NO: 446) | (SEQ ID NO: 447) |
| 272 | UUGAUUGUCUGGUAAGAGCTT | GCUCUUACCAGACAAUCAATT |
| | (SEQ ID NO: 452) | (SEQ ID NO: 453) |
| 291 | ACAGAUUCUAGAAUGUUCCTT | GGAACAUUCUAGAAUCUGUTT |
| | (SEQ ID NO: 454) | (SEQ ID NO: 455) |
| 339 | AAUAUUCUUACCUAUCACCTT | GGUGAUAGGUAAGAAUAUUTT |
| | (SEQ ID NO: 490) | (SEQ ID NO: 491) |
| 327 | UCCAAUCUCAAUGUCAAACTT | GUUUGACAUUGAGAUUGGATT |
| | (SEQ ID NO: 492) | (SEQ ID NO: 493) |

9.-12. (canceled)

13. An expression vector which comprises at least one expression cassette that is capable of expressing an shRNA in a mammalian host cell to inhibit expression of an RNAi target gene which is a mouse gene selected from the group of mouse genes listed in the table immediately below or is an ortholog of the selected mouse gene.

| Gene | Mouse Gene |
|---|---|
| No. | (Accession No) |
| 1 | NM_024479 |
| 2 | NM_023697 |
| 3 | NM_013523 |
| 4 | NM_029840 |
| 5 | NM_023168 |
| 6 | NM_019980 |
| 7 | NM_013847 |
| 8 | NM_026871 |
| 9 | NM_133355 |
| 10 | NM_001003909 |
| 11 | NM_025350 |
| 12 | NM_025292 |
| 13 | NM_013415 |
| 14 | NM_001081414 |
| 15 | NM_133500 |
| 16 | NM_007958 |
| 17 | NM_021509 |
| 18 | NM_177941 |
| 19 | NM_080510 |
| 20 | NM_001033156 |
| 21 | NM_001081377 |
| 22 | NM_001014995 |
| 23 | NM_023543 |
| 24 | NM_026009 |
| 25 | NM_133684 |
| 26 | NM_021491 |
| 27 | NM_026984 |
| 28 | NM_023131 |
| 29 | NM_025650 |
| 30 | NM_011217 |
| 31 | NM_008048 |
| 32 | NM_025865 |
| 33 | NM_013855 |
| 34 | NM_025964 |
| 35 | NM_001004761 |
| 36 | NM_019828 |
| 37 | NM_011521 |
| 38 | NM_008737 |
| 39 | NM_008770 |
| 40 | NM_001024385 |
| 41 | NM_024267 |
| 42 | NM_008246 |
| 43 | NM_024454 |
| 44 | NM_008626 |
| 45 | NM_007647 |
| 46 | NM_011169 |
| 47 | NM_025485 |
| 48 | NM_199314 |
| 49 | NM_008206 |
| 50 | FJ042496 |
| 51 | NM_028052 |
| 52 | NM_024198 |
| 53 | NM_027880 |
| 54 | NM_028004 |
| 55 | NM_019688 |
| 56 | NM_028006 |
| 57 | NM_027880 |
| 58 | NM_133895 |
| 59 | NM_013563 |
| 60 | NM_013632 |
| 61 | NM_019724 |
| 62 | NM_013659 |
| 63 | NM_177909 |
| 64 | NM_175638 |
| 65 | NM_001081309 |
| 66 | NM_011101 |
| 67 | NM_029572 |
| 68 | NM_183294 |
| 69 | NM_199029 |
| 70 | NM_023517 |
| 71 | NM_024236 |
| 72 | NM_012048 |
| 73 | NM_020603 |
| 74 | NM_008957 |
| 75 | NM_138952 |
| 76 | NM_001001321 |
| 77 | NM_010588 |
| 78 | NM_172844 |
| 79 | NM_028450 |
| 80 | NM_030037 |
| 81 | NM_028475 |
| 82 | NM_019773 |
| 83 | NM_178076 |
| 84 | NM_028784 |
| 85 | NM_018852 |
| 86 | NM_007979 |
| 87 | NM_001037755 |
| 88 | NM_173388 |
| 89 | NM_133216 |
| 90 | NM_029947 |
| 91 | NM_001195413 |
| 92 | NM_001110327 |
| 93 | NM_019791 |
| 94 | NM_028175 |
| 95 | NM_028950 |
| 96 | NM_021426 |
| 97 | NM_033524 |
| 98 | NM_013815 |
| 99 | NM_028105 |
| 100 | NM_001014976 |
| 101 | NM_053208 |
| 102 | NM_026209 |
| 103 | NM_001111066 |
| 104 | NM_183315 |
| 105 | NM_134109 |
| 106 | NM_019972 |
| 107 | NM_030696 |
| 108 | NM_178682 |
| 109 | NM_146164 |
| 110 | NM_010484 |
| 111 | NM_010113 |
| 112 | NM_008720 |
| 113 | NM_029094 |
| 114 | NM_029097 |
| 115 | NM_030558 |
| 116 | NM_001039094 |
| 117 | NM_133206 |
| 118 | NM_010442 |
| 119 | NM_001080926 |
| 120 | NM_010402 |
| 121 | NM_010602 |
| 122 | NM_016781 |
| 123 | NM_011454 |
| 124 | NM_021607 |
| 125 | NM_029077 |
| 126 | NM_008770 |
| 127 | NM_021339 |
| 128 | NM_029582 |
| 129 | NM_027328 |
| 130 | NM_010111 |
| 131 | NM_010754 |
| 132 | NM_027838 |
| 133 | NM_028186 |
| 134 | NM_010413 |
| 135 | NM_026769 |
| 136 | NM_027088 |
| 137 | NM_018810 |
| 138 | NM_013880 |
| 139 | NM_008133 |
| 140 | NM_019827 |
| 141 | NM_009929 |
| 142 | NM_028803 |
| 143 | NM_029880 |
| 144 | NM_016792 |
| 145 | NM_010597 |
| 146 | NM_010865 |
| 147 | NM_008860 |
| 148 | XM_006502607 |
| 149 | NM_026536 |
| 150 | NM_145615 |
| 151 | NM_013630 |
| 152 | NM_008797 |
| 153 | NM_008171 |
| 154 | NM_173185 |
| 155 | NM_008842 |
| 156 | NM_134438 |
| 157 | NM_080644 |
| 158 | NM_007513 |
| 159 | NM_009714 |
| 160 | NM_007835 |
| 161 | NM_007659 |
| 162 | NM_145440 |
| 163 | NM_133728 |
| 164 | NM_001113352 |
| 165 | NM_007835 |
| 166 | NM_007878 |
| 167 | NM_007611 |
| 168 | NM_008032 |
| 169 | NM_007659 |
| 170 | NM_146170 |
| 171 | NM_011352 |
| 172 | NM_008772 |
| 173 | NM_007554 |
| 174 | NM_144783 |
| 175 | NM_007931 |
| 176 | NM_008020 |
| 177 | NM_008792 |
| 178 | NM_134160 |
| 179 | NM_134066 |
| 180 | NM_001110204 |
| 181 | NM_008277 |
| 182 | NM_001110208 |
| 183 | NM_008832 |
| 184 | NM_030688 |
| 185 | NM_008426 |
| 186 | NM_030677 |
| 187 | NM_023057 |
| 188 | NM_009993 |
| 189 | NM_172903 |
| 190 | NM_145542 |
| 191 | NM_028882 |
| 192 | NM_026719 |
| 193 | NM_010924 |
| 194 | NM_134028 |
| 195 | NM_054085 |
| 196 | NM_008525 |
| 197 | NM_173071 |
| 198 | NM_029706 |
| 199 | NM_001098170 |
| 200 | NM_178749 |
| 201 | NM_145458 |
| 202 | NM_001105196 |
| 203 | NM_146041 |
| 204 | NM_026482 |
| 205 | NM_145608 |
| 206 | NM_144954 |
| 207 | NM_178907 |
| 208 | NM_172152 |
| 209 | NM_148931 |
| 210 | NM_177185 |
| 211 | NM_175540 |
| 212 | NM_008437 |
| 213 | NM_026406 |
| 214 | NM_146177 |
| 215 | NM_144893 |
| 216 | NM_029294 |
| 217 | NM_178396 |
| 218 | NM_145922 |
| 219 | NM_152804 |
| 220 | NM_212433 |
| 221 | NM_001110497 |
| 222 | NM_021516 |
| 223 | NM_146096 |
| 224 | NM_008174 |
| 225 | NM_011322 |
| 226 | NM_008846 |
| 227 | NM_028850 |
| 228 | NM_173379 |
| 229 | NM_133819 |
| 230 | NM_033573 |
| 231 | NM_139142 |
| 232 | NM_177357 |
| 233 | NM_010957 |
| 234 | NM_009469 |
| 235 | NM_011201 |
| 236 | NM_178746 |
| 237 | NM_008518 |
| 238 | NM_029020 |
| 239 | NM_011846 |
| 240 | NM_144792 |
| 241 | NM_177725 |
| 242 | NM_029005 |
| 243 | NM_198246 |
| 244 | NM_033269 |
| 245 | NM_019513 |
| 246 | NM_148930 |
| 247 | NM_198214 |
| 248 | NM_001029842 |
| 249 | NM_009157 |
| 250 | NM_146003 |
| 251 | NM_029536 |
| 252 | NM_018736 |
| 253 | NM_001081206 |
| 254 | NM_021360 |
| 255 | NM_009370 |
| 256 | NM_013571 |
| 257 | NM_001081306 |
| 258 | NM_144902 |
| 259 | NM_016689 |
| 260 | NM_019719 |
| 261 | NM_019653 |
| 262 | NM_016697 |
| 263 | NM_175025 |
| 264 | NM_016708 |
| 265 | NM_001081315 |
| 266 | NM_013540 |
| 267 | NM_145700 |
| 268 | NM_009097 |
| 269 | NM_145355 |
| 270 | NM_029274 |
| 271 | NM_019802 |
| 272 | NM_153792 |
| 273 | NM_010568 |
| 274 | NM_175087 |
| 275 | NM_199251 |
| 276 | NM_177646 |
| 277 | NM_146086 |
| 278 | NM_146239 |
| 279 | NM_001038701 |
| 280 | NM_144834 |
| 281 | NM_145121 |
| 282 | NM_019827 |
| 283 | NM_177395 |
| 284 | NM_175127 |
| 285 | NM_021390 |
| 286 | NM_138606 |
| 287 | NM_001109045 |
| 288 | NM_016744 |
| 289 | NM_010154 |
| 290 | NM_009595 |
| 291 | NM_021485 |
| 292 | NM_199446 |
| 293 | NM_011083 |
| 294 | NM_001033254 |
| 295 | NM_153135 |
| 296 | NM_011903 |
| 297 | NM_001033209 |
| 298 | NM_213733 |
| 299 | NM_023209 |
| 300 | NM_207683 |
| 301 | NM_010899 |
| 302 | NM_011658 |
| 303 | NM_011716 |
| 304 | NM_010142 |
| 305 | NM_010934 |
| 306 | NM_011815 |
| 307 | NM_018810 |
| 308 | NM_133882 |
| 309 | NM_010043 |
| 310 | NM_001033328 |
| 311 | NM_177648 |
| 312 | NM_010434 |
| 313 | NM_011706 |
| 314 | NM_028779 |
| 315 | NM_175514 |
| 316 | NM_008381 |
| 317 | NM_021565 |
| 318 | NM_010205 |
| 319 | NM_010608 |
| 320 | NM_010728 |
| 321 | NM_133897 |
| 322 | NM_025968 |
| 323 | NM_015826 |
| 324 | NM_009011 |
| 325 | NM_025846 |
| 326 | NM_011218 |
| 327 | NM_175638 |
| 328 | NM_010794 |
| 329 | NM_177992 |
| 330 | NM_175465 |
| 331 | NM_207210 |
| 332 | NM_009809 |
| 333 | NM_001008702 |
| 334 | NM_011846 |
| 335 | NM_177353 |
| 336 | NM_198108 |
| 337 | NM_011077 |
| 338 | NM_021516 |
| 339 | NM_199159 |
| 340 | NM_023383 |
| 341 | NM_001037758 |
| 342 | NM_016975 |
| 343 | NM_213733 |

14. The expression vector of claim 13, wherein the RNAi target gene is a mouse gene or a Chinese hamster ovary (CHO) gene selected from the group of genes listed in the Table immediately below or is an orthologue of the selected mouse or CHO gene.

| Mouse RNAi Target | CHO RNAi Target |
|---|---|
| Gene | Gene |
| (Accession No.) | (Accession No.) |
| NM_001033156 | XM_003511078 |
| NM_028052 | XM_003503668.1 |
| NM_011169 | XM_003504158 |
| NM_008246 | XM_003513214 |
| NM_008957 | XM_003514770 |
| NM_133895 | XM_003505358 |
| NM_010484 | XM_003506082 |
| NM_133206 | XM_003510170 |
| NM_028784 | XM_003497479 |
| NM_010113 | XM_007653160 |
| NM_028950 | XM_003503382 |
| NM_008720 | NM_001246687 |
| NM_133216 | XM_003511942 |
| NM_053208 | XM_003505261 |
| NM_010754 | XM_003501037, |
| | XM_003501038 |
| NM_027838 | XR_135851 |
| NM_028186 | XM_003515516 |
| NM_010413 | XM_003505101 |
| NM_026769 | XM_003512003 |
| NM_008792 | XM_003508113 |
| NM_001110208 | XM_003505253 |
| NM_146170 | XM_003507962, |
| | XM_003507963 |
| NM_008832 | XM_003512672, |
| | XM_003512673 |
| NM_030688 | XM_003507545 |
| NM_080644 | NW_006879584 |
| NM_009993 | XM_003502400 |
| NM_026536 | XM_003511591 |
| NM_001098170 | XM_003495474 |
| NM_010924 | XM_003509317 |
| NM_008437 | XM_003510897 |
| NM_054085 | XM_003511682 |
| NM_177185 | XM_003503221 |
| NM_178396 | XM_003499339 |
| NM_178907 | XM_003500390 |
| NM_145922 | XM_003514579 |
| NM_145458 | XM_003502752 |
| NM_146177 | XM_003508241 |
| NM_144893 | NM_001243991 |
| NM_198246 | XM_003513347 |
| NM_029020 | XM_003501302 |
| NM_033573 | XM_003502900 |
| NM_028850 | XM_003513492 |
| NM_139142 | XM_003507723 |
| NM_011201 | XM_003502483 |
| NM_146096 | XM_003511733 |
| NM_173379 | XM_007642431 |
| NM_177725 | XM_003513970 |
| NM_001081206 | XM_003500967 |
| NM_145355 | XM_003509299 |
| NM_148930 | XM_003500410 |
| NM_013540 | XM_003500211, |
| | XM_003500212 |
| NM_016697 | XM_007643638 |
| NM_146086 | XM_003506313 |
| NM_144834 | XM_003509378 |
| NM_153792 | XM_003501507, |
| | XM_003501508 |
| NM_021485 | XM_003509935 |
| NM_138606 | XM_003505096 |
| NM_011083 | XM_003504810 |
| NM_009595 | XM_003507470, |
| | XM_003507471 |
| NM_177646 | XM_003512693 |
| NM_001109045 | XM_003514321 |
| NM_175514 | XM_003503395 |
| NM_001033328 | XM_007653510 |
| NM_133897 | XM_003506392 |
| NM_025968 | XM_003510575 |
| NM_177648 | XM_003513968 |
| NM_010142 | XM_003503638 |
| NM_010434 | XM_003497435, |
| | XM_003497436 |
| NM_010934 | XM_003510777 |
| NM_011706 | XM_003496342, |
| | XM_003496343 |
| NM_015826 | XM_003514815 |
| NM_001008702 | XM_003503098 |
| NM_016975 | XM_003500430 |
| NM_199159 | XM_003504375 |
| NM_175638 | XM_003504666 |

15. The expression vector of claim 13, wherein the RNAi target gene is a mammalian gene selected from the group of mouse and CHO genes listed in the table immediately below or is an ortholog of the selected mouse or CHO gene.

| Mouse RNAi Target | CHO RNAi Target |
|---|---|
| Gene | Gene |
| (Accession No.) | (Accession No.) |
| NM_009993 | XM_003502400 |
| NM_026536 | XM_003511591 |
| NM_177185 | XM_003503221 |
| NM_173379 | XM_007642431 |
| NM_001081206 | XM_003500967 |
| NM_145355 | XM_003509299 |
| NM_016697 | XM_007643638 |
| NM_153792 | XM_003501507, |
| | XM_003501508 |
| NM_021485 | XM_003509935 |
| NM_199159 | XM_003504375 |
| NM_175638 | XM_003504666 |

16. The expression vector of claim 13, wherein the expression cassette comprises an inducible promoter operably linked to a nucleotide sequence that encodes the shRNA molecule.

17. (canceled)

18. The expression vector of claim 13, wherein the expression cassette comprises SEQ ID NO:499.

19. A recombinant mammalian cell which is stably transfected with the expression vector of claim 13.

20. (canceled)

21. (canceled)

22. A synthetic short-interfering nucleic acid (siNA) molecule for use in inhibiting expression of an RNAi target gene which is a mouse gene selected from the group of mouse genes listed in the table immediately below or is an ortholog of the selected mouse gene.

| Gene | Mouse Gene |
|---|---|
| No. | (Accession No) |
| 1 | NM_024479 |
| 2 | NM_023697 |
| 3 | NM_013523 |
| 4 | NM_029840 |
| 5 | NM_023168 |
| 6 | NM_019980 |
| 7 | NM_013847 |
| 8 | NM_026871 |
| 9 | NM_133355 |
| 10 | NM_001003909 |
| 11 | NM_025350 |
| 12 | NM_025292 |
| 13 | NM_013415 |
| 14 | NM_001081414 |
| 15 | NM_133500 |
| 16 | NM_007958 |
| 17 | NM_021509 |
| 18 | NM_177941 |
| 19 | NM_080510 |
| 20 | NM_001033156 |
| 21 | NM_001081377 |
| 22 | NM_001014995 |
| 23 | NM_023543 |
| 24 | NM_026009 |
| 25 | NM_133684 |
| 26 | NM_021491 |
| 27 | NM_026984 |
| 28 | NM_023131 |
| 29 | NM_025650 |
| 30 | NM_011217 |
| 31 | NM_008048 |
| 32 | NM_025865 |
| 33 | NM_013855 |
| 34 | NM_025964 |
| 35 | NM_001004761 |
| 36 | NM_019828 |
| 37 | NM_011521 |
| 38 | NM_008737 |
| 39 | NM_008770 |
| 40 | NM_001024385 |
| 41 | NM_024267 |
| 42 | NM_008246 |
| 43 | NM_024454 |
| 44 | NM_008626 |
| 45 | NM_007647 |
| 46 | NM_011169 |
| 47 | NM_025485 |
| 48 | NM_199314 |
| 49 | NM_008206 |
| 50 | FJ042496 |
| 51 | NM_028052 |
| 52 | NM_024198 |
| 53 | NM_027880 |
| 54 | NM_028004 |
| 55 | NM_019688 |
| 56 | NM_028006 |
| 57 | NM_027880 |
| 58 | NM_133895 |
| 59 | NM_013563 |
| 60 | NM_013632 |
| 61 | NM_019724 |
| 62 | NM_013659 |
| 63 | NM_177909 |
| 64 | NM_175638 |
| 65 | NM_001081309 |
| 66 | NM_011101 |
| 67 | NM_029572 |
| 68 | NM_183294 |
| 69 | NM_199029 |
| 70 | NM_023517 |
| 71 | NM_024236 |
| 72 | NM_012048 |
| 73 | NM_020603 |
| 74 | NM_008957 |
| 75 | NM_138952 |
| 76 | NM_001001321 |
| 77 | NM_010588 |
| 78 | NM_172844 |
| 79 | NM_028450 |
| 80 | NM_030037 |
| 81 | NM_028475 |
| 82 | NM_019773 |
| 83 | NM_178076 |
| 84 | NM_028784 |
| 85 | NM_018852 |
| 86 | NM_007979 |
| 87 | NM_001037755 |
| 88 | NM_173388 |
| 89 | NM_133216 |
| 90 | NM_029947 |
| 91 | NM_001195413 |
| 92 | NM_001110327 |
| 93 | NM_019791 |
| 94 | NM_028175 |
| 95 | NM_028950 |
| 96 | NM_021426 |
| 97 | NM_033524 |
| 98 | NM_013815 |
| 99 | NM_028105 |
| 100 | NM_001014976 |
| 101 | NM_053208 |
| 102 | NM_026209 |
| 103 | NM_001111066 |
| 104 | NM_183315 |
| 105 | NM_134109 |
| 106 | NM_019972 |
| 107 | NM_030696 |
| 108 | NM_178682 |
| 109 | NM_146164 |
| 110 | NM_010484 |
| 111 | NM_010113 |
| 112 | NM_008720 |
| 113 | NM_029094 |
| 114 | NM_029097 |
| 115 | NM_030558 |
| 116 | NM_001039094 |
| 117 | NM_133206 |
| 118 | NM_010442 |
| 119 | NM_001080926 |
| 120 | NM_010402 |
| 121 | NM_010602 |
| 122 | NM_016781 |
| 123 | NM_011454 |
| 124 | NM_021607 |
| 125 | NM_029077 |
| 126 | NM_008770 |
| 127 | NM_021339 |
| 128 | NM_029582 |
| 129 | NM_027328 |
| 130 | NM_010111 |
| 131 | NM_010754 |
| 132 | NM_027838 |
| 133 | NM_028186 |
| 134 | NM_010413 |
| 135 | NM_026769 |
| 136 | NM_027088 |
| 137 | NM_018810 |
| 138 | NM_013880 |
| 139 | NM_008133 |
| 140 | NM_019827 |
| 141 | NM_009929 |
| 142 | NM_028803 |
| 143 | NM_029880 |
| 144 | NM_016792 |
| 145 | NM_010597 |
| 146 | NM_010865 |
| 147 | NM_008860 |
| 148 | XM_006502607 |
| 149 | NM_026536 |
| 150 | NM_145615 |
| 151 | NM_013630 |
| 152 | NM_008797 |
| 153 | NM_008171 |
| 154 | NM_173185 |
| 155 | NM_008842 |
| 156 | NM_134438 |
| 157 | NM_080644 |
| 158 | NM_007513 |
| 159 | NM_009714 |
| 160 | NM_007835 |
| 161 | NM_007659 |
| 162 | NM_145440 |
| 163 | NM_133728 |
| 164 | NM_001113352 |
| 165 | NM_007835 |
| 166 | NM_007878 |
| 167 | NM_007611 |
| 168 | NM_008032 |
| 169 | NM_007659 |
| 170 | NM_146170 |
| 171 | NM_011352 |
| 172 | NM_008772 |
| 173 | NM_007554 |
| 174 | NM_144783 |
| 175 | NM_007931 |
| 176 | NM_008020 |
| 177 | NM_008792 |
| 178 | NM_134160 |
| 179 | NM_134066 |
| 180 | NM_001110204 |
| 181 | NM_008277 |
| 182 | NM_001110208 |
| 183 | NM_008832 |
| 184 | NM_030688 |
| 185 | NM_008426 |
| 186 | NM_030677 |
| 187 | NM_023057 |
| 188 | NM_009993 |
| 189 | NM_172903 |
| 190 | NM_145542 |
| 191 | NM_028882 |
| 192 | NM_026719 |
| 193 | NM_010924 |
| 194 | NM_134028 |
| 195 | NM_054085 |
| 196 | NM_008525 |
| 197 | NM_173071 |
| 198 | NM_029706 |
| 199 | NM_001098170 |
| 200 | NM_178749 |
| 201 | NM_145458 |
| 202 | NM_001105196 |
| 203 | NM_146041 |
| 204 | NM_026482 |
| 205 | NM_145608 |
| 206 | NM_144954 |
| 207 | NM_178907 |
| 208 | NM_172152 |
| 209 | NM_148931 |
| 210 | NM_177185 |
| 211 | NM_175540 |
| 212 | NM_008437 |
| 213 | NM_026406 |
| 214 | NM_146177 |
| 215 | NM_144893 |
| 216 | NM_029294 |
| 217 | NM_178396 |
| 218 | NM_145922 |
| 219 | NM_152804 |
| 220 | NM_212433 |
| 221 | NM_001110497 |
| 222 | NM_021516 |
| 223 | NM_146096 |
| 224 | NM_008174 |
| 225 | NM_011322 |
| 226 | NM_008846 |
| 227 | NM_028850 |
| 228 | NM_173379 |
| 229 | NM_133819 |
| 230 | NM_033573 |
| 231 | NM_139142 |
| 232 | NM_177357 |
| 233 | NM_010957 |
| 234 | NM_009469 |
| 235 | NM_011201 |
| 236 | NM_178746 |
| 237 | NM_008518 |
| 238 | NM_029020 |
| 239 | NM_011846 |
| 240 | NM_144792 |
| 241 | NM_177725 |
| 242 | NM_029005 |
| 243 | NM_198246 |
| 244 | NM_033269 |
| 245 | NM_019513 |
| 246 | NM_148930 |
| 247 | NM_198214 |
| 248 | NM_001029842 |
| 249 | NM_009157 |
| 250 | NM_146003 |
| 251 | NM_029536 |
| 252 | NM_018736 |
| 253 | NM_001081206 |
| 254 | NM_021360 |
| 255 | NM_009370 |
| 256 | NM_013571 |
| 257 | NM_001081306 |
| 258 | NM_144902 |
| 259 | NM_016689 |
| 260 | NM_019719 |
| 261 | NM_019653 |
| 262 | NM_016697 |
| 263 | NM_175025 |
| 264 | NM_016708 |
| 265 | NM_001081315 |
| 266 | NM_013540 |
| 267 | NM_145700 |
| 268 | NM_009097 |
| 269 | NM_145355 |
| 270 | NM_029274 |
| 271 | NM_019802 |
| 272 | NM_153792 |
| 273 | NM_010568 |
| 274 | NM_175087 |
| 275 | NM_199251 |
| 276 | NM_177646 |
| 277 | NM_146086 |
| 278 | NM_146239 |
| 279 | NM_001038701 |
| 280 | NM_144834 |
| 281 | NM_145121 |
| 282 | NM_019827 |
| 283 | NM_177395 |
| 284 | NM_175127 |
| 285 | NM_021390 |
| 286 | NM_138606 |
| 287 | NM_001109045 |
| 288 | NM_016744 |
| 289 | NM_010154 |
| 290 | NM_009595 |
| 291 | NM_021485 |
| 292 | NM_199446 |
| 293 | NM_011083 |
| 294 | NM_001033254 |
| 295 | NM_153135 |
| 296 | NM_011903 |
| 297 | NM_001033209 |
| 298 | NM_213733 |
| 299 | NM_023209 |
| 300 | NM_207683 |
| 301 | NM_010899 |
| 302 | NM_011658 |
| 303 | NM_011716 |
| 304 | NM_010142 |
| 305 | NM_010934 |
| 306 | NM_011815 |
| 307 | NM_018810 |
| 308 | NM_133882 |
| 309 | NM_010043 |
| 310 | NM_001033328 |
| 311 | NM_177648 |
| 312 | NM_010434 |
| 313 | NM_011706 |
| 314 | NM_028779 |
| 315 | NM_175514 |
| 316 | NM_008381 |
| 317 | NM_021565 |
| 318 | NM_010205 |
| 319 | NM_010608 |
| 320 | NM_010728 |
| 321 | NM_133897 |
| 322 | NM_025968 |
| 323 | NM_015826 |
| 324 | NM_009011 |
| 325 | NM_025846 |
| 326 | NM_011218 |
| 327 | NM_175638 |
| 328 | NM_010794 |
| 329 | NM_177992 |
| 330 | NM_175465 |
| 331 | NM_207210 |
| 332 | NM_009809 |
| 333 | NM_001008702 |
| 334 | NM_011846 |
| 335 | NM_177353 |
| 336 | NM_198108 |
| 337 | NM_011077 |
| 338 | NM_021516 |
| 339 | NM_199159 |
| 340 | NM_023383 |
| 341 | NM_001037758 |
| 342 | NM_016975 |
| 343 | NM_213733 |

23. The siNA of claim 22, wherein the RNAi target gene is selected from the group of genes listed in Table 2 or an ortholog thereof.

24. The siNA of claim 22, wherein the RNAi target gene is wherein the RNAi target gene is a mammalian gene selected from the group of mouse and CHO genes listed in the table immediately below or is an ortholog of the selected mouse or CHO gene.

| Mouse RNAi Target | CHO RNAi Target |
|---|---|
| Gene | Gene |
| (Accession No.) | (Accession No.) |
| NM_009993 | XM_003502400 |
| NM_026536 | XM_003511591 |
| NM_177185 | XM_003503221 |
| NM_173379 | XM_007642431 |
| NM_001081206 | XM_003500967 |
| NM_145355 | XM_003509299 |
| NM_016697 | XM_007643638 |
| NM_153792 | XM_003501507, |
| | XM_003501508 |
| NM_021485 | XM_003509935 |
| NM_199159 | XM_003504375 |
| NM_175638 | XM_003504666 |

25. The siNA of claim 22, wherein the siNA molecule is an siRNA which comprises
(a) an antisense strand comprising a first nucleotide sequence of at least 15 nucleotides that is complementary to at least 15 contiguous nucleotides of an RNAi target sequence selected group of sequences consisting of SEQ ID NOs:1-343; and
(b) a sense strand comprising a second nucleotide sequence of at least 15 nucleotides that is complementary to the first nucleotide sequence.

26. The siNA of claim 25, wherein the RNAi target sequence is selected from the group of sequences consisting of SEQ ID NO:188, SEQ ID NO:149, SEQ ID NO:210, SEQ ID NO:228, SEQ ID NO:253, SEQ ID NO:269, SEQ ID NO:262, SEQ ID NO:272, SEQ ID NO:291, SEQ ID NO:339 and SEQ ID NO:32.

27. The siNA of claim 25, wherein the first and second nucleotide sequences are a pair of antisense and sense sequences listed in the table immediately below for SEQ ID NO:188, SEQ ID NO:149, SEQ ID NO:210, SEQ ID NO:228, SEQ ID NO:253, SEQ ID NO:269, SEQ ID NO:262, SEQ ID NO:272, SEQ ID NO:291, SEQ ID NO:339 or SEQ ID NO:327.

| RNAi | | |
|---|---|---|
| Target | | |
| Se- | | |
| quence | siRNA | |
| ID NO. | ANTISENSE SEQUENCE | SENSE SEQUENCE |
| 188 | AUGUGUAUCGGUAGAUCUCTT | GAGAUCUACCGAUACACAUTT |
| | (SEQ ID NO: 394) | (SEQ ID NO: 395) |
| 149 | UCCAUGAUACAAGAAUCGGTT | CCGAUUCUUGUAUCAUGGATT |
| | (SEQ ID NO: 396) | (SEQ ID NO: 397) |
| 210 | UACACGAAGGUCCUCAAUGTT | CAUUGAGGACCUUCGUGUATT |
| | (SEQ ID NO: 406) | (SEQ ID NO: 407) |
| 228 | UGACGUUUGACAAAUGCUGTT | CAGCAUUUGUCAAACGUCATT |
| | (SEQ ID NO: 434) | (SEQ ID NO: 435) |
| 253 | UUCAUCGUGAGAAAUCUCCTT | GGAGAUUUCUCACGAUGAATT |
| | (SEQ ID NO: 438) | (SEQ ID NO: 439) |
| 269 | AAUGCUGUGGCAAAUAUCCTT | GGAUAUUUGCCACAGCAUUTT |
| | (SEQ ID NO: 440) | (SEQ ID NO: 441) |
| 262 | UAAGCAGAUCUAUAUUGGCTT | GCCAAUAUAGAUCUGCUUATT |
| | (SEQ ID NO: 446) | (SEQ ID NO: 447) |
| 272 | UUGAUUGUCUGGUAAGAGCTT | GCUCUUACCAGACAAUCAATT |
| | (SEQ ID NO: 452) | (SEQ ID NO: 453) |
| 291 | ACAGAUUCUAGAAUGUUCCTT | GGAACAUUCUAGAAUCUGUTT |
| | (SEQ ID NO: 454) | (SEQ ID NO: 455) |
| 339 | AAUAUUCUUACCUAUCACCTT | GGUGAUAGGUAAGAAUAUUTT |
| | (SEQ ID NO: 490) | (SEQ ID NO: 491) |
| 327 | UCCAAUCUCAAUGUCAAACTT | GUUUGACAUUGAGAUUGGATT |
| | (SEQ ID NO: 492) | (SEQ ID NO: 493) |


SEQUENCE LISTING (499 sequences)

SEQ ID NO: 1 - DNA, 19 bp, Artificial Sequence
  RNAI TARGET SEQUENCE
cctgcaagctccgcttcta

SEQ ID NO: 2 - DNA, 19 bp, Artificial Sequence
  RNAI TARGET SEQUENCE
ctttgaagacttgaacagt

SEQ ID NO: 3 - DNA, 19 bp, Artificial Sequence
  RNAI TARGET SEQUENCE
ccaagattgccaagcgcat

SEQ ID NO: 4 - DNA, 19 bp, Artificial Sequence
  RNAI TARGET SEQUENCE
cataccattggatgaggta

SEQ ID NO: 5 - DNA, 19 bp, Artificial Sequence
  RNAI TARGET SEQUENCE
ctctgcatcttcatccgga

SEQ ID NO: 6 - DNA, 19 bp, Artificial Sequence
  RNAI TARGET SEQUENCE
cctacaagcgcttgtagga

SEQ ID NO: 7 - DNA, 19 bp, Artificial Sequence
  RNAI TARGET SEQUENCE
ctcaacttctgtgccaata

SEQ ID NO: 8 - DNA, 19 bp, Artificial Sequence
  RNAI TARGET SEQUENCE
gaagcccattcctcgaatt

SEQ ID NO: 9 - DNA, 19 bp, Artificial Sequence
  RNAI TARGET SEQUENCE
gagctaaactccaccaaga

SEQ ID NO: 10 - DNA, 19 bp, Artificial Sequence
  RNAI TARGET SEQUENCE
cagataccactttcatcga

SEQ ID NO: 11 - DNA, 19 bp, Artificial Sequence
  RNAI TARGET SEQUENCE
caagtatggcagtatcatt

SEQ ID NO: 12 - DNA, 19 bp, Artificial Sequence
  RNAI TARGET SEQUENCE
cagtatgtctccaacgaca

SEQ ID NO: 13 - DNA, 19 bp, Artificial Sequence
  RNAI TARGET SEQUENCE
gcgatattatgagcaacct

SEQ ID NO: 14 - DNA, 19 bp, Artificial Sequence
  RNAI TARGET SEQUENCE
ctacacatcttctggcaga

SEQ ID NO: 15 - DNA, 19 bp, Artificial Sequence
  RNAI TARGET SEQUENCE
gcatggaggccttcggcat

SEQ ID NO: 16 - DNA, 19 bp, Artificial Sequence
  RNAI TARGET SEQUENCE
gacattgattgcaatccat

SEQ ID NO: 17 - DNA, 19 bp, Artificial Sequence
  RNAI TARGET SEQUENCE
ctcataccttctttattat

SEQ ID NO: 18 - DNA, 19 bp, Artificial Sequence
  RNAI TARGET SEQUENCE
gaatgtatcttctgaaggt

SEQ ID NO: 19 - DNA, 19 bp, Artificial Sequence
  RNAI TARGET SEQUENCE
cattcaggcacggatggaa

SEQ ID NO: 20 - DNA, 19 bp, Artificial Sequence
  RNAI TARGET SEQUENCE
gaactagagcggtttgtaa

SEQ ID NO: 21 - DNA, 19 bp, Artificial Sequence
  RNAI TARGET SEQUENCE
gtaattgagctgtcagttt

SEQ ID NO: 22 - DNA, 19 bp, Artificial Sequence
  RNAI TARGET SEQUENCE
ccatttgtgctgcaataat

SEQ ID NO: 23 - DNA, 19 bp, Artificial Sequence
  RNAI TARGET SEQUENCE
ggacatatgtattcgggaa

SEQ ID NO: 24 - DNA, 19 bp, Artificial Sequence
  RNAI TARGET SEQUENCE
catagtagagtatgatgat

SEQ ID NO: 25 - DNA, 19 bp, Artificial Sequence
  RNAI TARGET SEQUENCE
gaaactttacccttctgaa

SEQ ID NO: 26 - DNA, 19 bp, Artificial Sequence
  RNAI TARGET SEQUENCE
ctctaagggagctctgttt

SEQ ID NO: 27 - DNA, 19 bp, Artificial Sequence
  RNAI TARGET SEQUENCE
gtgataccagtgcaactga

SEQ ID NO: 28 - DNA, 19 bp, Artificial Sequence
  RNAI TARGET SEQUENCE
gacatgcgctggctacgaa

SEQ ID NO: 29 - DNA, 19 bp, Artificial Sequence
  RNAI TARGET SEQUENCE
ggactgggtgccttacatc

SEQ ID NO: 30 - DNA, 19 bp, Artificial Sequence
  RNAI TARGET SEQUENCE
cacagtttggaattacaga

SEQ ID NO: 31 - DNA, 19 bp, Artificial Sequence
  RNAI TARGET SEQUENCE
cttgcctggtgaccgggaa

SEQ ID NO: 32 - DNA, 19 bp, Artificial Sequence
  RNAI TARGET SEQUENCE
gcattatgcggacattcat

SEQ ID NO: 33 - DNA, 19 bp, Artificial Sequence
  RNAI TARGET SEQUENCE
cctagaagatgagcaccaa

SEQ ID NO: 34 - DNA, 19 bp, Artificial Sequence
  RNAI TARGET SEQUENCE
ccagaaattccataagcct

SEQ ID NO: 35 - DNA, 19 bp, Artificial Sequence
  RNAI TARGET SEQUENCE
cattgaccaatgttcgagt

SEQ ID NO: 36 - DNA, 19 bp, Artificial Sequence
  RNAI TARGET SEQUENCE
gaaaccaggtccacaggat

SEQ ID NO: 37 - DNA, 19 bp, Artificial Sequence
  RNAI TARGET SEQUENCE
gcagttacgacttgggcaa

SEQ ID NO: 38 - DNA, 19 bp, Artificial Sequence
  RNAI TARGET SEQUENCE
cactgtatgaccttctggt

SEQ ID NO: 39 - DNA, 19 bp, Artificial Sequence
  RNAI TARGET SEQUENCE
cattccattgttgttgatt

SEQ ID NO: 40 - DNA, 19 bp, Artificial Sequence
  RNAI TARGET SEQUENCE
cattcatcagcaaggtaaa

SEQ ID NO: 41 - DNA, 19 bp, Artificial Sequence
  RNAI TARGET SEQUENCE
cagagtatgacgccatgtt

SEQ ID NO: 42 - DNA, 19 bp, Artificial Sequence
  RNAI TARGET SEQUENCE
ggatcaacagggtgttgtt

SEQ ID NO: 43 - DNA, 19 bp, Artificial Sequence
  RNAI TARGET SEQUENCE
caattacgtttataacact

SEQ ID NO: 44 - DNA, 19 bp, Artificial Sequence
  RNAI TARGET SEQUENCE
cagtttaacttccaatcca

SEQ ID NO: 45 - DNA, 19 bp, Artificial Sequence
  RNAI TARGET SEQUENCE
gtttcgaagtgcctgttta

SEQ ID NO: 46 - DNA, 19 bp, Artificial Sequence
  RNAI TARGET SEQUENCE
gacttatatccaggacaaa

SEQ ID NO: 47 - DNA, 19 bp, Artificial Sequence
  RNAI TARGET SEQUENCE
gccaataaatgacgtatta

SEQ ID NO: 48 - DNA, 19 bp, Artificial Sequence
  RNAI TARGET SEQUENCE
ctcatggttctcttgaact

SEQ ID NO: 49 - DNA, 19 bp, Artificial Sequence
  RNAI TARGET SEQUENCE
ctaaccacaggttccggaa

SEQ ID NO: 50 - DNA, 19 bp, Artificial Sequence
  RNAI TARGET SEQUENCE
ccttatggtgtcttgcaat

SEQ ID NO: 51 - DNA, 19 bp, Artificial Sequence
  RNAI TARGET SEQUENCE
cttcctttaacaatcagat

SEQ ID NO: 52 - DNA, 19 bp, Artificial Sequence
  RNAI TARGET SEQUENCE
cttgcaaccagtttggcca

SEQ ID NO: 53 - DNA, 19 bp, Artificial Sequence
  RNAI TARGET SEQUENCE
gcatgattgtggccgaggt

SEQ ID NO: 54 - DNA, 19 bp, Artificial Sequence
  RNAI TARGET SEQUENCE
cagataaccaccacgatgt

SEQ ID NO: 55 - DNA, 19 bp, Artificial Sequence
  RNAI TARGET SEQUENCE
gaaggtacctcctggtaca

SEQ ID NO: 56 - DNA, 19 bp, Artificial Sequence
  RNAI TARGET SEQUENCE
cacatttctgttaaaggtt

SEQ ID NO: 57 - DNA, 19 bp, Artificial Sequence
  RNAI TARGET SEQUENCE
gagatgatcagttggtact

SEQ ID NO: 58 - DNA, 19 bp, Artificial Sequence
  RNAI TARGET SEQUENCE
caggaatcctggagagtaa

SEQ ID NO: 59 - DNA, 19 bp, Artificial Sequence
  RNAI TARGET SEQUENCE
gaatctagaggatctggtt

SEQ ID NO: 60 - DNA, 19 bp, Artificial Sequence
  RNAI TARGET SEQUENCE
gaacaaggttgtcatggat

SEQ ID NO: 61 - DNA, 19 bp, Artificial Sequence
  RNAI TARGET SEQUENCE
cagtattgatgcagtttat

SEQ ID NO: 62 - DNA, 19 bp, Artificial Sequence
  RNAI TARGET SEQUENCE
gctacaagaccctgccttt

SEQ ID NO: 63 - DNA, 19 bp, Artificial Sequence
  RNAI TARGET SEQUENCE
catactctatatccatcta

SEQ ID NO: 64 - DNA, 19 bp, Artificial Sequence
  RNAI TARGET SEQUENCE
cggataagaacgagaggtt

SEQ ID NO: 65 - DNA, 19 bp, Artificial Sequence
  RNAI TARGET SEQUENCE
cggatttcaactatttctt

SEQ ID NO: 66 - DNA, 19 bp, Artificial Sequence
  RNAI TARGET SEQUENCE
caaccaagaagagggcgaa

SEQ ID NO: 67 - DNA, 19 bp, Artificial Sequence
  RNAI TARGET SEQUENCE
cttacaactggattcaaga

SEQ ID NO: 68 - DNA, 19 bp, Artificial Sequence
  RNAI TARGET SEQUENCE
gcaagtatttagcatgaat

SEQ ID NO: 69 - DNA, 19 bp, Artificial Sequence
  RNAI TARGET SEQUENCE
gccgtaaggtgtacggcat

SEQ ID NO: 70 - DNA, 19 bp, Artificial Sequence
  RNAI TARGET SEQUENCE
gtgcctacaatagctgcta

SEQ ID NO: 71 - DNA, 19 bp, Artificial Sequence
  RNAI TARGET SEQUENCE
gaaatcaatgcctgaggca

SEQ ID NO: 72 - DNA, 19 bp, Artificial Sequence
  RNAI TARGET SEQUENCE
ctcataagaagagtttctt

SEQ ID NO: 73 - DNA, 19 bp, Artificial Sequence
  RNAI TARGET SEQUENCE
gcactttgacctgaatcta

SEQ ID NO: 74 - DNA, 19 bp, Artificial Sequence
  RNAI TARGET SEQUENCE
ggtttatccaggaagtata

SEQ ID NO: 75 - DNA, 19 bp, Artificial Sequence
  RNAI TARGET SEQUENCE
gttatcatgtgggaagtgt

SEQ ID NO: 76 - DNA, 19 bp, Artificial Sequence
  RNAI TARGET SEQUENCE
ggagtttataccaaacaga

SEQ ID NO: 77 - DNA, 19 bp, Artificial Sequence
  RNAI TARGET SEQUENCE
caagttctgtgacgagtgt

SEQ ID NO: 78 - DNA, 19 bp, Artificial Sequence
  RNAI TARGET SEQUENCE
gaagtttgaaggctgttat

SEQ ID NO: 79 - DNA, 19 bp, Artificial Sequence
  RNAI TARGET SEQUENCE
aggatattcactttcatat

SEQ ID NO: 80 - DNA, 19 bp, Artificial Sequence
  RNAI TARGET SEQUENCE
gacatcacctctgttcttc

SEQ ID NO: 81 - DNA, 19 bp, Artificial Sequence
  RNAI TARGET SEQUENCE
gatgtcaacagagccatca

SEQ ID NO: 82 - DNA, 19 bp, Artificial Sequence
  RNAI TARGET SEQUENCE
ctgttgcctgcttacattt

SEQ ID NO: 83 - DNA, 19 bp, Artificial Sequence
  RNAI TARGET SEQUENCE
gctacaagcagtccttgaa

SEQ ID NO: 84 - DNA, 19 bp, Artificial Sequence
  RNAI TARGET SEQUENCE
ggatgatgaaggtgttctt

SEQ ID NO: 85 - DNA, 19 bp, Artificial Sequence
  RNAI TARGET SEQUENCE
caataaagaagatatagtt

SEQ ID NO: 86 - DNA, 19 bp, Artificial Sequence
  RNAI TARGET SEQUENCE
ctgtgttgccaatagggaa

SEQ ID NO: 87 - DNA, 19 bp, Artificial Sequence
  RNAI TARGET SEQUENCE
gaatgtacctgtcatgaat

SEQ ID NO: 88 - DNA, 19 bp, Artificial Sequence
  RNAI TARGET SEQUENCE
cctctatacctccatcttt

SEQ ID NO: 89 - DNA, 19 bp, Artificial Sequence
  RNAI TARGET SEQUENCE
gattgcatggctgttcaat

SEQ ID NO: 90 - DNA, 19 bp, Artificial Sequence
  RNAI TARGET SEQUENCE
gaatctcgaagcttatata

SEQ ID NO: 91 - DNA, 19 bp, Artificial Sequence
  RNAI TARGET SEQUENCE
gtcaagacctggtatgagt

SEQ ID NO: 92 - DNA, 19 bp, Artificial Sequence
  RNAI TARGET SEQUENCE
cagatcattgttcatgctt

SEQ ID NO: 93 - DNA, 19 bp, Artificial Sequence
  RNAI TARGET SEQUENCE
gagatgtggcccttcttca

SEQ ID NO: 94 - DNA, 19 bp, Artificial Sequence
  RNAI TARGET SEQUENCE
gctcaaagtgtgtaagttt

SEQ ID NO: 95 - DNA, 19 bp, Artificial Sequence
  RNAI TARGET SEQUENCE
ccaaatatggcttgtactt

SEQ ID NO: 96 - DNA, 19 bp, Artificial Sequence
  RNAI TARGET SEQUENCE
cctggaatgtcttcattat

SEQ ID NO: 97 - DNA, 19 bp, Artificial Sequence
  RNAI TARGET SEQUENCE
gacattcagagccgaagta

SEQ ID NO: 98 - DNA, 19 bp, Artificial Sequence
  RNAI TARGET SEQUENCE
gtgtttgtggaactgctta

SEQ ID NO: 99 - DNA, 19 bp, Artificial Sequence
  RNAI TARGET SEQUENCE
ctcaacatgtctcggtgtt

SEQ ID NO: 100 - DNA, 19 bp, Artificial Sequence
  RNAI TARGET SEQUENCE
cttggtacttgctacgtgt

SEQ ID NO: 101 - DNA, 19 bp, Artificial Sequence
  RNAI TARGET SEQUENCE
cagctagcatcgggacaga

SEQ ID NO: 102 - DNA, 19 bp, Artificial Sequence
  RNAI TARGET SEQUENCE
catgttctattggatgtat

SEQ ID NO: 103 - DNA, 19 bp, Artificial Sequence
  RNAI TARGET SEQUENCE
gcactgttccgcaagggca

SEQ ID NO: 104 - DNA, 19 bp, Artificial Sequence
  RNAI TARGET SEQUENCE
ggcagttcgactatgcgtt

SEQ ID NO: 105 - DNA, 19 bp, Artificial Sequence
  RNAI TARGET SEQUENCE
ccaagtttcatcttatgca

SEQ ID NO: 106 - DNA, 19 bp, Artificial Sequence
  RNAI TARGET SEQUENCE
gaactttaaggatattaca

SEQ ID NO: 107 - DNA, 19 bp, Artificial Sequence
  RNAI TARGET SEQUENCE
gaagcattatccagatcta

SEQ ID NO: 108 - DNA, 19 bp, Artificial Sequence
  RNAI TARGET SEQUENCE
ctgctcttctgcaggtgga

SEQ ID NO: 109 - DNA, 19 bp, Artificial Sequence
  RNAI TARGET SEQUENCE
gaaatgagtccacagatga

SEQ ID NO: 110 - DNA, 19 bp, Artificial Sequence
  RNAI TARGET SEQUENCE
ggagtatcatcttgggcta

SEQ ID NO: 111 - DNA, 19 bp, Artificial Sequence
  RNAI TARGET SEQUENCE
cgatttggatagccaacaa

SEQ ID NO: 112 - DNA, 19 bp, Artificial Sequence
  RNAI TARGET SEQUENCE
ccacttacttcatgaccta

SEQ ID NO: 113 - DNA, 19 bp, Artificial Sequence
  RNAI TARGET SEQUENCE
cactaattcagttccagta

SEQ ID NO: 114 - DNA, 19 bp, Artificial Sequence
  RNAI TARGET SEQUENCE
gacagtacccagcgacttt

SEQ ID NO: 115 - DNA, 19 bp, Artificial Sequence
  RNAI TARGET SEQUENCE
cacagtgctacttcgagta

SEQ ID NO: 116 - DNA, 19 bp, Artificial Sequence
  RNAI TARGET SEQUENCE
caaggaattatcattcaga

SEQ ID NO: 117 - DNA, 19 bp, Artificial Sequence
  RNAI TARGET SEQUENCE
gtgagtgtgtgatctgcct

SEQ ID NO: 118 - DNA, 19 bp, Artificial Sequence
  RNAI TARGET SEQUENCE
gcaacagtggcagtgggaa

SEQ ID NO: 119 - DNA, 19 bp, Artificial Sequence
  RNAI TARGET SEQUENCE
gaattacaagggctacttt

SEQ ID NO: 120 - DNA, 19 bp, Artificial Sequence
  RNAI TARGET SEQUENCE
gccttcaaggcggagatca

SEQ ID NO: 121 - DNA, 19 bp, Artificial Sequence
  RNAI TARGET SEQUENCE
ctacctagctgacgagatt

SEQ ID NO: 122 - DNA, 19 bp, Artificial Sequence
  RNAI TARGET SEQUENCE
ggaattcatgtctaagtct

SEQ ID NO: 123 - DNA, 19 bp, Artificial Sequence
  RNAI TARGET SEQUENCE
gaaatgcctttcaatgtca

SEQ ID NO: 124 - DNA, 19 bp, Artificial Sequence
  RNAI TARGET SEQUENCE
ctctatgggttcctggtta

SEQ ID NO: 125 - DNA, 19 bp, Artificial Sequence
  RNAI TARGET SEQUENCE
cagattactcctggatgaa

SEQ ID NO: 126 - DNA, 19 bp, Artificial Sequence
  RNAI TARGET SEQUENCE
gcattccattgttgttgat

SEQ ID NO: 127 - DNA, 19 bp, Artificial Sequence
  RNAI TARGET SEQUENCE
gttatggtcctcattctta

SEQ ID NO: 128 - DNA, 19 bp, Artificial Sequence
  RNAI TARGET SEQUENCE
ccacaattgttgatgtgaa

SEQ ID NO: 129 - DNA, 19 bp, Artificial Sequence
  RNAI TARGET SEQUENCE
gatgaagattgaggagtat

SEQ ID NO: 130 - DNA, 19 bp, Artificial Sequence
  RNAI TARGET SEQUENCE
gacaaattggatattattt

SEQ ID NO: 131 - DNA, 19 bp, Artificial Sequence
  RNAI TARGET SEQUENCE
gaattgagccacagagtaa

SEQ ID NO: 132 - DNA, 19 bp, Artificial Sequence
  RNAI TARGET SEQUENCE
gaaactgaaggctttctta

SEQ ID NO: 133 - DNA, 19 bp, Artificial Sequence
  RNAI TARGET SEQUENCE
gaactacacatctaagttt

SEQ ID NO: 134 - DNA, 19 bp, Artificial Sequence
  RNAI TARGET SEQUENCE
cacagttcatcttcgacca

SEQ ID NO: 135 - DNA, 19 bp, Artificial Sequence
  RNAI TARGET SEQUENCE
caaagaagtttgcagattt

SEQ ID NO: 136 - DNA, 19 bp, Artificial Sequence
  RNAI TARGET SEQUENCE
cgaatgaaggatttcacca

SEQ ID NO: 137 - DNA, 19 bp, Artificial Sequence
  RNAI TARGET SEQUENCE
cctttgagctgggcgagat

SEQ ID NO: 138 - DNA, 19 bp, Artificial Sequence
  RNAI TARGET SEQUENCE
ctttacctggaatattaca

SEQ ID NO: 139 - DNA, 19 bp, Artificial Sequence
  RNAI TARGET SEQUENCE
gttcttgtgactcattagt

SEQ ID NO: 140 - DNA, 19 bp, Artificial Sequence
  RNAI TARGET SEQUENCE
cacagaacctcttgttgga

SEQ ID NO: 141 - DNA, 19 bp, Artificial Sequence
  RNAI TARGET SEQUENCE
gttagaaatggcttccgga

SEQ ID NO: 142 - DNA, 19 bp, Artificial Sequence
  RNAI TARGET SEQUENCE
gtcgttatggaactcctga

SEQ ID NO: 143 - DNA, 19 bp, Artificial Sequence
  RNAI TARGET SEQUENCE
ggatctttggctgggcaga

SEQ ID NO: 144 - DNA, 19 bp, Artificial Sequence
  RNAI TARGET SEQUENCE
ctttgacaactgtttacga

SEQ ID NO: 145 - DNA, 19 bp, Artificial Sequence
  RNAI TARGET SEQUENCE
ggagttaatctcttcgaca

SEQ ID NO: 146 - DNA, 19 bp, Artificial Sequence
  RNAI TARGET SEQUENCE
ccatagtcctctccaaatt

SEQ ID NO: 147 - DNA, 19 bp, Artificial Sequence
  RNAI TARGET SEQUENCE
gaatgaccctgtcacttta

SEQ ID NO: 148 - DNA, 19 bp, Artificial Sequence
  RNAI TARGET SEQUENCE
ggtactttcagtttccaga

SEQ ID NO: 149 - DNA, 19 bp, Artificial Sequence
  RNAI TARGET SEQUENCE
ccgattcttgtatcatgga

SEQ ID NO: 150 - DNA, 19 bp, Artificial Sequence
  RNAI TARGET SEQUENCE
gcaagttggtcagacagga

SEQ ID NO: 151 - DNA, 19 bp, Artificial Sequence
  RNAI TARGET SEQUENCE
gacgtatggctcaaatggt

SEQ ID NO: 152 - DNA, 19 bp, Artificial Sequence
  RNAI TARGET SEQUENCE
cactacttcatcgaggtca

SEQ ID NO: 153 - DNA, 19 bp, Artificial Sequence
  RNAI TARGET SEQUENCE
ctaacatgctgaataggta

SEQ ID NO: 154 - DNA, 19 bp, Artificial Sequence
  RNAI TARGET SEQUENCE
cagatctgtttgaacggaa

SEQ ID NO: 155 - DNA, 19 bp, Artificial Sequence
  RNAI TARGET SEQUENCE
ccaagtgttcttcaggcaa

SEQ ID NO: 156 - DNA, 19 bp, Artificial Sequence
  RNAI TARGET SEQUENCE
gtcattagagttgcccaga

SEQ ID NO: 157 - DNA, 18 bp, Artificial Sequence
  RNAI TARGET SEQUENCE
tctagagtgccaatcata

SEQ ID NO: 158 - DNA, 19 bp, Artificial Sequence
  RNAI TARGET SEQUENCE
ctcatttaaggtacccttt

SEQ ID NO: 159 - DNA, 19 bp, Artificial Sequence
  RNAI TARGET SEQUENCE
cacttggattggcctaact

SEQ ID NO: 160 - DNA, 19 bp, Artificial Sequence
  RNAI TARGET SEQUENCE
cacatcaagttcacccaga

SEQ ID NO: 161 - DNA, 19 bp, Artificial Sequence
  RNAI TARGET SEQUENCE
catggattcttcactcgtt

SEQ ID NO: 162 - DNA, 19 bp, Artificial Sequence
  RMAI TARGET SEQUENCE
gtcattgggctgctctata

SEQ ID NO: 163 - DNA, 19 bp, Artificial Sequence
  RNAI TARGET SEQUENCE
gcaatgtgttcttatggaa

SEQ ID NO: 164 - DNA, 19 bp, Artificial Sequence
  RNAI TARGET SEQUENCE
gaattatgggacgattatt

SEQ ID NO: 165 - DNA, 19 bp, Artificial Sequence
  RNAI TARGET SEQUENCE
ggaagtatttcacatgtga

SEQ ID NO: 166 - DNA, 19 bp, Artificial Sequence
  RNAI TARGET SEQUENCE
gagagtcctgccggtggta

SEQ ID NO: 167 - DNA, 19 bp, Artificial Sequence
  RNAI TARGET SEQUENCE
caatgactgctcttgtgca

SEQ ID NO: 168 - DNA, 19 bp, Artificial Sequence
  RNAI TARGET SEQUENCE
catgctatgaagtactcca

SEQ ID NO: 169 - DNA, 19 bp, Artificial Sequence
  RNAI TARGET SEQUENCE
ctatgatcctgccaaacga

SEQ ID NO: 170 - DNA, 19 bp, Artificial Sequence
  RNAI TARGET SEQUENCE
ccctttatcattgagtgga

SEQ ID NO: 171 - DNA, 19 bp, Artificial Sequence
  RNAI TARGET SEQUENCE
cacagaaaccttccaggta

SEQ ID NO: 172 - DNA, 19 bp, Artificial Sequence
  RNAI TARGET SEQUENCE
gctgttatggattaattgt

SEQ ID NO: 173 - DNA, 19 bp, Artificial Sequence
  RNAI TARGET SEQUENCE
gaggagtttccatcacgaa

SEQ ID NO: 174 - DNA, 19 bp, Artificial Sequence
  RNAI TARGET SEQUENCE
gagaaacgtcctttcatgt

SEQ ID NO: 175 - DNA, 19 bp, Artificial Sequence
  RNAI TARGET SEQUENCE
cacacttcttcaaggtgct

SEQ ID NO: 176 - DNA, 19 bp, Artificial Sequence
  RNAI TARGET SEQUENCE
gctagaagatgggacggag

SEQ ID NO: 177 - DNA, 19 bp, Artificial Sequence
  RNAI TARGET SEQUENCE
caagaaggatttgaccgta

SEQ ID NO: 178 - DNA, 19 bp, Artificial Sequence
  RNAI TARGET SEQUENCE
cagatcatctttgcagtga

SEQ ID NO: 179 - DNA, 19 bp, Artificial Sequence
  RNAI TARGET SEQUENCE
ctcattcatttcccagtgt

SEQ ID NO: 180 - DNA, 19 bp, Artificial Sequence
  RNAI TARGET SEQUENCE
catccaagtggattgcttt

SEQ ID NO: 181 - DNA, 19 bp, Artificial Sequence
  RNAI TARGET SEQUENCE
ctacactggccgtttctta

SEQ ID NO: 182 - DNA, 19 bp, Artificial Sequence
  RNAI TARGET SEQUENCE
ctatccagatggtcgccaa

SEQ ID NO: 183 - DNA, 19 bp, Artificial Sequence
  RNAI TARGET SEQUENCE
ctgctatttccatccatga

SEQ ID NO: 184 - DNA, 19 bp, Artificial Sequence
  RNAI TARGET SEQUENCE
cctataccttcaattgcca

SEQ ID NO: 185 - DNA, 19 bp, Artificial Sequence
  RNAI TARGET SEQUENCE
cgatttgccaccaaagctt

SEQ ID NO: 186 - DNA, 19 bp, Artificial Sequence
  RNAI TARGET SEQUENCE
catcgagcctgacatcaaa

SEQ ID NO: 187 - DNA, 19 bp, Artificial Sequence
  RNAI TARGET SEQUENCE
ccaactatggcatcgtcac

SEQ ID NO: 188 - DNA, 19 bp, Artificial Sequence
  RNAI TARGET SEQUENCE
gagatctaccgatacacat

SEQ ID NO: 189 - DNA, 19 bp, Artificial Sequence
  RNAI TARGET SEQUENCE
gttcgaaggctggtgggaa

SEQ ID NO: 190 - DNA, 19 bp, Artificial Sequence
  RNAI TARGET SEQUENCE
gcattgaccgctgtgtcaa

SEQ ID NO: 191 - DNA, 19 bp, Artificial Sequence
  RNAI TARGET SEQUENCE
ccaacaaggaggaactcat

SEQ ID NO: 192 - DNA, 19 bp, Artificial Sequence
  RNAI TARGET SEQUENCE
cagaattacttaatagaga

SEQ ID NO: 193 - DNA, 19 bp, Artificial Sequence
  RNAI TARGET SEQUENCE
ctatgtgtgtgatcttgaa

SEQ ID NO: 194 - DNA, 19 bp, Artificial Sequence
  RNAI TARGET SEQUENCE
gaaagttcctgccagcagt

SEQ ID NO: 195 - DNA, 19 bp, Artificial Sequence
  RNAI TARGET SEQUENCE
ctaccaaacttcgaggata

SEQ ID NO: 196 - DNA, 19 bp, Artificial Sequence
  RNAI TARGET SEQUENCE
gagctgacatgctcatggt

SEQ ID NO: 197 - DNA, 19 bp, Artificial Sequence
  RNAI TARGET SEQUENCE
ccttcaagagggccaccta

SEQ ID NO: 198 - DNA, 19 bp, Artificial Sequence
  RNAI TARGET SEQUENCE
gtattgataagcaacgtga

SEQ ID NO: 199 - DNA, 19 bp, Artificial Sequence
  RNAI TARGET SEQUENCE
cttatgagctggtgatcga

SEQ ID NO: 200 - DNA, 19 bp, Artificial Sequence
  RNAI TARGET SEQUENCE
gcaattgtaacttaccctt

SEQ ID NO: 201 - DNA, 19 bp, Artificial Sequence
  RNAI TARGET SEQUENCE
gccattattcagtgatgtt

SEQ ID NO: 202 - DNA, 19 bp, Artificial Sequence
  RNAI TARGET SEQUENCE
cgattcaacattaacgata

SEQ ID NO: 203 - DNA, 19 bp, Artificial Sequence
  RNAI TARGET SEQUENCE
gtcgaattgaacatttata

SEQ ID NO: 204 - DNA, 19 bp, Artificial Sequence
  RNAI TARGET SEQUENCE
gataccttctgggttcaga

SEQ ID NO: 205 - DNA, 19 bp, Artificial Sequence
  RNAI TARGET SEQUENCE
ctcgtttgccttaattctt

SEQ ID NO: 206 - DNA, 19 bp, Artificial Sequence
  RNAI TARGET SEQUENCE
gagagaaacttgatgggaa

SEQ ID NO: 207 - DNA, 19 bp, Artificial Sequence
  RNAI TARGET SEQUENCE
ctaacaaccggctcctcaa

SEQ ID NO: 208 - DNA, 19 bp, Artificial Sequence
  RNAI TARGET SEQUENCE
gtctctaacaccatcggaa

SEQ ID NO: 209 - DNA, 19 bp, Artificial Sequence
  RNAI TARGET SEQUENCE
gagatgatgattggattca

SEQ ID NO: 210 - DNA, 19 bp, Artificial Sequence
  RNAI TARGET SEQUENCE
cattgaggaccttcgtgta

SEQ ID NO: 211 - DNA, 19 bp, Artificial Sequence
  RNAI TARGET SEQUENCE
gaaggattgtggttatgga

SEQ ID NO: 212 - DNA, 19 bp, Artificial Sequence
  RNAI TARGET SEQUENCE
ggagtctggtttaacctca

SEQ ID NO: 213 - DNA, 19 bp, Artificial Sequence
  RNAI TARGET SEQUENCE
gctacatgaccgatggact

SEQ ID NO: 214 - DNA, 19 bp, Artificial Sequence
  RNAI TARGET SEQUENCE
gcaagtttgtgccctcaga

SEQ ID NO: 215 - DNA, 19 bp, Artificial Sequence
  RNAI TARGET SEQUENCE
ctgacaaagagcttccact

SEQ ID NO: 216 - DNA, 19 bp, Artificial Sequence
  RNAI TARGET SEQUENCE
cccataacggagaatctgt

SEQ ID NO: 217 - DNA, 19 bp, Artificial Sequence
  RNAI TARGET SEQUENCE
ccaatgatggccattcagt

SEQ ID NO: 218 - DNA, 19 bp, Artificial Sequence
  RNAI TARGET SEQUENCE
ccttcaacattgaccgaaa

SEQ ID NO: 219 - DNA, 19 bp, Artificial Sequence
  RNAI TARGET SEQUENCE
cattcgaaaccacaaatct

SEQ ID NO: 220 - DNA, 19 bp, Artificial Sequence
  RNAI TARGET SEQUENCE
gaaatactgtctcccttta

SEQ ID NO: 221 - DNA, 19 bp, Artificial Sequence
  RNAI TARGET SEQUENCE
gaaatactaccatcttact

SEQ ID NO: 222 - DNA, 19 bp, Artificial Sequence
  RNAI TARGET SEQUENCE
ggtgtaaattaagtagcaa

SEQ ID NO: 223 - DNA, 19 bp, Artificial Sequence
  RNAI TARGET SEQUENCE
cttactcttctcacatgga

SEQ ID NO: 224 - DNA, 19 bp, Artificial Sequence
  RNAI TARGET SEQUENCE
ctattggatttaccatgta

SEQ ID NO: 225 - DNA, 19 bp, Artificial Sequence
  RNAI TARGET SEQUENCE
gagatggtgtactgctaca

SEQ ID NO: 226 - DNA, 19 bp, Artificial Sequence
  RNAI TARGET SEQUENCE
gtacttccgagaactcttt

SEQ ID NO: 227 - DNA, 19 bp, Artificial Sequence
  RNAI TARGET SEQUENCE
gcagtatgtggccagttat

SEQ ID NO: 228 - DNA, 19 bp, Artificial Sequence
  RNAI TARGET SEQUENCE
cagcatttgtcaaacgtca

SEQ ID NO: 229 - DNA, 19 bp, Artificial Sequence
  RNAI TARGET SEQUENCE
gtatgaaacggctagaatt

SEQ ID NO: 230 - DNA, 19 bp, Artificial Sequence
  RNAI TARGET SEQUENCE
ctcagcagtggatgaccaa

SEQ ID NO: 231 - DNA, 19 bp, Artificial Sequence
  RNAI TARGET SEQUENCE
ccttcttcctctccatgta

SEQ ID NO: 232 - DNA, 19 bp, Artificial Sequence
  RNAI TARGET SEQUENCE
ctcttggactaccttatga

SEQ ID NO: 233 - DNA, 19 bp, Artificial Sequence
  RNAI TARGET SEQUENCE
ctttggacctcgactcatt

SEQ ID NO: 234 - DNA, 19 bp, Artificial Sequence
  RNAI TARGET SEQUENCE
ctcagaatttgctgaccct

SEQ ID NO: 235 - DNA, 19 bp, Artificial Sequence
  RNAI TARGET SEQUENCE
gatgtcaagtcatattaca

SEQ ID NO: 236 - DNA, 19 bp, Artificial Sequence
  RNAI TARGET SEQUENCE
gatagtttattgggtgctt

SEQ ID NO: 237 - DNA, 19 bp, Artificial Sequence
  RNAI TARGET SEQUENCE
caagaagaagcgtttctga

SEQ ID NO: 238 - DNA, 19 bp, Artificial Sequence
  RNAI TARGET SEQUENCE
gctttcaactgtatggtct

SEQ ID NO: 239 - DNA, 19 bp, Artificial Sequence
  RNAI TARGET SEQUENCE
gatactgggtgtttaagga

SEQ ID NO: 240 - DNA, 19 bp, Artificial Sequence
  RNAI TARGET SEQUENCE
ggaattgtacctcgatctt

SEQ ID NO: 241 - DNA, 19 bp, Artificial Sequence
  RNAI TARGET SEQUENCE
cttgtaagtgggtcaccaa

SEQ ID NO: 242 - DNA, 19 bp, Artificial Sequence
  RNAI TARGET SEQUENCE
gaaatatatagctttggaa

SEQ ID NO: 243 - DNA, 19 bp, Artificial Sequence
  RNAI TARGET SEQUENCE
cactgtacctctaattaca

SEQ ID NO: 244 - DNA, 19 bp, Artificial Sequence
  RNAI TARGET SEQUENCE
cctattggaatctgggcta

SEQ ID NO: 245 - DNA, 19 bp, Artificial Sequence
  RNAI TARGET SEQUENCE
tgtttatgaccctggctta

SEQ ID NO: 246 - DNA, 19 bp, Artificial Sequence
  RNAI TARGET SEQUENCE
ccaaagatggcattgacca

SEQ ID NO: 247 - DNA, 19 bp, Artificial Sequence
  RNAI TARGET SEQUENCE
gtttgcatccggcacctga

SEQ ID NO: 248 - DNA, 19 bp, Artificial Sequence
  RNAI TARGET SEQUENCE
gtctatgtcttcattcaga

SEQ ID NO: 249 - DNA, 19 bp, Artificial Sequence
  RNAI TARGET SEQUENCE
catggatttggatgtagta

SEQ ID NO: 250 - DNA, 19 bp, Artificial Sequence
  RNAI TARGET SEQUENCE
ccaattggacttaacatgt

SEQ ID NO: 251 - DNA, 19 bp, Artificial Sequence
  RNAI TARGET SEQUENCE
ccttcactatggtgcgcta

SEQ ID NO: 252 - DNA, 19 bp, Artificial Sequence
  RNAI TARGET SEQUENCE
gagaactcatggtttaact

SEQ ID NO: 253 - DNA, 19 bp, Artificial Sequence
  RNAI TARGET SEQUENCE
ggagatttctcacgatgaa

SEQ ID NO: 254 - DNA, 19 bp, Artificial Sequence
  RNAI TARGET SEQUENCE
catttgctatgaacacgca

SEQ ID NO: 255 - DNA, 19 bp, Artificial Sequence
  RNAI TARGET SEQUENCE
gccttattatgatcttgta

SEQ ID NO: 256 - DNA, 19 bp, Artificial Sequence
  RNAI TARGET SEQUENCE
ctgttcaagaaagaggtga

SEQ ID NO: 257 - DNA, 19 bp, Artificial Sequence
  RNAI TARGET SEQUENCE
gaattctacttcccaacat

SEQ ID NO: 258 - DNA, 19 bp, Artificial Sequence
  RNAI TARGET SEQUENCE
caggttacatatcagttga

SEQ ID NO: 259 - DNA, 19 bp, Artificial Sequence
  RNAI TARGET SEQUENCE
ccacctatccctctggaca

SEQ ID NO: 260 - DNA, 19 bp, Artificial Sequence
  RNAI TARGET SEQUENCE
ccaaggagcagcgactcaa

SEQ ID NO: 261 - DNA, 19 bp, Artificial Sequence
  RNAI TARGET SEQUENCE
cacctttgagcaatggtct

SEQ ID NO: 262 - DNA, 19 bp, Artificial Sequence
  RNAI TARGET SEQUENCE
gccaatatagatctgctta

SEQ ID NO: 263 - DNA, 19 bp, Artificial Sequence
  RNAI TARGET SEQUENCE
gagattggactctgcagta

SEQ ID NO: 264 - DNA, 19 bp, Artificial Sequence
  RNAI TARGET SEQUENCE
ggaacttaaggaaaccttt

SEQ ID NO: 265 - DNA, 19 bp, Artificial Sequence
  RNAI TARGET SEQUENCE
gtataatgctaaagacaca

SEQ ID NO: 266 - DNA, 19 bp, Artificial Sequence
  RNAI TARGET SEQUENCE
gtgcatttcgggtagggat

SEQ ID NO: 267 - DNA, 19 bp, Artificial Sequence
  RNAI TARGET SEQUENCE
cattgacagatattgggca

SEQ ID NO: 268 - DNA, 19 bp, Artificial Sequence
  RNAI TARGET SEQUENCE
ctcaaaggaggtcatgttt

SEQ ID NO: 269 - DNA, 19 bp, Artificial Sequence
  RNAI TARGET SEQUENCE
ggatatttgccacagcatt

SEQ ID NO: 270 - DNA, 19 bp, Artificial Sequence
  RNAI TARGET SEQUENCE
cattgagtactctggcatt

SEQ ID NO: 271 - DNA, 19 bp, Artificial Sequence
  RNAI TARGET SEQUENCE
gaattggtggcagaacaga

SEQ ID NO: 272 - DNA, 19 bp, Artificial Sequence
  RNAI TARGET SEQUENCE
gctcttaccagacaatcaa

SEQ ID NO: 273 - DNA, 19 bp, Artificial Sequence
  RNAI TARGET SEQUENCE
ctaatgaacaggtgttgaa

SEQ ID NO: 274 - DNA, 19 bp, Artificial Sequence
  RNAI TARGET SEQUENCE
ctacctcgtgggatcccat

SEQ ID NO: 275 - DNA, 19 bp, Artificial Sequence
  RNAI TARGET SEQUENCE
cgctctacttctgcttcgt

SEQ ID NO: 276 - DNA, 19 bp, Artificial Sequence
  RNAI TARGET SEQUENCE
ctaagaacatgatgtggta

SEQ ID NO: 277 - DNA, 19 bp, Artificial Sequence
  RNAI TARGET SEQUENCE
cagtcaaagacatatgaga

SEQ ID NO: 278 - DNA, 19 bp, Artificial Sequence
  RNAI TARGET SEQUENCE
ctcaatggaggacttgaat

SEQ ID NO: 279 - DNA, 19 bp, Artificial Sequence
  RNAI TARGET SEQUENCE
gattataccttaactatgt

SEQ ID NO: 280 - DNA, 19 bp, Artificial Sequence
  RNAI TARGET SEQUENCE
ccatgctagtggtcctcat

SEQ ID NO: 281 - DNA, 19 bp, Artificial Sequence
  RNAI TARGET SEQUENCE
ctcatcaaatcccgaggga

SEQ ID NO: 282 - DNA, 19 bp, Artificial Sequence
  RNAI TARGET SEQUENCE
gacactatagtcgagccaa

SEQ ID NO: 283 - DNA, 19 bp, Artificial Sequence
  RNAI TARGET SEQUENCE
ccttaagtccagcaacata

SEQ ID NO: 284 - DNA, 19 bp, Artificial Sequence
  RNAI TARGET SEQUENCE
gaggaaattgccaggatca

SEQ ID NO: 285 - DNA, 19 bp, Artificial Sequence
  RNAI TARGET SEQUENCE
gtctcaaggtactttacga

SEQ ID NO: 286 - DNA, 19 bp, Artificial Sequence
  RNAI TARGET SEQUENCE
ggttatacaggttgttaaa

SEQ ID NO: 287 - DNA, 19 bp, Artificial Sequence
  RNAI TARGET SEQUENCE
cctattggatctcccagct

SEQ ID NO: 288 - DNA, 19 bp, Artificial Sequence
  RNAI TARGET SEQUENCE
gatcttcggaacttagtga

SEQ ID NO: 289 - DNA, 19 bp, Artificial Sequence
  RNAI TARGET SEQUENCE
ccattaaccagagaatagt

SEQ ID NO: 290 - DNA, 19 bp, Artificial Sequence
  RNAI TARGET SEQUENCE
ctcgaaagcaaagagataa

SEQ ID NO: 291 - DNA, 19 bp, Artificial Sequence
  RNAI TARGET SEQUENCE
ggaacattctagaatctgt

SEQ ID NO: 292 - DNA, 19 bp, Artificial Sequence
  RNAI TARGET SEQUENCE
cacattgagagagtgtata

SEQ ID NO: 293 - DNA, 19 bp, Artificial Sequence
  RNAI TARGET SEQUENCE
ccatcatttgtattccgga

SEQ ID NO: 294 - DNA, 19 bp, Artificial Sequence
  RNAI TARGET SEQUENCE
cagttcaaggctgcgctca

SEQ ID NO: 295 - DNA, 19 bp, Artificial Sequence
  RNAI TARGET SEQUENCE
ggattacaacctgagagtt

SEQ ID NO: 296 - DNA, 19 bp, Artificial Sequence
  RNAI TARGET SEQUENCE
cttgttgcctcacatccga

SEQ ID NO: 297 - DNA, 19 bp, Artificial Sequence
  RNAI TARGET SEQUENCE
gaaattattgggcgtcata

SEQ ID NO: 298 - DNA, 19 bp, Artificial Sequence
  RNAI TARGET SEQUENCE
ccatcaagcagggtttcaa

SEQ ID NO: 299 - DNA, 19 bp, Artificial Sequence
  RNAI TARGET SEQUENCE
ggaaatgatgactttatgt

SEQ ID NO: 300 - DNA, 19 bp, Artificial Sequence
  RNAI TARGET SEQUENCE
gacaattcagagagtcaca

SEQ ID NO: 301 - DNA, 19 bp, Artificial Sequence
  RNAI TARGET SEQUENCE
ccaataatgtcacctcgaa

SEQ ID NO: 302 - DNA, 19 bp, Artificial Sequence
  RNAI TARGET SEQUENCE
gacctagatgtcattgttt

SEQ ID NO: 303 - DNA, 19 bp, Artificial Sequence
  RNAI TARGET SEQUENCE
gaagtttgaccgctacaaa

SEQ ID NO: 304 - DNA, 19 bp, Artificial Sequence
  RNAI TARGET SEQUENCE
gtgtatgccgcaacggcta

SEQ ID NO: 305 - DNA, 19 bp, Artificial Sequence
  RNAI TARGET SEQUENCE
cagctaatcatcaacccaa

SEQ ID NO: 306 - DNA, 19 bp, Artificial Sequence
  RNAI TARGET SEQUENCE
caatttgaaccaagagtct

SEQ ID NO: 307 - DNA, 19 bp, Artificial Sequence
  RNAI TARGET SEQUENCE
cagataccgggcccaacga

SEQ ID NO: 308 - DNA, 19 bp, Artificial Sequence
  RANI TARGET SEQUENCE
cagttctcgatcaccggta

SEQ ID NO: 309 - DNA, 19 bp, Artificial Sequence
  RNAI TARGET SEQUENCE
ggatcaaccttcctatcca

SEQ ID NO: 310 - DNA, 19 bp, Artificial Sequence
  RNAI TARGET SEQUENCE
catatgatctccaccatga

SEQ ID NO: 311 - DNA, 19 bp, Artificial Sequence
  RNAI TARGET SEQUENCE
gtatgtgcgctatttccga

SEQ ID NO: 312 - DNA, 19 bp, Artificial Sequence
  RNAI TARGET SEQUENCE
caatacagttccgcttgta

SEQ ID NO: 313 - DNA, 19 bp, Artificial Sequence
  RNAI TARGET SEQUENCE
gtgaatctcaactaccgaa

SEQ ID NO: 314 - DNA, 19 bp, Artificial Sequence
  RNAI TARGET SEQUENCE
ggaatacctttcatcgatt

SEQ ID NO: 315 - DNA, 19 bp, Artificial Sequence
  RNAI TARGET SEQUENCE
gacctatgatgcaccacat

SEQ ID NO: 316 - DNA, 19 bp, Artificial Sequence
  RNAI TARGET SEQUENCE
ggcaacagttcttcatcga

SEQ ID NO: 317 - DNA, 19 bp, Artificial Sequence
  RNAI TARGET SEQUENCE
ccggaaatacagattgatt

SEQ ID NO: 318 - DNA, 19 bp, Artificial Sequence
  RNAI TARGET SEQUENCE
cgcgaggtgcacttcatga

SEQ ID NO: 319 - DNA, 19 bp, Artificial Sequence
  RNAI TARGET SEQUENCE
ccatgaacgccgaggacga

SEQ ID NO: 320 - DNA, 19 bp, Artificial Sequence
  RNAI TARGET SEQUENCE
cattctaaaggtcagtgta

SEQ ID NO: 321 - DNA, 19 bp, Artificial Sequence
  RNAI TARGET SEQUENCE
gatcatctgccttccgaaa

SEQ ID NO: 322 - DNA, 19 bp, Artificial Sequence
  RNAI TARGET SEQUENCE
cctactttggcctgcttga

SEQ ID NO: 323 - DNA, 19 bp, Artificial Sequence
  RNAI TARGET SEQUENCE
cttactacaacaatctgta

SEQ ID NO: 324 - DNA, 19 bp, Artificial Sequence
  RNAI TARGET SEQUENCE
ggaatatcttctaatggga

SEQ ID NO: 325 - DNA, 19 bp, Artificial Sequence
  RNAI TARGET SEQUENCE
cgtgttagccttacaccaa

SEQ ID NO: 326 - DNA, 19 bp, Artificial Sequence
  RNAI TARGET SEQUENCE
gtaattgagtataagtcca

SEQ ID NO: 327 - DNA, 19 bp, Artificial Sequence
  RNAI TARGET SEQUENCE
gtttgacattgagattgga

SEQ ID NO: 328 - DNA, 19 bp, Artificial Sequence
  RNAI TARGET SEQUENCE
cagacttctttgagtactt

SEQ ID NO: 329 - DNA, 19 bp, Artificial Sequence
  RNAI TARGET SEQUENCE
cagtgaatcaggtggcgaa

SEQ ID NO: 330 - DNA, 19 bp, Artificial Sequence
  RNAI TARGET SEQUENCE
gggataagcctcctttctt

SEQ ID NO: 331 - DNA, 19 bp, Artificial Sequence
  RNAI TARGET SEQUENCE
cacaatgtggtccacatga

SEQ ID NO: 332 - DNA, 19 bp, Artificial Sequence
  RNAI TARGET SEQUENCE
gtacctctcctatagacat

SEQ ID NO: 333 - DNA, 19 bp, Artificial Sequence
  RNAI TARGET SEQUENCE
cggtattgatgatgtgcct

SEQ ID NO: 334 - DNA, 19 bp, Artificial Sequence
  RNAI TARGET SEQUENCE
cttgaacttccacgaggta

SEQ ID NO: 335 - DNA, 19 bp, Artificial Sequence
  RNAI TARGET SEQUENCE
cataagattggctggaatt

SEQ ID NO: 336 - DNA, 19 bp, Artificial Sequence
  RNAI TARGET SEQUENCE
gcacctacctgggtcactt

SEQ ID NO: 337 - DNA, 19 bp, Artificial Sequence
  RNAI TARGET SEQUENCE
cttagactggaaattaaga

SEQ ID NO: 338 - DNA, 19 bp, Artificial Sequence
  RNAI TARGET SEQUENCE
gagctaaagctgtaaccca

SEQ ID NO: 339 - DNA, 19 bp, Artificial Sequence
  RNAI TARGET SEQUENCE
ggtgataggtaagaatatt

SEQ ID NO: 340 - DNA, 19 bp, Artificial Sequence
  RNAI TARGET SEQUENCE
gagatgatggactcatgta

SEQ ID NO: 341 - DNA, 19 bp, Artificial Sequence
  RNAI TARGET SEQUENCE
caccttatatcccaaatgt

SEQ ID NO: 342 - DNA, 19 bp, Artificial Sequence
  RNAI TARGET SEQUENCE
cctacgtcttcaacatcat

SEQ ID NO: 343 - DNA, 19 bp, Artificial Sequence
  RNAI TARGET SEQUENCE
catcaagcagggtttcaaa

SEQ ID NO: 344 - DNA, 21 bp, Artificial Sequence
  siRNA ANTISENSE SEQUENCE
uuacaaaccgcucuaguuctt

SEQ ID NO: 345 - DNA, 21 bp, Artificial Sequence
  siRNA SENSE SEQUENCE
gaacuagagcgguuuguaatt

SEQ ID NO: 346 - DNA, 21 bp, Artificial Sequence
  siRNA ANTISENSE SEQUENCE
aucugauuguuaaaggaagtt

SEQ ID NO: 347 - DNA, 21 bp, Artificial Sequence
  siRNA SENSE SEQUENCE
cuuccuuuaacaaucagautt

SEQ ID NO: 348 - DNA, 21 bp, Artificial Sequence
  siRNA ANTISENSE SEQUENCE
uuuguccuggauauaaguctt

SEQ ID NO: 349 - DNA, 21 bp, Artificial Sequence
  siRNA SENSE SEQUENCE
gacuuauauccaggacaaatt

SEQ ID NO: 350 - DNA, 21 bp, Artificial Sequence
  siRNA ANTISENSE SEQUENCE
aacaacacccuguugaucctt

SEQ ID NO: 351 - DNA, 21 bp, Artificial Sequence
  siRNA SENSE SEQUENCE
ggaucaacaggguguuguutt

SEQ ID NO: 352 - DNA, 21 bp, Artificial Sequence
  siRNA ANTISENSE SEQUENCE
uauacuuccuggauaaacctt

SEQ ID NO: 353 - DNA, 21 bp, Artificial Sequence
  siRNA SENSE SEQUENCE
gguuuauccaggaaguauatt

SEQ ID NO: 354 - DNA, 21 bp, Artificial Sequence
  siRNA ANTISENSE SEQUENCE
uuacucuccaggauuccugtt

SEQ ID NO: 355 - DNA, 21 bp, Artificial Sequence
  siRNA SENSE SEQUENCE
caggaauccuggagaguaatt

SEQ ID NO: 356 - DNA, 21 bp, Artificial Sequence
  siRNA ANTISENSE SEQUENCE
uagcccaagaugauacucctt

SEQ ID NO: 357 - DNA, 21 bp, Artificial Sequence
  siRNA SENSE SEQUENCE
ggaguaucaucuugggcuatt

SEQ ID NO: 358 - DNA, 21 bp, Artificial Sequence
  siRNA ANTISENSE SEQUENCE
aggcagaucacacacucactt

SEQ ID NO: 359 - DNA, 21 bp, Artificial Sequence
  siRNA SENSE SEQUENCE
gugagugugugaucugccutt

SEQ ID NO: 360 - DNA, 21 bp, Artificial Sequence
  siRNA ANTISENSE SEQUENCE
aagaacaccuucaucaucctt

SEQ ID NO: 361 - DNA, 21 bp, Artificial Sequence
  siRNA SENSE SEQUENCE
ggaugaugaagguguucuutt

SEQ ID NO: 362 - DNA, 21 bp, Artificial Sequence
  siRNA ANTISENSE SEQUENCE
uuguuggcuauccaaaucgtt

SEQ ID NO: 363 - DNA, 21 bp, Artificial Sequence
  siRNA SENSE SEQUENCE
cgauuuggauagccaacaatt

SEQ ID NO: 364 - DNA, 21 bp, Artificial Sequence
  siRNA ANTISENSE SEQUENCE
aaguacaagccauauuuggtt

SEQ ID NO: 365 - DNA, 21 bp, Artificial Sequence
  siRNA SENSE SEQUENCE
ccaaauauggcuuguacuutt

SEQ ID NO: 366 - DNA, 21 bp, Artificial Sequence
  siRNA ANTISENSE SEQUENCE
uaggucaugaaguaaguggtt

SEQ ID NO: 367 - DNA, 21 bp, Artificial Sequence
  siRNA SENSE SEQUENCE
ccacuuacuucaugaccuatt

SEQ ID NO: 368 - DNA, 21 bp, Artificial Sequence
  siRNA ANTISENSE SEQUENCE
auugaacagccaugcaauctt

SEQ ID NO: 369 - DNA, 21 bp, Artificial Sequence
  siRNA SENSE SEQUENCE
gauugcauggcuguucaautt

SEQ ID NO: 370 - DNA, 21 bp, Artificial Sequence
  siRNA ANTISENSE SEQUENCE
ucugucccgaugcuagcugtt

SEQ ID NO: 371 - DNA, 21 bp, Artificial Sequence
  siRNA SENSE SEQUENCE
cagcuagcaucgggacagatt

SEQ ID NO: 372 - DNA, 21 bp, Artificial Sequence
  siRNA ANTISENSE SEQUENCE
uuacucuguggcucaauuctt

SEQ ID NO: 373 - DNA, 21 bp, Artificial Sequence
  siRNA SENSE SEQUENCE
gaauugagccacagaguaatt

SEQ ID NO: 374 - DNA, 21 bp, Artificial Sequence
  siRNA ANTISENSE SEQUENCE
uaagaaagccuucaguuuctt

SEQ ID NO: 375 - DNA, 21 bp, Artificial Sequence
  siRNA SENSE SEQUENCE
gaaacugaaggcuuucuuatt

SEQ ID NO: 376 - DNA, 21 bp, Artificial Sequence
  siRNA ANTISENSE SEQUENCE
aaacuuagauguguaguuctt

SEQ ID NO: 377 - DNA, 21 bp, Artificial Sequence
  siRNA SENSE SEQUENCE
gaacuacacaucuaaguuutt

SEQ ID NO: 378 - DNA, 21 bp, Artificial Sequence
  siRNA ANTISENSE SEQUENCE
uggucgaagaugaacugugtt

SEQ ID NO: 379 - DNA, 21 bp, Artificial Sequence
  siRNA SENSE SEQUENCE
cacaguucaucuucgaccatt

SEQ ID NO: 380 - DNA, 21 bp, Artificial Sequence
  siRNA ANTISENSE SEQUENCE
aaaucugcaaacuucuuugtt

SEQ ID NO: 381 - DNA, 21 bp, Artificial Sequence
  siRNA SENSE SEQUENCE
caaagaaguuugcagauuutt

SEQ ID NO: 382 - DNA, 21 bp, Artificial Sequence
  siRNA ANTISENSE SEQUENCE
uacggucaaauccuucuugtt

SEQ ID NO: 383 - DNA, 21 bp, Artificial Sequence
  siRNA SENSE SEQUENCE
caagaaggauuugaccguatt

SEQ ID NO: 384 - DNA, 21 bp, Artificial Sequence
  siRNA ANTISENSE SEQUENCE
uuggcgaccaucuggauagtt

SEQ ID NO: 385 - DNA, 21 bp, Artificial Sequence
  siRNA SENSE SEQUENCE
cuauccagauggucgccaatt

SEQ ID NO: 386 - DNA, 21 bp, Artificial Sequence
  siRNA ANTISENSE SEQUENCE
uccacucaaugauaaagggtt

SEQ ID NO: 387 - DNA, 21 bp, Artificial Sequence
  siRNA SENSE SEQUENCE
cccuuuaucauugaguggatt

SEQ ID NO: 388 - DNA, 21 bp, Artificial Sequence
  siRNA ANTISENSE SEQUENCE
ucauggauggaaauagcagtt

SEQ ID NO: 389 - DNA, 21 bp, Artificial Sequence
  siRNA SENSE SEQUENCE
cugcuauuuccauccaugatt

SEQ ID NO: 390 - DNA, 21 bp, Artificial Sequence
  siRNA ANTISENSE SEQUENCE
uggcaauugaagguauaggtt

SEQ ID NO: 391 - DNA, 21 bp, Artificial Sequence
  siRNA SENSE SEQUENCE
ccuauaccuucaauugccatt

SEQ ID NO: 392 - DNA, 21 bp, Artificial Sequence
  siRNA ANTISENSE SEQUENCE
uaugauuggcacucuagagtt

SEQ ID NO: 393 - DNA, 21 bp, Artificial Sequence
  siRNA SENSE SEQUENCE
cucuagagugccaaucauatt

SEQ ID NO: 394 - DNA, 21 bp, Artificial Sequence
  siRNA ANTISENSE SEQUENCE
auguguaucgguagaucuctt

SEQ ID NO: 395 - DNA, 21 bp, Artificial Sequence
  siRNA SENSE SEQUENCE
gagaucuaccgauacacautt

SEQ ID NO: 396 - DNA, 21 bp, Artificial Sequence
  siRNA ANTISENSE SEQUENCE
uccaugauacaagaaucggtt

SEQ ID NO: 397 - DNA, 21 bp, Artificial Sequence
  siRNA SENSE SEQUENCE
ccgauucuuguaucauggatt

SEQ ID NO: 398 - DNA, 21 bp, Artificial Sequence
  siRNA ANTISENSE SEQUENCE
ucgaucaccagcucauaagtt

SEQ ID NO: 399 - DNA, 21 bp, Artificial Sequence
  siRNA SENSE SEQUENCE
cuuaugagcuggugaucgatt

SEQ ID NO: 400 - DNA, 21 bp, Artificial Sequence
  siRNA ANTISENSE SEQUENCE
uucaagaucacacacauagtt

SEQ ID NO: 401 - DNA, 21 bp, Artificial Sequence
  siRNA SENSE SEQUENCE
cuaugugugugaucuugaatt

SEQ ID NO: 402 - DNA, 21 bp, Artificial Sequence
  siRNA ANTISENSE SEQUENCE
ugagguuaaaccagacucctt

SEQ ID NO: 403 - DNA, 21 bp, Artificial Sequence
  siRNA SENSE SEQUENCE
ggagucugguuuaaccucatt

SEQ ID NO: 404 - DNA, 21 bp, Artificial Sequence
  siRNA ANTISENSE SEQUENCE
uauccucgaaguuugguagtt

SEQ ID NO: 405 - DNA, 21 bp, Artificial Sequence
  siRNA SENSE SEQUENCE
cuaccaaacuucgaggauatt

SEQ ID NO: 406 - DNA, 21 bp, Artificial Sequence
  siRNA ANTISENSE SEQUENCE
uacacgaagguccucaaugtt

SEQ ID NO: 407 - DNA, 21 bp, Artificial Sequence
  siRNA SENSE SEQUENCE
cauugaggaccuucguguatt

SEQ ID NO: 408 - DNA, 21 bp, Artificial Sequence
  siRNA ANTISENSE SEQUENCE
acugaauggccaucauuggtt

SEQ ID NO: 409 - DNA, 21 bp, Artificial Sequence
  siRNA SENSE SEQUENCE
ccaaugauggccauucagutt

SEQ ID NO: 410 - DNA, 21 bp, Artificial Sequence
  siRNA ANTISENSE SEQUENCE
uugaggagccgguuguuagtt

SEQ ID NO: 411 - DNA, 21 bp, Artificial Sequence
  siRNA SENSE SEQUENCE
cuaacaaccggcuccucaatt

SEQ ID NO: 412 - DNA, 21 bp, Artificial Sequence
  siRNA ANTISENSE SEQUENCE
uuucggucaauguugaaggtt

SEQ ID NO: 413 - DNA, 21 bp, Artificial Sequence
  siRNA SENSE SEQUENCE
ccuucaacauugaccgaaatt

SEQ ID NO: 414 - DNA, 21 bp, Artificial Sequence
  siRNA ANTISENSE SEQUENCE
aacaucacugaauaauggctt

SEQ ID NO: 415 - DNA, 21 bp, Artificial Sequence
  siRNA SENSE SEQUENCE
gccauuauucagugauguutt

SEQ ID NO: 416 - DNA, 21 bp, Artificial Sequence
  siRNA ANTISENSE SEQUENCE
ucugagggcacaaacuugctt

SEQ ID NO: 417 - DNA, 21 bp, Artificial Sequence
  siRNA SENSE SEQUENCE
gcaaguuugugcccucagatt

SEQ ID NO: 418 - DNA, 21 bp, Artificial Sequence
  siRNA ANTISENSE SEQUENCE
aguggaagcucuuugucagtt

SEQ ID NO: 419 - DNA, 21 bp, Artificial Sequence
  siRNA SENSE SEQUENCE
cugacaaagagcuuccacutt

SEQ ID NO: 420 - DNA, 21 bp, Artificial Sequence
  siRNA ANTISENSE SEQUENCE
uguaauuagagguacagugtt

SEQ ID NO: 421 - DNA, 21 bp, Artificial Sequence
  siRNA SENSE SEQUENCE
cacuguaccucuaauuacatt

SEQ ID NO: 422 - DNA, 21 bp, Artificial Sequence
  siRNA ANTISENSE SEQUENCE
agaccauacaguugaaagctt

SEQ ID NO: 423 - DNA, 21 bp, Artificial Sequence
  siRNA SENSE SEQUENCE
gcuuucaacuguauggucutt

SEQ ID NO: 424 - DNA, 21 bp, Artificial Sequence
  siRNA ANTISENSE SEQUENCE
uuggucauccacugcugagtt

SEQ ID NO: 425 - DNA, 21 bp, Artificial Sequence
  siRNA SENSE SEQUENCE
cucagcaguggaugaccaatt

SEQ ID NO: 426 - DNA, 21 bp, Artificial Sequence
  siRNA ANTISENSE SEQUENCE
auaacuggccacauacugctt

SEQ ID NO: 427 - DNA, 21 bp, Artificial Sequence
  siRNA SENSE SEQUENCE
gcaguauguggccaguuautt

SEQ ID NO: 428 - DNA, 21 bp, Artificial Sequence
  siRNA ANTISENSE SEQUENCE
uacauggagaggaagaaggtt

SEQ ID NO: 429 - DNA, 21 bp, Artificial Sequence
  siRNA SENSE SEQUENCE
ccuucuuccucuccauguatt

SEQ ID NO: 430 - DNA, 21 bp, Artificial Sequence
  siRNA ANTISENSE SEQUENCE
uguaauaugacuugacauctt

SEQ ID NO: 431 - DNA, 21 bp, Artificial Sequence
  siRNA SENSE SEQUENCE
gaugucaagucauauuacatt

SEQ ID NO: 432 - DNA, 21 bp, Artificial Sequence
  siRNA ANTISENSE SEQUENCE
uccaugugagaagaguaagtt

SEQ ID NO: 433 - DNA, 21 bp, Artificial Sequence
  siRNA SENSE SEQUENCE
cuuacucuucucacauggatt

SEQ ID NO: 434 - DNA, 21 bp, Artificial Sequence
  siRNA ANTISENSE SEQUENCE
ugacguuugacaaaugcugtt

SEQ ID NO: 435 - DNA, 21 bp, Artificial Sequence
  SiRNA SENSE SEQUENCE
cagcauuugucaaacgucatt

SEQ ID NO: 436 - DNA, 21 bp, Artificial Sequence
  SiRNA ANTISENSE SEQUENCE
uuggugacccacuuacaagtt

SEQ ID NO: 437 - DNA, 21 bp, Artificial Sequence
  SiRNA SENSE SEQUENCE
cuuguaagugggucaccaatt

SEQ ID NO: 438 - DNA, 21 bp, Artificial Sequence
  SiRNA ANTISENSE SEQUENCE
uucaucgugagaaaucucctt

SEQ ID NO: 439 - DNA, 21 bp, Artificial Sequence
  SiRNA SENSE SEQUENCE
ggagauuucucacgaugaatt

SEQ ID NO: 440 - DNA, 21 bp, Artificial Sequence
  SiRNA ANTISENSE SEQUENCE
aaugcuguggcaaauaucctt

SEQ ID NO: 441 - DNA, 21 bp, Artificial Sequence
  SiRNA SENSE SEQUENCE
ggauauuugccacagcauutt

SEQ ID NO: 442 - DNA, 21 bp, Artificial Sequence
  SiRNA ANTISENSE SEQUENCE
uggucaaugccaucuuuggtt

SEQ ID NO: 443 - DNA, 21 bp, Artificial Sequence
  SiRNA SENSE SEQUENCE
ccaaagauggcauugaccatt

SEQ ID NO: 444 - DNA, 21 bp, Artificial Sequence
  siRNA ANTISENSE SEQUENCE
aucccuacccgaaaugcactt

SEQ ID NO: 445 - DNA, 21 bp, Artificial Sequence
  siRNA SENSE SEQUENCE
gugcauuucggguagggautt

SEQ ID NO: 446 - DNA, 21 bp, Artificial Sequence
  siRNA ANTISENSE SEQUENCE
uaagcagaucuauauuggctt

SEQ ID NO: 447 - DNA, 21 bp, Artificial Sequence
  siRNA SENSE SEQUENCE
gccaauauagaucugcuuatt

SEQ ID NO: 448 - DNA, 21 bp, Artificial Sequence
  siRNA ANTISENSE SEQUENCE
ucucauaugucuuugacugtt

SEQ ID NO: 449 - DNA, 21 bp, Artificial Sequence
  siRNA SENSE SEQUENCE
cagucaaagacauaugagatt

SEQ ID NO: 450 - DNA, 21 bp, Artificial Sequence
  siRNA ANTISENSE SEQUENCE
augaggaccacuagcauggtt

SEQ ID NO: 451 - DNA, 21 bp, Artificial Sequence
  siRNA SENSE SEQUENCE
ccaugcuagugguccucautt

SEQ ID NO: 452 - DNA, 21 bp, Artificial Sequence
  siRNA ANTISENSE SEQUENCE
uugauugucugguaagagctt

SEQ ID NO: 453 - DNA, 21 bp, Artificial Sequence
  siRNA SENSE SEQUENCE
gcucuuaccagacaaucaatt

SEQ ID NO: 454 - DNA, 21 bp, Artificial Sequence
  siRNA ANTISENSE SEQUENCE
acagauucuagaauguucctt

SEQ ID NO: 455 - DNA, 21 bp, Artificial Sequence
  siRNA SENSE SEQUENCE
ggaacauucuagaaucugutt

SEQ ID NO: 456 - DNA, 21 bp, Artificial Sequence
  siRNA ANTISENSE SEQUENCE
uuuaacaaccuguauaacctt

SEQ ID NO: 457 - DNA, 21 bp, Artificial Sequence
  siRNA SENSE SEQUENCE
gguuauacagguuguuaaatt

SEQ ID NO: 458 - DNA, 21 bp, Artificial Sequence
  siRNA ANTISENSE SEQUENCE
uccggaauacaaaugauggtt

SEQ ID NO: 459 - DNA, 21 bp, Artificial Sequence
  siRNA SENSE SEQUENCE
ccaucauuuguauuccggatt

SEQ ID NO: 460 - DNA, 21 bp, Artificial Sequence
  siRNA ANTISENSE SEQUENCE
uuaucucuuugcuuucgagtt

SEQ ID NO: 461 - DNA, 21 bp, Artificial Sequence
  siRNA SENSE SEQUENCE
cucgaaagcaaagagauaatt

SEQ ID NO: 462 - DNA, 21 bp, Artificial Sequence
  siRNA ANTISENSE SEQUENCE
uaccacaucauguucuuagtt

SEQ ID NO: 463 - DNA, 21 bp, Artificial Sequence
  siRNA SENSE SEQUENCE
cuaagaacaugaugugguatt

SEQ ID NO: 464 - DNA, 21 bp, Artificial Sequence
  siRNA ANTISENSE SEQUENCE
agcugggagauccaauaggtt

SEQ ID NO: 465 - DNA, 21 bp, Artificial Sequence
  siRNA SENSE SEQUENCE
ccuauuggaucucccagcutt

SEQ ID NO: 466 - DNA, 21 bp, Artificial Sequence
  siRNA ANTISENSE SEQUENCE
auguggugcaucauagguctt

SEQ ID NO: 467 - DNA, 21 bp, Artificial Sequence
  siRNA SENSE SEQUENCE
gaccuaugaugcaccacautt

SEQ ID NO: 468 - DNA, 21 bp, Artificial Sequence
  siRNA ANTISENSE SEQUENCE
ucaugguggagaucauaugtt

SEQ ID NO: 469 - DNA, 21 bp, Artificial Sequence
  siRNA SENSE SEQUENCE
cauaugaucuccaccaugatt

SEQ ID NO: 470 - DNA, 21 bp, Artificial Sequence
  siRNA ANTISENSE SEQUENCE
uuucggaaggcagaugauctt

SEQ ID NO: 471 - DNA, 21 bp, Artificial Sequence
  siRNA SENSE SEQUENCE
gaucaucugccuuccgaaatt

SEQ ID NO: 472 - DNA, 21 bp, Artificial Sequence
  siRNA ANTISENSE SEQUENCE
ucaagcaggccaaaguaggtt

SEQ ID NO: 473 - DNA, 21 bp, Artificial Sequence
  siRNA SENSE SEQUENCE
ccuacuuuggccugcuugatt

SEQ ID NO: 474 - DNA, 21 bp, Artificial Sequence
  siRNA ANTISENSE SEQUENCE
ucggaaauagcgcacauactt

SEQ ID NO: 475 - DNA, 21 bp, Artificial Sequence
  siRNA SENSE SEQUENCE
guaugugcgcuauuuccgatt

SEQ ID NO: 476 - DNA, 21 bp, Artificial Sequence
  siRNA ANTISENSE SEQUENCE
uagccguugcggcauacactt

SEQ ID NO: 477 - DNA, 21 bp, Artificial Sequence
  siRNA SENSE SEQUENCE
guguaugccgcaacggcuatt

SEQ ID NO: 478 - DNA, 21 bp, Artificial Sequence
  siRNA ANTISENSE SEQUENCE
uacaagcggaacuguauugtt

SEQ ID NO: 479 - DNA, 21 bp, Artificial Sequence
  siRNA SENSE SEQUENCE
caauacaguuccgcuuguatt

SEQ ID NO: 480 - DNA, 21 bp, Artificial Sequence
  siRNA ANTISENSE SEQUENCE
uuggguugaugauuagcugtt

SEQ ID NO: 481 - DNA, 21 bp, Artificial Sequence
  siRNA SENSE SEQUENCE
cagcuaaucaucaacccaatt

SEQ ID NO: 482 - DNA, 21 bp, Artificial Sequence
  siRNA ANTISENSE SEQUENCE
uucgguaguugagauucactt

SEQ ID NO: 483 - DNA, 21 bp, Artificial Sequence
  siRNA SENSE SEQUENCE
gugaaucucaacuaccgaatt

SEQ ID NO: 484 - DNA, 21 bp, Artificial Sequence
  siRNA ANTISENSE SEQUENCE
uacagauuguuguaguaagtt

SEQ ID NO: 485 - DNA, 21 bp, Artificial Sequence
  siRNA SENSE SEQUENCE
cuuacuacaacaaucuguatt

SEQ ID NO: 486 - DNA, 21 bp, Artificial Sequence
  siRNA ANTISENSE SEQUENCE
aggcacaucaucaauaccgtt

SEQ ID NO: 487 - DNA, 21 bp, Artificial Sequence
  siRNA SENSE SEQUENCE
cgguauugaugaugugccutt

SEQ ID NO: 488 - DNA, 21 bp, Artificial Sequence
  siRNA ANTISENSE SEQUENCE
augauguugaagacguaggtt

SEQ ID NO: 489 - DNA, 21 bp, Artificial Sequence
  siRNA SENSE SEQUENCE
ccuacgucuucaacaucautt

SEQ ID NO: 490 - DNA, 21 bp, Artificial Sequence
  siRNA ANTISENSE SEQUENCE
aauauucuuaccuaucacctt

SEQ ID NO: 491 - DNA, 21 bp, Artificial Sequence
  siRNA SENSE SEQUENCE
ggugauagguaagaauauutt

SEQ ID NO: 492 - DNA, 21 bp, Artificial Sequence
  siRNA ANTISENSE SEQUENCE
uccaaucucaaugucaaactt

SEQ ID NO: 493 - DNA, 21 bp, Artificial Sequence
  siRNA SENSE SEQUENCE
guuugacauugagauuggatt

SEQ ID NO: 494 - DNA, 5509 bp, Artificial Sequence
  EXPRESSION VECTOR COMBINING ELEMENTS FROM MULTIPLE ORGANISMS
gatctggatccgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 495 - DNA, 5237 bp, Artificial Sequence
  Expression vector combining elements from multiple organisms
gatctggatccttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatccgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 496 - DNA, 5659 bp, Artificial Sequence
  Expression vector combining elements from multiple organisms
gatctggatccttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatccgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggccaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaatttcaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 497 - DNA, 478 bp, Homo sapiens
cccgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgcctccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 498 - DNA, 66 bp, Artificial Sequence
  Wnk4 shRNA
gatacctcaagtttgacattgagattggagtctagagtccaatctcaatgtcaaacttgaggtatc

SEQ ID NO: 499 - DNA, 549 bp, Artificial Sequence
  Wnk4 shRNA EXPRESSION CASSETTE
cccgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgcctccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatacctcaagtttgacattgagattggagtctagagtccaatctcaatgtcaaacttgaggtatcttttt